SEQUENCE LISTING (304 sequences)

SEQ ID NO: 1 - protein, 8 aa, Artificial Sequence
  2A peptide consensus sequence
  MISC_FEATURE (2)..(2): Xaa is V or I
  MISC_FEATURE (4)..(4): Xaa is any amino acid
DXEXNPGP

SEQ ID NO: 2 - DNA, 11654 bp, Artificial Sequence
  RRV vector containing 2A-cassette
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagtgaaacagactttgaattttgaccttctcaagttggcgggagacgtggagtccaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 3 - DNA, 9 bp, Artificial Sequence
  GSG linker sequence
ggaagcgga

SEQ ID NO: 4 - DNA, 30 bp, Artificial Sequence
  GFP-R-Gib primer
taaaatcttttattttatctgcggccgcac

SEQ ID NO: 5 - protein, 11 aa, Artificial Sequence
  Peptide readthrough sequence
CAAADKIKDFI

SEQ ID NO: 6 - DNA, 33 bp, Artificial Sequence
  Ascl-yCD2 formard primer
gatcggcgcgcctatggtgaccggcggcatggc

SEQ ID NO: 7 - DNA, 24 bp, Artificial Sequence
  3-37 primer
cccctttttctggagactaaataa

SEQ ID NO: 8 - DNA, 54 bp, Artificial Sequence
  2A peptide sequence
gagggcagaggaagtcttctaacatgcggtgacgtggaggagaatcccggccct

SEQ ID NO: 9 - DNA, 63 bp, Artificial Sequence
  2A peptide sequence
ggaagcggagagggcagaggaagtcttctaacatgcggtgacgtggaggagaatcccggccct

SEQ ID NO: 10 - DNA, 57 bp, Artificial Sequence
  2A peptide sequence
gctactaacttcagcctgctgaagcaggctggagacgtggaggagaaccctggacct

SEQ ID NO: 11 - DNA, 66 bp, Artificial Sequence
  2A peptide sequence
ggaagcggagctactaacttcagcctgctgaagcaggctggagacgtggaggagaaccctggacct

SEQ ID NO: 12 - DNA, 66 bp, Artificial Sequence
  2A peptide sequence
gtgaaacagactttgaattttgaccttctcaagttggcgggagacgtggagtccaaccctggacct

SEQ ID NO: 13 - DNA, 75 bp, Artificial Sequence
  2A peptide sequence
ggaagcggagtgaaacagactttgaattttgaccttctcaagttggcgggagacgtggagtccaaccctggacct

SEQ ID NO: 14 - DNA, 60 bp, Artificial Sequence
  2A peptide sequence
cagtgtactaattatgctctcttgaaattggctggagatgttgagagcaaccctggacct

SEQ ID NO: 15 - DNA, 69 bp, Artificial Sequence
  2A peptide sequence
ggaagcggacagtgtactaattatgctctcttgaaattggctggagatgttgagagcaaccctggacct

SEQ ID NO: 16 - DNA, 54 bp, Artificial Sequence; identical to SEQ ID NO: 8
  2A peptide sequence

SEQ ID NO: 17 - DNA, 63 bp, Artificial Sequence; identical to SEQ ID NO: 9
  2A peptide sequence

SEQ ID NO: 18 - DNA, 57 bp, Artificial Sequence; identical to SEQ ID NO: 10
  2A peptide sequence

SEQ ID NO: 19 - DNA, 66 bp, Artificial Sequence; identical to SEQ ID NO: 11
  2A peptide sequence

SEQ ID NO: 20 - DNA, 19 bp, Artificial Sequence
  5-MLV-U3-R primer
agcccacaacccctcactc

SEQ ID NO: 21 - DNA, 18 bp, Artificial Sequence
  3-MLV-Psi primer sequence
tctcccgatcccggacga

SEQ ID NO: 22 - DNA, 26 bp, Artificial Sequence
  Probe sequence
ccccaaatgaaagacccccgctgacg

SEQ ID NO: 23 - DNA, 23 bp, Artificial Sequence
  IRES forward primer
ctgatcttactctttggaccttg

SEQ ID NO: 24 - DNA, 24 bp, Artificial Sequence
  IRES reverse primer
cccctttttctggagactaaataa

SEQ ID NO: 25 - DNA, 22 bp, Artificial Sequence
  ENV forward primer
accctcaacctcccctacaagt

SEQ ID NO: 26 - DNA, 20 bp, Artificial Sequence
  ENV reverse primer
gttaagcgcctgataggctc

SEQ ID NO: 27 - DNA, 26 bp, Artificial Sequence
  Env probe sequence
ccccaaatgaaagacccccgctgacg

SEQ ID NO: 28 - DNA, 477 bp, Artificial Sequence
  Human codon optimized heat stabilized CD coding sequence
atggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaa

SEQ ID NO: 29 - protein, 158 aa, Artificial Sequence
  Heat stabilized APOBEC modified CD polypeptide
  MISC_FEATURE (10)..(10): Xaa is any amino acid
  MISC_FEATURE (152)..(152): Xaa is any amino acid
MVTGGMASKXDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDXFEDIGE

SEQ ID NO: 30 - DNA, 54 bp, Artificial Sequence; identical to SEQ ID NO: 8
  2A peptide coding sequence

SEQ ID NO: 31 - DNA, 1062 bp, Artificial Sequence
  BstBI-env-T2A-GFPm of pAC3-T2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagagggcagaggaagtcttctaacatgcggtgacgtggaggagaatcccggccctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtag

SEQ ID NO: 32 - DNA, 1026 bp, Artificial Sequence
  BstBI-env-P2A-GFPm of pAC3-P2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagctactaacttcagcctgctgaagcaggctggagacgtggaggagaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 33 - DNA, 1029 bp, Artificial Sequence
  BstBI-env-E2A-GFPm of pAC3-E2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccacagtgtactaattatgctctcttgaaattggctggagatgttgagagcaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 34 - DNA, 1035 bp, Artificial Sequence
  BstBI-env-F2A-GFPm of pAC3-F2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagtgaaacagactttgaattttgaccttctcaagttggcgggagacgtggagtccaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 35 - DNA, 1032 bp, Artificial Sequence
  BstBI-env-GSG-T2A-GFPm of pAC3-GSG-T2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagagggcagaggaagtcttctaacatgcggtgacgtggaggagaatcccggccctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 36 - DNA, 1035 bp, Artificial Sequence
  BstBI-env-GSG-P2A-GFPm of pAC3-GSG-P2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagctactaacttcagcctgctgaagcaggctggagacgtggaggagaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 37 - DNA, 1044 bp, Artificial Sequence
  BstBI-env-GSG-F2A-GFPm of pAC3-GSG-F2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagtgaaacagactttgaattttgaccttctcaagttggcgggagacgtggagtccaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 38 - DNA, 1038 bp, Artificial Sequence
  BstBI-env-GSG-E2A-GFPm of pAC3-GSG-E2A-GFPm
ttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggacagtgtactaattatgctctcttgaaattggctggagatgttgagagcaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttag

SEQ ID NO: 39 - DNA, 548 bp, Artificial Sequence
  T2A-AscI-yCD2 of pAC3-T2A-yCD2
gagggcagaggaagtcttctaacatgcggtgacgtggaggagaatcccggccctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgc

SEQ ID NO: 40 - DNA, 551 bp, Artificial Sequence
  P2A-AscI-yCD2 of pAC3-P2A-yCD2
gctactaacttcagcctgctgaagcaggctggagacgtggaggagaaccctggacctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgc

SEQ ID NO: 41 - DNA, 557 bp, Artificial Sequence
  GSG-T2A-AscI-yCD2 of pAC3-GSG-T2A-yCD2
ggaagcggagagggcagaggaagtcttctaacatgcggtgacgtggaggagaatcccggccctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgc

SEQ ID NO: 42 - DNA, 560 bp, Artificial Sequence
  GSG-P2A-AscI-yCD2 of pAC3-GSG-P2A-yCD2
ggaagcggagctactaacttcagcctgctgaagcaggctggagacgtggaggagaaccctggacctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgc

SEQ ID NO: 43 - DNA, 11642 bp, Artificial Sequence
  pAC3-T2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagagggcagaggaagtcttctaacatgcggtgacgtggaggagaatcccggccctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 44 - DNA, 11651 bp, Artificial Sequence
  pAC3-GSG-T2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagagggcagaggaagtcttctaacatgcggtgacgtggaggagaatcccggccctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 45 - DNA, 11645 bp, Artificial Sequence
  pAC3-P2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagctactaacttcagcctgctgaagcaggctggagacgtggaggagaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 46 - DNA, 11654 bp, Artificial Sequence
  pAC3-GSG-P2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagctactaacttcagcctgctgaagcaggctggagacgtggaggagaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 47 - DNA, 11648 bp, Artificial Sequence
  pAC3-E2A-GFP
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccacagtgtactaattatgctctcttgaaattggctggagatgttgagagcaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 48 - DNA, 11657 bp, Artificial Sequence
  pAC3-GSG-E2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggacagtgtactaattatgctctcttgaaattggctggagatgttgagagcaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 49 - DNA, 11654 bp, Artificial Sequence; identical to SEQ ID NO: 2
  pAC3-F2A-GFPm

SEQ ID NO: 50 - DNA, 11663 bp, Artificial Sequence
  pAC3-GSG-F2A-GFPm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagtgaaacagactttgaattttgaccttctcaagttggcgggagacgtggagtccaaccctggacctggcgcgcctatggccagcaagggcgaggagctgttcaccggggtggtgcccatcctggtcgagctggacggcgacgtaaacggccacaagttcagcgtgtccggcgaaggagagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgtgcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 51 - DNA, 11399 bp, Artificial Sequence
  pAC3-T2A-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagagggcagaggaagtcttctaacatgcggtgacgtggaggagaatcccggccctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 52 - DNA, 11408 bp, Artificial Sequence
  pAC3-GSG-T2A-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagagggcagaggaagtcttctaacatgcggtgacgtggaggagaatcccggccctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 53 - DNA, 11402 bp, Artificial Sequence
  pAC3-P2A-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccagctactaacttcagcctgctgaagcaggctggagacgtggaggagaaccctggacctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 54 - DNA, 11411 bp, Artificial Sequence
  pAC3-GSG-P2A-yCD2
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagcccaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggatcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccaggaagcggagctactaacttcagcctgctgaagcaggctggagacgtggaggagaaccctggacctggcgcgcctatggtgaccggcggcatggcctccaagtgggatcaaaagggcatggatatcgcttacgaggaggccctgctgggctacaaggagggcggcgtgcctatcggcggctgtctgatcaacaacaaggacggcagtgtgctgggcaggggccacaacatgaggttccagaagggctccgccaccctgcacggcgagatctccaccctggagaactgtggcaggctggagggcaaggtgtacaaggacaccaccctgtacaccaccctgtccccttgtgacatgtgtaccggcgctatcatcatgtacggcatccctaggtgtgtgatcggcgagaacgtgaacttcaagtccaagggcgagaagtacctgcaaaccaggggccacgaggtggtggttgttgacgatgagaggtgtaagaagctgatgaagcagttcatcgacgagaggcctcaggactggttcgaggatatcggcgagtaagcggccgcagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcat

SEQ ID NO: 55 - protein, 20 aa, Artificial Sequence
  Equine rhinitis A virus 2A peptide
QCTNYALLKLAGDVESNPGP

SEQ ID NO: 56 - protein, 23 aa, Artificial Sequence
  Foot-and-mouth disease 2A peptide
PVKQLLNFDLLKLAGDVESNPGP

SEQ ID NO: 57 - protein, 19 aa, Artificial Sequence
  Porcine teschovirus-1 2A peptide
ATNFSLLKQAGDVEENPGP

SEQ ID NO: 58 - protein, 18 aa, Artificial Sequence
  Thosea asigna virus 2A peptide
EGRGSLLTCGDVEENPGP

SEQ ID NO: 59 - protein, 25 aa, Artificial Sequence
  Encephalomyocarditis virus-B 2A peptide
GIFNAHYAGYFADLLIHDIETNPGP

SEQ ID NO: 60 - protein, 17 aa, Artificial Sequence
  Encephalomyocarditis virus-D 2A peptide
GYFADLLIHDIETNPGP

SEQ ID NO: 61 - protein, 25 aa, Artificial Sequence
  Encephalomyocarditis virus-PV21 2A peptide
RIFNAHYAGYFADLLIHDIETNPGP

SEQ ID NO: 62 - protein, 25 aa, Artificial Sequence
  Mengovirus 2A peptide
HVFETHYAGYFSKLLIHDVETNPGP

SEQ ID NO: 63 - protein, 25 aa, Artificial Sequence
  Theiler′s encephalomyelitis virus-GD7 2A peptide
KAVRGYHADYYKQRLIHDVEMNPGP

SEQ ID NO: 64 - protein, 25 aa, Artificial Sequence
  Thieler′s encephalomyelitis virus-DA 2A peptide
RAVRAYHADYYKQRLIHDVEMNPGP

SEQ ID NO: 65 - protein, 25 aa, Artificial Sequence
  Thieler′s encephalomyelitis virus-BEAN 2A peptide
KAVRGYHADYYRQRLIHDVETNPGP

SEQ ID NO: 66 - protein, 25 aa, Artificial Sequence
  Theiler′s-Like Virus 2A peptide
KHVREYHAAYYKQRLMHDVETNPGP

SEQ ID NO: 67 - protein, 26 aa, Artificial Sequence
  Ljungan virus-174F 2A peptide
MHSDEMDFAGGKFLNQCGDVETNPGP

SEQ ID NO: 68 - protein, 26 aa, Artificial Sequence
  Ljungan virus-145SL 2A peptide
MHNDEMDYSGGKFLNQCGDVESNPGP

SEQ ID NO: 69 - protein, 26 aa, Artificial Sequence
  Ljungan virus-(87-012) 2A peptide
MHSDEMDFAGGKFLNQCGDVETNPGP

SEQ ID NO: 70 - protein, 26 aa, Artificial Sequence
  Ljungan Virus - (M1146) 2A peptide
YHDKDMDYAGGKFLNQCGDVETNPGP

SEQ ID NO: 71 - protein, 24 aa, Artificial Sequence
  Foot and Mouth Disease Virus 2A Peptide
APAKQLLNFDLLKLAGDVESNPGP

SEQ ID NO: 72 - protein, 24 aa, Artificial Sequence
  Foot and Mouth Disease Virus-A12 2A Peptide
APGKQLLNFDLLKLAGDVESNPGP

SEQ ID NO: 73 - protein, 24 aa, Artificial Sequence
  Foot and Mouth Disease Virus-C1 2A Peptide
APAKQLLNFDLLKLAGDVESNPGP

SEQ ID NO: 74 - protein, 24 aa, Artificial Sequence
  Foot and Mouth Disease Virus-O1G 2A Peptide
APVKQLLNFDLLKLAGDMESNPGP

SEQ ID NO: 75 - protein, 24 aa, Artificial Sequence
  Foot and Mouth Disease Virus O1K 2A Peptide
APVKQLTNFDLLKLAGDVESNPGP

SEQ ID NO: 76 - protein, 24 aa, Artificial Sequence
  Foot and Mouth Disease Virus - O (Taiwan) 2A Peptide
APAKQLLNFDLLKLAGDVESNPGP

SEQ ID NO: 77 - protein, 24 aa, Artificial Sequence
  Foot and Mouth Disease Virus - O/SK 2A Peptide
APVKQLLSFDLLKLAGDVESNPGP

SEQ ID NO: 78 - protein, 24 aa, Artificial Sequence
  Foot and Mouth Disease Virus - SAT3 2A Peptide
KPDKQMCNFDLLKLAGDVESNPGP

SEQ ID NO: 79 - protein, 24 aa, Artificial Sequence
  Foot and Mouth Disease Virus - SAT2 2A Peptide
GVAKQLLNFDLLKLAGDVESNPGP

SEQ ID NO: 80 - protein, 24 aa, Artificial Sequence
  Equine Rhinits A Virus 2A Peptide
NINKQCTNYSLLKLAGDVESNPGP

SEQ ID NO: 81 - protein, 25 aa, Artificial Sequence
  Equine Rhinitis B Virus 2A Peptide
TILSEGATNFSLLKLAGDVELNPGP

SEQ ID NO: 82 - protein, 25 aa, Artificial Sequence
  Endogenous Retrovirus-3 2A Peptide
NLLSQGATNFDLLKLAGDVESNPGP

SEQ ID NO: 83 - protein, 27 aa, Artificial Sequence
  Punta Toro Virus-1 2A Peptide
VMAFQGPGATNFSLLKQAGDVEENPGP

SEQ ID NO: 84 - protein, 27 aa, Artificial Sequence
  Punta Toro Virus-2 2A Peptide
TMMLQGPGATNFSLLKQAGDVEENPGP

SEQ ID NO: 85 - protein, 27 aa, Artificial Sequence
  Punta Toro Virus-3 2A Peptide
TMSFQGPGASSFSLLKQAGDVEENPGP

SEQ ID NO: 86 - protein, 27 aa, Artificial Sequence
  Punta Toro Virus-4 2A Peptide
TMMLQGPGASNFSLLKQAGDVEENPGP

SEQ ID NO: 87 - protein, 27 aa, Artificial Sequence
  Punta Toro Virus-5 2A Peptide
TMLFQGPGAANFSLLRQAGDVEENPGP

SEQ ID NO: 88 - protein, 27 aa, Artificial Sequence
  Punta Toro Virus-6 2A Peptide
TMSFQGPGATNFSLLKQAGDVEENPGP

SEQ ID NO: 89 - protein, 27 aa, Artificial Sequence
  Punta Toro Virus-7 2A Peptide
VVSFQGPGATNFSLLKQAGDVEENPGP

SEQ ID NO: 90 - protein, 27 aa, Artificial Sequence
  Punta Toro Virus-8 2A Peptide
TMSLQGPGATNFSLLKQAGDIEENPGP

SEQ ID NO: 91 - protein, 27 aa, Artificial Sequence
  Punta Toro Virus-9 2A Peptide
TMAFQGPGATNFSLLKQAGDVEENPGP

SEQ ID NO: 92 - protein, 27 aa, Artificial Sequence
  Punta Toro Virus-10 2A Peptide
TLSFQGPGATNFSLLKQAGDVEENPGP

SEQ ID NO: 93 - protein, 27 aa, Artificial Sequence
  Punta Toro Virus-11 2A Peptide
RMSFQGPGATNFSLLKRAGDVEENPGP

SEQ ID NO: 94 - protein, 20 aa, Artificial Sequence
  Ciricket Paralysis Virus 2A Peptide
FLRKRTQLLMSGDVESNPGP

SEQ ID NO: 95 - protein, 20 aa, Artificial Sequence
  Drosophila C Virus 2A Peptide
EAARQMLLLLSGDVETNPGP

SEQ ID NO: 96 - protein, 20 aa, Artificial Sequence
  Acute Bee Paralysis Virus 2A Peptide
GSWTDILLLLSGDVETNPGP

SEQ ID NO: 97 - protein, 20 aa, Artificial Sequence
  Acute Bee Paralysis Virus Poland 1 isolate 2A Peptide
GSWTDILLLLSGDVETNPGP

SEQ ID NO: 98 - protein, 20 aa, Artificial Sequence
  Acute Bee Paralysis Virus Hungary 1 isolate 2A Peptide
GSWTDILLLWSGDVETNPGP

SEQ ID NO: 99 - protein, 20 aa, Artificial Sequence
  Infectious Flacherie Virus 2A Peptide
TRAEIEDELIRAGIESNPGP

SEQ ID NO: 100 - protein, 20 aa, Artificial Sequence
  Tomato Aspermy Virus 2A Peptide
RAEGRGSLLTCGDVEENPGP

SEQ ID NO: 101 - protein, 20 aa, Artificial Sequence
  Equine Encephalosis Virus 2A Peptide
QGAGRGSLVTCGDVEENPGP

SEQ ID NO: 102 - protein, 20 aa, Artificial Sequence
  Avian Polyoma Virus 2A Peptide
NYPMPEALQKIIDLESNPPP

SEQ ID NO: 103 - protein, 20 aa, Artificial Sequence
  Kashmir bee virus 2A Peptide
GTWESVLNLLAGDIELNPGP

SEQ ID NO: 104 - protein, 20 aa, Artificial Sequence
  Perina Nuda Picorna-like Virus (a) 2A Peptide
AQGWVPDLTVDGDVESNPGP

SEQ ID NO: 105 - protein, 20 aa, Artificial Sequence
  Perina Nuda Picorna-like Virus (b) 2A Peptide
IGGGQKDLTQDGDIESNPGP

SEQ ID NO: 106 - protein, 20 aa, Artificial Sequence
  Ectropis Obliqua Picorna-like Virus (a) 2A Peptide
AQGWAPDLTQDGDVESNPGP

SEQ ID NO: 107 - protein, 20 aa, Artificial Sequence
  Ectropis Obliqua Picorna-like Virus (b) 2A Peptide
IGGGQRDLTQDGDIESNPGP

SEQ ID NO: 108 - protein, 19 aa, Artificial Sequence
  Providence Virus (a) 2A Peptide
VGDRGSLLTCGDVESNPGP

SEQ ID NO: 109 - protein, 19 aa, Artificial Sequence
  Providence Virus (b) 2A Peptide
GDPIEDLTDDGDIEKNPGP

SEQ ID NO: 110 - protein, 19 aa, Artificial Sequence
  Providence Virus (c) 2A Peptide
SGGRGSLLTAGDVEKNPGP

SEQ ID NO: 111 - protein, 20 aa, Artificial Sequence
  Bovine Rotavirus 2A Peptide
SKFQIDRILISGDIELNPGP

SEQ ID NO: 112 - protein, 20 aa, Artificial Sequence
  Porcine Rotavirus 2A Peptide
AKFQIDKILISGDVELNPGP

SEQ ID NO: 113 - protein, 20 aa, Artificial Sequence
  Human Rotavirus 2A Peptide
SKFQIDKILISGDIELNPGP

SEQ ID NO: 114 - protein, 20 aa, Artificial Sequence
  Bombyx Mori Reovirus 2A Peptide
FRSNYDLLKLCGDIESNPGP

SEQ ID NO: 115 - protein, 20 aa, Artificial Sequence
  Lymantria Dispar Reovirus 2A Peptide
FRSNYDLLKLCGDVESNPGP

SEQ ID NO: 116 - protein, 20 aa, Artificial Sequence
  Dendrolimus Punctatus Reovirus 2A Peptide
FRSNYDLLKLCGDVESNPGP

SEQ ID NO: 117 - protein, 20 aa, Artificial Sequence
  Trypansoma Brucei TSR1 2A Peptide
SSIIRTKMLVSGDVEENPGP

SEQ ID NO: 118 - protein, 20 aa, Artificial Sequence
  Trypansoma Spp. CAB95325.1 2A Peptide
SSIIRTKMLLSGDVEENPGP

SEQ ID NO: 119 - protein, 20 aa, Artificial Sequence
  Trypansoma Spp. CAB95559.1 2A Peptide
SSIIRTKILLSGDVEENPGP

SEQ ID NO: 120 - protein, 20 aa, Artificial Sequence
  Trypansoma Cruzi 2A Peptide
CDAQRQKLLLSGDIEQNPGP

SEQ ID NO: 121 - protein, 20 aa, Artificial Sequence
  T. maritima aguA 2A Peptide
YIPDFGGFLVKADSEFNPGP

SEQ ID NO: 122 - protein, 21 aa, Artificial Sequence
  B. bronchiseptica 2A Peptide
VHCAGRGGPVRLLDKEGNPGP

SEQ ID NO: 123 - protein, 20 aa, Artificial Sequence
  Murine mor-1F 2A Peptide
DLELETVGSHQADAETNPGP

SEQ ID NO: 124 - protein, 20 aa, Artificial Sequence
  D. melanogaster mod(mdg4) 2A Peptide
TAADKIQGSWKMDTEGNPGP

SEQ ID NO: 125 - protein, 20 aa, Artificial Sequence
  A. nidulans Ca Channel MID1 2A Peptide
PITNRPRNSGLIDTEINPGP

SEQ ID NO: 126 - DNA, 288 bp, Artificial Sequence
  Adnectins (10Fn3) sequence
  CDS (1)..(288)
gtgagcgacgtgcccagaaagctggaggtggtggccgccacccccaccagcctgctgatcagctgggacgcccccgccgtgaccgtgagatactacagaatcacctacggcgagaccggcggcaacagccccgtgcaggagttcaccgtgcccggcagcaagagcaccgccaccatcagcggcctgaagcccggcgtggactacaccatcaccgtgtacgccgtgaccggcagaggcgacagccccgccagcagcaagcccatcagcaactacagaaccgccctggag

SEQ ID NO: 127 - protein, 96 aa, Artificial Sequence
  Synthetic Construct
VSDVPRKLEVVAATPTSLLISWDAPAVTVRYYRITYGETGGNSPVQEFTVPGSKSTATISGLKPGVDYTITVYAVTGRGDSPASSKPISNYRTALE

SEQ ID NO: 128 - DNA, 303 bp, Artificial Sequence
  Adnectin 1
  CDS (1)..(303)
gtgagcgacgtgcccagaaagctggaggtggtggccgccacccccaccagcctgctgatcagctgggacagcggcagaggcagctacagatactacagaatcacctacggcgagaccggcggcaacagccccgtgcaggagttcaccgtgcccggccccgtgcacaccgccaccatcagcggcctgaagcccggcgtggactacaccatcaccgtgtacgccgtgaccgaccacaagccccacgccgacggcccccacacctaccacgagagccccatcagcaactacagaaccgccctggag

SEQ ID NO: 129 - protein, 101 aa, Artificial Sequence
  Synthetic Construct
VSDVPRKLEVVAATPTSLLISWDSGRGSYRYYRITYGETGGNSPVQEFTVPGPVHTATISGLKPGVDYTITVYAVTDHKPHADGPHTYHESPISNYRTALE

SEQ ID NO: 130 - DNA, 288 bp, Artificial Sequence
  Adnectin 2
  CDS (1)..(288)
gtgagcgacgtgcccagaaagctggaggtggtggccgccacccccaccagcctgctgatcagctgggagcacgactacccctacagaagatactacagaatcacctacggcgagaccggcggcaacagccccgtgcaggagttcaccgtgcccaaggacgtggacaccgccaccatcagcggcctgaagcccggcgtggactacaccatcaccgtgtacgccgtgaccagcagctacaagtacgacatgcagtacagccccatcagcaactacagaaccgccctggag

SEQ ID NO: 131 - protein, 96 aa, Artificial Sequence
  Synthetic Construct
VSDVPRKLEVVAATPTSLLISWEHDYPYRRYYRITYGETGGNSPVQEFTVPKDVDTATISGLKPGVDYTITVYAVTSSYKYDMQYSPISNYRTALE

SEQ ID NO: 132 - DNA, 276 bp, Artificial Sequence
  Pronectins (1Fn3)
  CDS (1)..(276)
agcggccccgtggaggtgttcatcaccgagacccccagccagcccaacagccaccccatccagtggaacgccccccagcccagccacatcagcaagtacatcctgagatggagacccaagaacagcgtgggcagatggaaggaggccaccatccccggccacctgaacagctacaccatcaagggcctgaagcccggcgtggtgtacgagggccagctgatcagcatccagcagtacggccaccaggaggtgaccagattcgacttcaccaccacc

SEQ ID NO: 133 - protein, 92 aa, Artificial Sequence
  Synthetic Construct
SGPVEVFITETPSQPNSHPIQWNAPQPSHISKYILRWRPKNSVGRWKEATIPGHLNSYTIKGLKPGVVYEGQLISIQQYGHQEVTRFDFTTT

SEQ ID NO: 134 - DNA, 270 bp, Artificial Sequence
  Pronectins (2Fn3)
  CDS (1)..(270)
agccccctggtggccaccagcgagagcgtgaccgagatcaccgccagcagcttcgtggtgagctgggtgagcgccagcgacaccgtgagcggcttcagagtggagtacgagctgagcgaggagggcgacgagccccagtacctggacctgcccagcaccgccaccagcgtgaacatccccgacctgctgcccggcagaaagtacatcgtgaacgtgtaccagagcgaggacggcgagcagagcctgatcctgagcaccagccagaccacc

SEQ ID NO: 135 - protein, 90 aa, Artificial Sequence
  Synthetic Construct
SPLVATSESVTEITASSFVVSWVSASDTVSGFRVEYELSEEGDEPQYLDLPSTATSVNIPDLLPGRKYIVNVYQSEDGEQSLILSTSQTT

SEQ ID NO: 136 - DNA, 282 bp, Artificial Sequence
  Pronectins (3Fn3)
  CDS (1)..(282)
gcccccgacgccccccccgaccccaccgtggaccaggtggacgacaccagcatcgtggtgagatggagcagaccccaggcccccatcaccggctacagaatcgtgtacagccccagcgtggagggcagcagcaccgagctgaacctgcccgagaccgccaacagcgtgaccctgagcgacctgcagcccggcgtgcagtacaacatcaccatctacgccgtggaggagaaccaggagagcacccccgtggtgatccagcaggagaccaccggcacccccaga

SEQ ID NO: 137 - protein, 94 aa, Artificial Sequence
  Synthetic Construct
APDAPPDPTVDQVDDTSIVVRWSRPQAPITGYRIVYSPSVEGSSTELNLPETANSVTLSDLQPGVQYNITIYAVEENQESTPVVIQQETTGTPR

SEQ ID NO: 138 - DNA, 270 bp, Artificial Sequence
  Pronectins (4Fn3)
  CDS (1)..(270)
accgtgcccagccccagagacctgcagttcgtggaggtgaccgacgtgaaggtgaccatcatgtggaccccccccgagagcgccgtgaccggctacagagtggacgtgatccccgtgaacctgcccggcgagcacggccagagactgcccatcagcagaaacaccttcgccgaggtgaccggcctgagccccggcgtgacctactacttcaaggtgttcgccgtgagccacggcagagagagcaagcccctgaccgcccagcagaccacc

SEQ ID NO: 139 - protein, 90 aa, Artificial Sequence
  Synthetic Construct
TVPSPRDLQFVEVTDVKVTIMWTPPESAVTGYRVDVIPVNLPGEHGQRLPISRNTFAEVTGLSPGVTYYFKVFAVSHGRESKPLTAQQTT

SEQ ID NO: 140 - DNA, 270 bp, Artificial Sequence
  Pronectins (5Fn3)
  CDS (1)..(270)
aagctggacgcccccaccaacctgcagttcgtgaacgagaccgacagcaccgtgctggtgagatggaccccccccagagcccagatcaccggctacagactgaccgtgggcctgaccagaagaggccagcccagacagtacaacgtgggccccagcgtgagcaagtaccccctgagaaacctgcagcccgccagcgagtacaccgtgagcctggtggccatcaagggcaaccaggagagccccaaggccaccggcgtgttcaccaccctg

SEQ ID NO: 141 - protein, 90 aa, Artificial Sequence
  Synthetic Construct
KLDAPTNLQFVNETDSTVLVRWTPPRAQITGYRLTVGLTRRGQPRQYNVGPSVSKYPLRNLQPASEYTVSLVAIKGNQESPKATGVFTTL

SEQ ID NO: 142 - DNA, 258 bp, Artificial Sequence
  Pronectins (6Fn3)
  CDS (1)..(258)
cagcccggcagcagcatccccccctacaacaccgaggtgaccgagaccaccatcgtgatcacctggacccccgcccccagactgggcttcaagctgggcgtgagacccagccagggcggcgaggcccccagagaggtgaccagcgacagcggcagcgtggtgagcggcctgacccccggcgtggagtacgtgtacaccatccaggtgctgagagacggccaggagagagacgcccccatcgtgaacaaggtggtgacc

SEQ ID NO: 143 - protein, 86 aa, Artificial Sequence
  Synthetic Construct
QPGSSIPPYNTEVTETTIVITWTPAPRLGFKLGVRPSQGGEAPREVTSDSGSVVSGLTPGVEYVYTIQVLRDGQERDAPIVNKVVT

SEQ ID NO: 144 - DNA, 282 bp, Artificial Sequence
  Pronectins (7Fn3)
  CDS (1)..(282)
cccctgagcccccccaccaacctgcacctggaggccaaccccgacaccggcgtgctgaccgtgagctgggagagaagcaccacccccgacatcaccggctacagaatcaccaccacccccaccaacggccagcagggcaacagcctggaggaggtggtgcacgccgaccagagcagctgcaccttcgacaacctgagccccggcctggagtacaacgtgagcgtgtacaccgtgaaggacgacaaggagagcgtgcccatcagcgacaccatcatcccctga

SEQ ID NO: 145 - protein, 93 aa, Artificial Sequence
  Synthetic Construct
PLSPPTNLHLEANPDTGVLTVSWERSTTPDITGYRITTTPTNGQQGNSLEEVVHADQSSCTFDNLSPGLEYNVSVYTVKDDKESVPISDTIIP

SEQ ID NO: 146 - DNA, 276 bp, Artificial Sequence
  Pronectins (8Fn3)
  CDS (1)..(276)
gccgtgcccccccccaccgacctgagattcaccaacatcggccccgacaccatgagagtgacctgggcccccccccccagcatcgacctgaccaacttcctggtgagatacagccccgtgaagaacgaggaggacgtggccgagctgagcatcagccccagcgacaacgccgtggtgctgaccaacctgctgcccggcaccgagtacgtggtgagcgtgagcagcgtgtacgagcagcacgagagcacccccctgagaggcagacagaagacctga

SEQ ID NO: 147 - protein, 91 aa, Artificial Sequence
  Synthetic Construct
AVPPPTDLRFTNIGPDTMRVTWAPPPSIDLTNFLVRYSPVKNEEDVAELSISPSDNAVVLTNLLPGTEYVVSVSSVYEQHESTPLRGRQKT

SEQ ID NO: 148 - DNA, 273 bp, Artificial Sequence
  Pronectins (9Fn3)
  CDS (1)..(273)
ggcctggacagccccaccggcatcgacttcagcgacatcaccgccaacagcttcaccgtgcactggatcgcccccagagccaccatcaccggctacagaatcagacaccaccccgagcacttcagcggcagacccagagaggacagagtgccccacagcagaaacagcatcaccctgaccaacctgacccccggcaccgagtacgtggtgagcatcgtggccctgaacggcagagaggagagccccctgctgatcggccagcagagcacctga

SEQ ID NO: 149 - protein, 90 aa, Artificial Sequence
  Synthetic Construct
GLDSPTGIDFSDITANSFTVHWIAPRATITGYRIRHHPEHFSGRPREDRVPHSRNSITLTNLTPGTEYVVSIVALNGREESPLLIGQQST

SEQ ID NO: 150 - DNA, 279 bp, Artificial Sequence
  Pronectins (10Fn3)
  CDS (1)..(279)
gtgagcgacgtgcccagagacctggtggtggccgccacccccaccagcctgctgatcagctgggacgcccccgccgtgaccgtgagatactacagaatcacctacggcgagaccggcggcaacagccccgtgcaggagttcaccgtgcccggcagcaagagcaccgccaccatcagcggcctgaagcccggcgtggactacaccatcaccgtgtacgccgtgaccggcagaggcgacagccccgccagcagcaagcccatcagcatcaactacagaacc

SEQ ID NO: 151 - protein, 93 aa, Artificial Sequence
  Synthetic Construct
VSDVPRDLVVAATPTSLLISWDAPAVTVRYYRITYGETGGNSPVQEFTVPGSKSTATISGLKPGVDYTITVYAVTGRGDSPASSKPISINYRT

SEQ ID NO: 152 - DNA, 270 bp, Artificial Sequence
  Pronectins (11Fn3)
  CDS (1)..(270)
gagatcgacaagcccagccagatgcaggtgaccgacgtgcaggacaacagcatcagcgtgaagtggctgcccagcagcagccccgtgaccggctacagagtgaccaccacccccaagaacggccccggccccaccaagaccaagaccgccggccccgaccagaccgagatgaccatcgagggcctgcagcccaccgtggagtacgtggtgagcgtgtacgcccagaaccccagcggcgagagccagcccctggtgcagaccgccgtgacc

SEQ ID NO: 153 - protein, 90 aa, Artificial Sequence
  Synthetic Construct
EIDKPSQMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 154 - DNA, 273 bp, Artificial Sequence
  Pronectins (12Fn3)
  CDS (1)..(273)
aacatcgacagacccaagggcctggccttcaccgacgtggacgtggacagcatcaagatcgcctgggagagcccccagggccaggtgagcagatacagagtgacctacagcagccccgaggacggcatccacgagctgttccccgcccccgacggcgaggaggacaccgccgagctgcagggcctgagacccggcagcgagtacaccgtgagcgtggtggccctgcacgacgacatggagagccagcccctgatcggcacccagagcacctga

SEQ ID NO: 155 - protein, 90 aa, Artificial Sequence
  Synthetic Construct
NIDRPKGLAFTDVDVDSIKIAWESPQGQVSRYRVTYSSPEDGIHELFPAPDGEEDTAELQGLRPGSEYTVSVVALHDDMESQPLIGTQST

SEQ ID NO: 156 - DNA, 276 bp, Artificial Sequence
  Pronectins (13Fn3)
  CDS (1)..(276)
gccatccccgcccccaccgacctgaagttcacccaggtgacccccaccagcctgagcgcccagtggaccccccccaacgtgcagctgaccggctacagagtgagagtgacccccaaggagaagaccggccccatgaaggagatcaacctggcccccgacagcagcagcgtggtggtgagcggcctgatggtggccaccaagtacgaggtgagcgtgtacgccctgaaggacaccctgaccagcagacccgcccagggcgtggtgaccaccctggag

SEQ ID NO: 157 - protein, 92 aa, Artificial Sequence
  Synthetic Construct
AIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 158 - DNA, 264 bp, Artificial Sequence
  Pronectins (14Fn3)
  CDS (1)..(264)
aacgtgagcccccccagaagagccagagtgaccgacgccaccgagaccaccatcaccatcagctggagaaccaagaccgagaccatcaccggcttccaggtggacgccgtgcccgccaacggccagacccccatccagagaaccatcaagcccgacgtgagaagctacaccatcaccggcctgcagcccggcaccgactacaagatctacctgtacaccctgaacgacaacgccagaagcagcgtggtgatcgacgccagcacc

SEQ ID NO: 159 - protein, 88 aa, Artificial Sequence
  Synthetic Construct
NVSPPRRARVTDATETTITISWRTKTETITGFQVDAVPANGQTPIQRTIKPDVRSYTITGLQPGTDYKIYLYTLNDNARSSVVIDAST

SEQ ID NO: 160 - DNA, 270 bp, Artificial Sequence
  Pronectins (15Fn3)
  CDS (1)..(270)
gccatcgacgcccccagcaacctgagattcctggccaccacccccaacagcctgctggtgagctggcagccccccagagccagaatcaccggctacatcatcaagtacgagaagcccggcagcccccccagagaggtggtgcccagacccagacccggcgtgaccgaggccaccatcaccggcctggagcccggcaccgagtacaccatctacgtgatcgccctgaagaacaaccagaagagcgagcccctgatcggcagaaagaagacc

SEQ ID NO: 161 - protein, 90 aa, Artificial Sequence
  Synthetic Construct
AIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 162 - DNA, 264 bp, Artificial Sequence
  Pronectins (16Fn3)
  CDS (1)..(264)
cccggcctgaaccccaacgccagcaccggccaggaggccctgagccagaccaccatcagctgggcccccttccaggacaccagcgagtacatcatcagctgccaccccgtgggcaccgacgaggagcccctgcagttcagagtgcccggcaccagcaccagcgccaccctgaccggcctgaccagaggcgccacctacaacatcatcgtggaggccctgaaggaccagcagagacacaaggtgagagaggaggtggtgaccgtg

SEQ ID NO: 163 - protein, 88 aa, Artificial Sequence
  Synthetic Construct
PGLNPNASTGQEALSQTTISWAPFQDTSEYIISCHPVGTDEEPLQFRVPGTSTSATLTGLTRGATYNIIVEALKDQQRHKVREEVVTV

SEQ ID NO: 164 - DNA, 276 bp, Artificial Sequence
  Adhiron
  CDS (1)..(276)
gccaccggcgtgagagccgtgcccggcaacgagaacagcctggagatcgaggagctggccagattcgccgtggacgagcacaacaagaaggagaacgccctgctggagttcgtgagagtggtgaaggccaaggagcaggtggtggccggcaccatgtactacctgaccctggaggccaaggacggcggcaagaagaagctgtacgaggccaaggtgtgggtgaagccctgggagaacttcaaggagctgcaggagttcaagcccgtgggcgacgcc

SEQ ID NO: 165 - protein, 92 aa, Artificial Sequence
  Synthetic Construct
ATGVRAVPGNENSLEIEELARFAVDEHNKKENALLEFVRVVKAKEQVVAGTMYYLTLEAKDGGKKKLYEAKVWVKPWENFKELQEFKPVGDA

SEQ ID NO: 166 - DNA, 177 bp, Artificial Sequence
  Affibodies
  CDS (1)..(177)
gtggacaacaagttcaacaaggagcagcagaacgccttctacgagatcctgcacctgcccaacctgaacgaggagcagagaaacgccttcatccagagcctgaaggacgaccccagccagagcgccaacctgctggccgaggccaagaagctgaacgacgcccaggcccccaagtga

SEQ ID NO: 167 - protein, 58 aa, Artificial Sequence [sequence omitted]

SEQ ID NO: 168 - DNA, 522 bp, Artificial Sequence
  Affilins (gamma-B Cyrstallin)
  CDS (1)..(522)
ggcaagatcaccttctacgaggacagagccttccagggcagaagctacgagtgcaccaccgactgccccaacctgcagccctacttcagcagatgcaacagcatcagagtggagagcggctgctggatgatctacgagagacccaactaccagggccaccagtacttcctgagaagaggcgagtaccccgactaccagcagtggatgggcctgagcgacagcatcagaagctgctgcctgatccccccccacagcggcgcctacagaatgaagatctacgacagagacgagctgagaggccagatgagcgagctgaccgacgactgcatcagcgtgcaggacagattccacctgaccgagatccacagcctgaacgtgctggagggcagctggatcctgtacgagatgcccaactacagaggcagacagtacctgctgagacccggcgagtacagaagattcctggactggggcgcccccaacgccaaggtgggcagcctgagaagagtgatggacctgtac

SEQ ID NO: 169 - protein, 174 aa, Artificial Sequence
  Synthetic Construct
GKITFYEDRAFQGRSYECTTDCPNLQPYFSRCNSIRVESGCWMIYERPNYQGHQYFLRRGEYPDYQQWMGLSDSIRSCCLIPPHSGAYRMKIYDRDELRGQMSELTDDCISVQDRFHLTEIHSLNVLEGSWILYEMPNYRGRQYLLRPGEYRRFLDWGAPNAKVGSLRRVMDLY

SEQ ID NO: 170 - DNA, 294 bp, Artificial Sequence
  Affimers
  CDS (1)..(294)
atgatccccagaggcctgagcgaggccaagcccgccacccccgagatccaggagatcgtggacaaggtgaagccccagctggaggagaagaccaacgagacctacggcaagctggaggccgtgcagtacaagacccaggtgctggccagcaccaactactacatcaaggtgagagccggcgacaacaagtacatgcacctgaaggtgttcaacggcccccccggccagaacgccgacagagtgctgaccggctaccaggtggacaagaacaaggacgacgagctgaccggcttc

SEQ ID NO: 171 - protein, 98 aa, Artificial Sequence
  Synthetic Construct
MIPRGLSEAKPATPEIQEIVDKVKPQLEEKTNETYGKLEAVQYKTQVLASTNYYIKVRAGDNKYMHLKVFNGPPGQNADRVLTGYQVDKNKDDELTGF

SEQ ID NO: 172 - DNA, 462 bp, Artificial Sequence
  Anticalin (lipocalin Lcn1)
  CDS (1)..(462)
atcgccagcgacgaggagatccaggacgtgagcggcacctggtacctgaaggccatgaccgtggacagagagttccccgagatgaacctggagagcgtgacccccatgaccctgaccaccctggagggcggcaacctggaggccaaggtgaccatgctgatcagcggcagatgccaggaggtgaaggccgtgctggagaagaccgacgagcccggcaagtacaccgccgacggcggcaagcacgtggcctacatcatcagaagccacgtgaaggaccactacatcttctacagcgagggcgagctgcacggcaagcccgtgagaggcgtgaagctggtgggcagagaccccaagaacaacctggaggccctgctggacttcgagaaggccgccggcgccagaggcctgagcaccgagagcatcctgatccccagacagagcgagacctgcagccccggcagc

SEQ ID NO: 173 - protein, 154 aa, Artificial Sequence
  Synthetic Construct
IASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYSEGELHGKPVRGVKLVGRDPKNNLEALLDFEKAAGARGLSTESILIPRQSETCSPGS

SEQ ID NO: 174 - DNA, 534 bp, Artificial Sequence
  Anticalins (lipocalin Lcn2)
  CDS (1)..(534)
caggacagcaccagcgacctgatccccgccccccccctgagcaaggtgcccctgcagcagaacttccaggacaaccagttccagggcaagtggtacgtggtgggcctggccggcaacgccatcctgagagaggacaaggacccccagaagatgtacgccaccatctacgagctgaaggaggacaagagctacaacgtgaccagcgtgctgttcagaaagaagaagtgcgactactggatcagaaccttcgtgcccggctgccagcccggcgagttcaccctgggcaacatcaagagctaccccggcctgaccagctacctggtgagagtggtgagcaccaactacaaccagcacgccatggtgttcttcaagaaggtgagccagaacagagagtacttcaagatcaccctgtacggcagaaccaaggagctgaccagcgagctgaaggagaacttcatcagattcagcaagagcctgggcctgcccgagaaccacatcgtgttccccgtgcccatcgaccagtgcatcgacggc

SEQ ID NO: 175 - protein, 178 aa, Artificial Sequence
  Synthetic Construct
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 176 - DNA, 255 bp, Artificial Sequence
  Avimers (C426) targeting c-MET
  CDS (1)..(255)
tgcgagagcggcgagttccagtgccacagcaccggcagatgcatcccccaggagtgggtgtgcgacggcgacaacgactgcgaggacagcagcgacgaggcccccgacctgtgcgccagcgccgagcccacctgccccagcggcgagttccagtgcagaagcaccaacagatgcatccccgagacctggctgtgcgacggcgacaacgactgcgaggacggcagcgacgaggagagctgcaccccccccacctga

SEQ ID NO: 177 - protein, 84 aa, Artificial Sequence
  Synthetic Construct
CESGEFQCHSTGRCIPQEWVCDGDNDCEDSSDEAPDLCASAEPTCPSGEFQCRSTNRCIPETWLCDGDNDCEDGSDEESCTPPT

SEQ ID NO: 178 - DNA, 267 bp, Artificial Sequence
  Centyrins (Fn3 domain of Tenascin)
  CDS (1)..(267)
ctgcccgcccccaagaacctggtggtgagcgaggtgaccgaggacagcgccagactgagctggaccgcccccgacgccgccttcgacagcttcctgatcggctacggcgagagcgagaaggtgggcgaggccatcgtgctgaccgtgcccggcagcgagagaagctacgacctgaccggcctgaagcccggcaccgagtacaccgtgagcatctacggcgtgaagggcggccacagaagcaaccccctgagcgccatcttcaccacc

SEQ ID NO: 179 - protein, 89 aa, Artificial Sequence
  Synthetic Construct
LPAPKNLVVSEVTEDSARLSWTAPDAAFDSFLIGYGESEKVGEAIVLTVPGSERSYDLTGLKPGTEYTVSIYGVKGGHRSNPLSAIFTT

SEQ ID NO: 180 - DNA, 171 bp, Artificial Sequence
  Kunitz domain/BPTI
  CDS (1)..(171)
gtgagagaggtgtgcagcgagcaggccgagaccggcccctgcagagccatgatcagcagatggtacttcgacgtgaccgagggcaagtgcgcccccttcttctacggcggctgctgcggcggcaacagaaacaacttcgacaccgaggagtactgcatggccgtgtgcggc

SEQ ID NO: 181 - protein, 57 aa, Artificial Sequence
  Synthetic Construct
VREVCSEQAETGPCRAMISRWYFDVTEGKCAPFFYGGCCGGNRNNFDTEEYCMAVCG

SEQ ID NO: 182 - DNA, 516 bp, Artificial Sequence
  Obodies (human AspRS)
  CDS (1)..(516)
gagatcatggacgccgccgaggactacgccaaggagagatacggcatcagcagcatgatccagagccaggagaagcccgacagagtgctggtgagagtgagagacctgaccatccagaaggccgacgaggtggtgtgggtgagagccagagtgcacaccagcagagccaagggcaagcagtgcttcctggtgctgagacagcagcagttcaacgtgcaggccctggtggccgtgggcgaccacgccagcaagcagatggtgaagttcgccgccaacatcaacaaggagagcatcgtggacgtggagggcgtggtgagaaaggtgaaccagaagatcggcagctgcacccagcaggacgtggagctgcacgtgcagaagatctacgtgatcagcctggccgagcccagactgcccctgcagctggacgacgccgtgagacccgaggccgagggcgaggaggagggcagagccaccgtgaaccaggacaccagactggacaacagagtgatcgacctg

SEQ ID NO: 183 - protein, 172 aa, Artificial Sequence
  Synthetic Construct
EIMDAAEDYAKERYGISSMIQSQEKPDRVLVRVRDLTIQKADEVVWVRARVHTSRAKGKQCFLVLRQQQFNVQALVAVGDHASKQMVKFAANINKESIVDVEGVVRKVNQKIGSCTQQDVELHVQKIYVISLAEPRLPLQLDDAVRPEAEGEEEGRATVNQDTRLDNRVIDL

SEQ ID NO: 184 - DNA, 267 bp, Artificial Sequence
  Tn3A
  CDS (1)..(267)
gccatcgaggtgaaggacgtgaccgacaccaccgccctgatcacctggagcgacgagttcggccacgactacgacggctgcgagctgacctacggcatcaaggacgtgcccggcgacagaaccaccatcgacctgtggtggcacagcgcctggtacagcatcggcaacctgaagcccgacaccgaggacgtgagcctgatctgctacaccgaccaggaggccggcaaccccgccaaggagaccttcaccaccggcctggtgcccaga

SEQ ID NO: 185 - protein, 89 aa, Artificial Sequence
  Synthetic Construct
AIEVKDVTDTTALITWSDEFGHDYDGCELTYGIKDVPGDRTTIDLWWHSAWYSIGNLKPDTEDVSLICYTDQEAGNPAKETFTTGLVPR

SEQ ID NO: 186 - DNA, 276 bp, Artificial Sequence
  Tn3B
  CDS (1)..(276)
gccatcgaggtggaggacgtgaccgacaccaccgccctgatcacctggaccaacagaagcagctacagcaacctgcacggctgcgagctggcctacggcatcaaggacgtgcccggcgacagaaccaccatcgacctgaaccagccctacgtgcactacagcatcggcaacctgaagcccgacaccgagtacgaggtgagcctgatctgcctgaccaccgacggcacctacaacaaccccgccaaggagaccttcaccaccggcctggtgcccaga

SEQ ID NO: 187 - protein, 92 aa, Artificial Sequence
  Synthetic Construct
AIEVEDVTDTTALITWTNRSSYSNLHGCELAYGIKDVPGDRTTIDLNQPYVHYSIGNLKPDTEYEVSLICLTTDGTYNNPAKETFTTGLVPR

SEQ ID NO: 188 - DNA, 177 bp, Artificial Sequence
  Hckomers
  CDS (1)..(177)
accctgttcgtggccctgtacgactacgaggccagaaccgaggacgagctgagcttccacaagggcgagaagttccagatcctgaacagcagcgagggcgactggtgggaggccagagacagcctgaccaccggcgagaccggctacatccccagcaactacgtggcccccgtggac

SEQ ID NO: 189 - protein, 59 aa, Artificial Sequence
  Synthetic Construct
TLFVALYDYEARTEDELSFHKGEKFQILNSSEGDWWEARDSLTTGETGYIPSNYVAPVD

SEQ ID NO: 190 - DNA, 174 bp, Artificial Sequence
  NPHP1
  CDS (1)..(174)
gaggagtacatcgccgtgggcgacttcgacaccgcccagcaggtgggcgacctgaccttcaagaagggcgagatcctgctggtgatcgagaagaagcccgacggctggtggatcgccaaggacgccaagggcaacgagggcctggtgcccagaacctacctggagccctacagc

SEQ ID NO: 191 - protein, 58 aa, Artificial Sequence
  Synthetic Construct
EEYIAVGDFDTAQQVGDLTFKKGEILLVIEKKPDGWWIAKDAKGNEGLVPRTYLEPYS

SEQ ID NO: 192 - DNA, 171 bp, Artificial Sequence
  Tec
  CDS (1)..(171)
gagatcgtggtggccatgtacgacttccaggccgccgagggccacgacctgagactggagagacaggagtacctgatcctggagaagaacgacgtgcactggtggagagccagagacaagtacggcaacgagggctacatccccagcaactacgtgaccggcaagaagtga

SEQ ID NO: 193 - protein, 56 aa, Artificial Sequence
  Synthetic Construct
EIVVAMYDFQAAEGHDLRLERQEYLILEKNDVHWWRARDKYGNEGYIPSNYVTGKK

SEQ ID NO: 194 - DNA, 171 bp, Artificial sequence
  Hck
  CDS (1)..(171)
atcatcgtggtggccctgtacgactacgaggccatccaccacgaggacctgagcttccagaagggcgaccagatggtggtgctggaggagagcggcgagtggtggaaggccagaagcctggccaccagaaaggagggctacatccccagcaactacgtggccagagtggac

SEQ ID NO: 195 - protein, 57 aa, Artificial sequence
  Synthetic Construct
IIVVALYDYEAIHHEDLSFQKGDQMVVLEESGEWWKARSLATRKEGYIPSNYVARVD

SEQ ID NO: 196 - DNA, 213 bp, Artificial Sequence
  Amph
  CDS (1)..(213)
tacaaggtggagaccctgcacgacttcgaggccgccaacagcgacgagctgaccctgcagagaggcgacgtggtgctggtggtgcccagcgacagcgaggccgaccaggacgccggctggctggtgggcgtgaaggagagcgactggctgcagtacagagacctggccacctacaagggcctgttccccgagaacttcaccagaagactggac

SEQ ID NO: 197 - protein, 71 aa, Artificial Sequence
  Synthetic Construct
YKVETLHDFEAANSDELTLQRGDVVLVVPSDSEADQDAGWLVGVKESDWLQYRDLATYKGLFPENFTRRLD

SEQ ID NO: 198 - DNA, 192 bp, Artificial Sequence
  RIMBP#3
  CDS (1)..(192)
aagatcatgatcgccgccctggactacgaccccggcgacggccagatgggcggccagggcaagggcagactggccctgagagccggcgacgtggtgatggtgtacggccccatggacgaccagggcttctactacggcgagctgggcggccacagaggcctggtgcccgcccacctgctggaccacatgagc

SEQ ID NO: 199 - protein, 64 aa, Artificial Sequence
  Synthetic Construct
KIMIAALDYDPGDGQMGGQGKGRLALRAGDVVMVYGPMDDQGFYYGELGGHRGLVPAHLLDHMS

SEQ ID NO: 200 - DNA, 180 bp, Artificial Sequence
  IRIKS
  CDS (1)..(180)
cagaaggtgaagaccatcttcccccacaccgccggcagcaacaagaccctgctgagcttcgcccagggcgacgtgatcaccctgctgatccccgaggagaaggacggctggctgtacggcgagcacgacgtgagcaaggccagaggctggttccccagcagctacaccaagctgctggag

SEQ ID NO: 201 - protein, 60 aa, Artificial Sequence
  Synthetic Construct
QKVKTIFPHTAGSNKTLLSFAQGDVITLLIPEEKDGWLYGEHDVSKARGWFPSSYTKLLE

SEQ ID NO: 202 - DNA, 174 bp, Artificial Sequence
  SNX33
  CDS (1)..(174)
ctgaagggcagagccctgtacgacttccacagcgagaacaaggaggagatcagcatccagcaggacgaggacctggtgatcttcagcgagaccagcctggacggctggctgcagggccagaacagcagaggcgagaccggcctgttccccgccagctacgtggagatcgtgaga

SEQ ID NO: 203 - protein, 58 aa, Artificial Sequence
  Synthetic Construct
LKGRALYDFHSENKEEISIQQDEDLVIFSETSLDGWLQGQNSRGETGLFPASYVEIVR

SEQ ID NO: 204 - DNA, 168 bp, Artificial Sequence
  Eps8L1
  CDS (1)..(168)
aagtgggtgctgtgcaactacgacttccaggccagaaacagcagcgagctgagcgtgaagcagagagacgtgctggaggtgctggacgacagcagaaagtggtggaaggtgagagaccccgccggccaggagggctacgtgccctacaacatcctgaccccctacccc

SEQ ID NO: 205 - protein, 56 aa, Artificial Sequence
  Synthetic Construct
KWVLCNYDFQARNSSELSVKQRDVLEVLDDSRKWWKVRDPAGQEGYVPYNILTPYP

SEQ ID NO: 206 - DNA, 177 bp, Artificial Sequence
  FISH#5
  CDS (1)..(177)
gacgtgtacgtgagcatcgccgactacgagggcgacgaggagaccgccggcttccaggagggcgtgagcatggaggtgctggagagaaaccccaacggctggtggtactgccagatcctggacggcgtgaagcccttcaagggctgggtgcccagcaactacctggagaagaagaac

SEQ ID NO: 207 - protein, 59 aa, Artificial Sequence
  Synthetic Construct
DVYVSIADYEGDEETAGFQEGVSMEVLERNPNGWWYCQILDGVKPFKGWVPSNYLEKKN

SEQ ID NO: 208 - DNA, 171 bp, Artificial Sequence
  CMS#1
  CDS (1)..(171)
gtggactacatcgtggagtacgactacgacgccgtgcacgacgacgagctgaccatcagagtgggcgagatcatcagaaacgtgaagaagctgcaggaggagggctggctggagggcgagctgaacggcagaagaggcatgttccccgacaacttcgtgaaggagatcaag

SEQ ID NO: 209 - protein, 57 aa, Artificial Sequence
  Synthetic Construct
VDYIVEYDYDAVHDDELTIRVGEIIRNVKKLQEEGWLEGELNGRRGMFPDNFVKEIK

SEQ ID NO: 210 - DNA, 168 bp, Artificial Sequence
  OSTF1
  CDS (1)..(168)
aaggtgttcagagccctgtacaccttcgagcccagaacccccgacgagctgtacttcgaggagggcgacatcatctacatcaccgacatgagcgacaccaactggtggaagggcaccagcaagggcagaaccggcctgatccccagcaactacgtggccgagcaggcc

SEQ ID NO: 211 - protein, 56 aa, Artificial Sequence
  Synthetic Construct
KVFRALYTFEPRTPDELYFEEGDIIYITDMSDTNWWKGTSKGRTGLIPSNYVAEQA

SEQ ID NO: 212 - protein, 34 aa, Artificial Sequence
  Cys-knots/Knottin (SOTI Var. 1)
CSPSGAICSGFGPPEQCCSAGCVLNRRARSWRCQ

SEQ ID NO: 213 - protein, 35 aa, Artificial Sequence
  Cys-knots/Knottin (SOTI-III)
CSPSGAICSGFGPPEQCCSAGACVPHPILRIFVCQ

SEQ ID NO: 214 - protein, 29 aa, Artificial Sequence
  Kalata B1
GLPVCGETCVGGTCNTPGCTCSWPVCTRN

SEQ ID NO: 215 - protein, 29 aa, Artificial Sequence
  Kalata B1
GLPVCGETCVGGTCNTPGCTCSWPVCTRN

SEQ ID NO: 216 - protein, 29 aa, Artificial Sequence
  Kalata B2
GLPVCGETCFGGTCNTPGCSCTWPICTRD

SEQ ID NO: 217 - protein, 34 aa, Artificial Sequence
  MCoTI-I
GGVCPKILQRCRRDSDCPGACICRGNGYCGSGSD

SEQ ID NO: 218 - protein, 34 aa, Artificial Sequence
  MCoTI-II
GGVCPKILKKCRRDSDCPGACICRGNGYCGSGSD

SEQ ID NO: 219 - DNA, 12 bp, Artificial Sequence
  Linker
  CDS (1)..(12)
ggcggcggcggc

SEQ ID NO: 220 - protein, 4 aa, Artificial Sequence
  Synthetic Construct
GGGG

SEQ ID NO: 221 - DNA, 12 bp, Artificial Sequence
  Linker
  CDS (1)..(12)
ggcggcggcagc

SEQ ID NO: 222 - protein, 4 aa, Artificial Sequence
  Synthetic Construct
GGGS

SEQ ID NO: 223 - DNA, 24 bp, Artificial Sequence
  Linker
  CDS (1)..(24)
ggcggcggcagcggcggcggcaga

SEQ ID NO: 224 - protein, 8 aa, Artificial Sequence
  Synthetic Construct
GGGSGGGR

SEQ ID NO: 225 - DNA, 36 bp, Artificial Sequence
  Linker
  CDS (1)..(36)
ggcggcggcagcggcggcggcagcggcggcggcaga

SEQ ID NO: 226 - protein, 12 aa, Artificial Sequence
  Synthetic Construct
GGGSGGGSGGGR

SEQ ID NO: 227 - DNA, 48 bp, Artificial Sequence
  Linker
  CDS (1)..(48)
ggcggcggcagcggcggcggcagcggcggcggcagaggcggcggcaga

SEQ ID NO: 228 - protein, 16 aa, Artificial Sequence
  Synthetic Construct
GGGSGGGSGGGRGGGR

SEQ ID NO: 229 - DNA, 60 bp, Artificial Sequence
  Linker
  CDS (1)..(60)
ggcggcggcagcggcggcggcagcggcggcggcagaggcggcggcagaggcggcggcaga

SEQ ID NO: 230 - protein, 20 aa, Artificial Sequence
  Synthetic Construct
GGGSGGGSGGGRGGGRGGGR

SEQ ID NO: 231 - DNA, 15 bp, Artificial Sequence
  Linker
  CDS (1)..(15)
ggcggcggcggcagc

SEQ ID NO: 232 - protein, 5 aa, Artificial Sequence
  Synthetic Construct
GGGGS

SEQ ID NO: 233 - DNA, 30 bp, Artificial Sequence
  Linker
  CDS (1)..(30)
ggcggcggcggcagcggcggcggcggcagc

SEQ ID NO: 234 - protein, 10 aa, Artificial Sequence
  Synthetic Construct
GGGGSGGGGS

SEQ ID NO: 235 - DNA, 45 bp, Artificial Sequence
  Linker
  CDS (1)..(45)
ggcggcggcggcagcggcggcggcggcagcggcggcggcggcagc

SEQ ID NO: 236 - protein, 15 aa, Artificial Sequence
  Synthetic Construct
GGGGSGGGGSGGGGS

SEQ ID NO: 237 - DNA, 36 bp, Artificial Sequence
  Linker
  CDS (1)..(36)
ggcggcggcagcggcggcggcggcagcggcggcagc

SEQ ID NO: 238 - protein, 12 aa, Artificial Sequence
  Synthetic Construct
GGGSGGGGSGGS

SEQ ID NO: 239 - DNA, 12 bp, Artificial Sequence
  Linker
  CDS (1)..(12)
ggcggcagcggc

SEQ ID NO: 240 - protein, 4 aa, Artificial Sequence
  Synthetic Construct
GGSG

SEQ ID NO: 241 - DNA, 24 bp, Artificial Sequence
  Linker
  CDS (1)..(24)
ggcggcagcggcggcggcagcggc

SEQ ID NO: 242 - protein, 8 aa, Artificial Sequence
  Synthetic Construct
GGSGGGSG

SEQ ID NO: 243 - DNA, 36 bp, Artificial Sequence
  Linker
  CDS (1)..(36)
ggcggcagcggcggcggcagcggcggcggcagcggc

SEQ ID NO: 244 - protein, 12 aa, Artificial Sequence
  Synthetic Construct
GGSGGGSGGGSG

SEQ ID NO: 245 - DNA, 21 bp, Artificial Sequence
  Linker
  CDS (1)..(21)
agcggcggcggcggcatcggc

SEQ ID NO: 246 - protein, 7 aa, Artificial Sequence
  Synthetic Construct
SGGGGIG

SEQ ID NO: 247 - DNA, 36 bp, Artificial Sequence
  Linker
  CDS (1)..(36)
agcggcggcggcggcagcggcggcggcggcatcggc

SEQ ID NO: 248 - protein, 12 aa, Artificial Sequence
  Synthetic Construct
SGGGGSGGGGIG

SEQ ID NO: 249 - DNA, 15 bp, Artificial Sequence
  Linker
  CDS (1)..(15)
agcggcggcggcggc

SEQ ID NO: 250 - protein, 5 aa, Artificial Sequence
  Synthetic Construct
SGGGG

SEQ ID NO: 251 - DNA, 681 bp, Homo Sapiens
  CDS (1)..(681)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 252 - protein, 227 aa, Homo Sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - DNA, 669 bp, Homo sapiens
  CDS (1)..(669)
gtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 254 - protein, 223 aa, Homo sapiens
VECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 255 - DNA, 681 bp, Homo sapiens
  CDS (1)..(681)
gacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 256 - protein, 227 aa, Homo sapiens
DTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 257 - DNA, 672 bp, Homo sapiens
  CDS (1)..(672)
cccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 258 - protein, 224 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 259 - DNA, 387 bp, Artificial Sequence
  Human IgM C-mu-4tp
  CDS (1)..(387)
aagcacccccccgccgtgtacctgctgccccccgccagagagcagctgaacctgagagagagcgccaccgtgacctgcctggtgaagggcttcagccccgccgacatcagcgtgcagtggctgcagagaggccagctgctgccccaggagaagtacgtgaccagcgcccccatgcccgagcccggcgcccccggcttctacttcacccacagcatcctgaccgtgaccgaggaggagtggaacagcggcgagacctacacctgcgtggtgggccacgaggccctgccccacctggtgaccgagagaaccgtggacaagagcaccggcaagcccaccctgtacaacgtgagcctgatcatgagcgacaccggcggcacctgctactga

SEQ ID NO: 260 - protein, 128 aa, Artificial Sequence
  Synthetic Construct
KHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 261 - DNA, 390 bp, Artificial Sequence
  Human IgA C-alpha-3tp
  CDS (1)..(390)
accttccccccccaggtgcacctgctgcccccccccagcgaggagctggccctgaacgagctgctgagcctgacctgcctggtgagagccttcaaccccaaggaggtgctggtgagatggctgcacggcaacgaggagctgagccccgagagctacctggtgttcgagcccctgaaggagcccggcgagggcgccaccacctacctggtgaccagcgtgctgagagtgagcgccgagacctggaagcagggcgaccagtacagctgcatggtgggccacgaggccctgcccatgaacttcacccagaagaccatcgacagactgagcggcaagcccaccaacgtgagcgtgagcgtgatcatgagcgagggcgacggcatctgctactga

SEQ ID NO: 262 - protein, 129 aa, Artificial Sequence
  Synthetic Construct
TFPPQVHLLPPPSEELALNELLSLTCLVRAFNPKEVLVRWLHGNEELSPESYLVFEPLKEPGEGATTYLVTSVLRVSAETWKQGDQYSCMVGHEALPMNFTQKTIDRLSGKPTNVSVSVIMSEGDGICY

SEQ ID NO: 263 - DNA, 60 bp, Artificial Sequence
  Dimerization Motif
  CDS (1)..(60)
gtggccgacttcctgatcatctacatcgaggaggcccacgccaccgacggctgggccctg

SEQ ID NO: 264 - protein, 20 aa, Artificial Sequence
  Synthetic Construct
VADFLIIYIEEAHATDGWAL

SEQ ID NO: 265 - DNA, 84 bp, Artificial Sequence
  Trimerization Motif GCN4
  CDS (1)..(84)
atcaagcagatcgaggacaagatcgaggagatcctgagcaagatctaccacatcgagaacgagatcgccagaatcaagaagctg

SEQ ID NO: 266 - protein, 28 aa, Artificial Sequence
  Synthetic Construct
IKQIEDKIEEILSKIYHIENEIARIKKL

SEQ ID NO: 267 - DNA, 117 bp, Artificial Sequence
  Trimerization Motif Matrilin 1
  CDS (1)..(117)
tgcgcctgcgagagcctggtgaagttccaggccaaggtggagggcctgctgcaggccctgaccagaaagctggaggccgtgagcaagagactggccatcctggagaacaccgtggtg

SEQ ID NO: 268 - protein, 39 aa, Artificial Sequence
  Synthetic Construct
CACESLVKFQAKVEGLLQALTRKLEAVSKRLAILENTVV

SEQ ID NO: 269 - DNA, 96 bp, Artificial Sequence
  Trimerization Motif Coronin 1a
  CDS (1)..(96)
gtgagcagactggaggaggagatgagaaagctgcaggccaccgtgcaggagctgcagaagagactggacagactggaggagaccgtgcaggccaag

SEQ ID NO: 270 - protein, 32 aa, Artificial Sequence
  Synthetic Construct
VSRLEEEMRKLQATVQELQKRLDRLEETVQAK

SEQ ID NO: 271 - DNA, 108 bp, Artificial Sequence
  Trimerization Motif CMP
  CDS (1)..(108)
gagagcctggtgaagttccaggccaaggtggagggcctgctgcaggccctgaccagaaagctggaggccgtgagcaagagactggccatcctggagaacaccgtggtg

SEQ ID NO: 272 - protein, 36 aa, Artificial Sequence
  Synthetic Construct
ESLVKFQAKVEGLLQALTRKLEAVSKRLAILENTVV

SEQ ID NO: 273 - DNA, 210 bp, Artificial Sequence
  Trimerization Motif DMPK
  CDS (1)..(210)
gaggccgaggccgaggtgaccctgagagagctgcaggaggccctggaggaggaggtgctgaccagacagagcctgagcagagagatggaggccatcagaaccgacaaccagaacttcgccagccagctgagagaggccgaggccagaaacagagacctggaggcccacgtgagacagctgcaggagagaatggagctgctgcaggccgag

SEQ ID NO: 274 - protein, 70 aa, Artificial Sequence
  Synthetic Construct
EAEAEVTLRELQEALEEEVLTRQSLSREMEAIRTDNQNFASQLREAEARNRDLEAHVRQLQERMELLQAE

SEQ ID NO: 275 - DNA, 99 bp, Artificial Sequence
  Trimerization Motif Langerin
  CDS (1)..(99)
gccagcgccctgaacaccaagatcagagccctgcagggcagcctggagaacatgagcaagctgctgaagagacagaacgacatcctgcaggtggtgagc

SEQ ID NO: 276 - protein, 33 aa, Artificial Sequence
  Synthetic Construct
ASALNTKIRALQGSLENMSKLLKRQNDILQVVS

SEQ ID NO: 277 - DNA, 87 bp, Artificial Sequence
  Trimerization Motif Surfectin Protein SP-D
  CDS (1)..(87)
gacgtggccagcctgagacagcaggtggaggccctgcagggccaggtgcagcacctgcaggccgccttcagccagtacaagaaggtg

SEQ ID NO: 278 - protein, 29 aa, Artificial Sequence
  Synthetic Construct
DVASLRQQVEALQGQVQHLQAAFSQYKKV

SEQ ID NO: 279 - DNA, 90 bp, Artificial Sequence
  Trimerization Motif Tenascin-C
  CDS (1)..(90)
gcctgcggctgcgccgccgcccccgacgtgaaggagctgctgagcagactggaggagctggagaacctggtgagcagcctgagagagcag

SEQ ID NO: 280 - protein, 30 aa, Artificial Sequence
  Synthetic Construct
ACGCAAAPDVKELLSRLEELENLVSSLREQ

SEQ ID NO: 281 - DNA, 93 bp, Artificial Sequence
  Trimerization Motif Tenascin-R
  CDS (1)..(93)
gcctgcccctgcgccagcagcgcccaggtgctgcaggagctgctgagcagaatcgagatgctggagagagaggtgagcgtgctgagagaccag

SEQ ID NO: 282 - protein, 31 aa, Artificial Sequence
  Synthetic Construct
ACPCASSAQVLQELLSRIEMLEREVSVLRDQ

SEQ ID NO: 283 - DNA, 111 bp, Artificial Sequence
  Trimerization Motif Tenascin-X
  CDS (1)..(111)
ggctgcggctgcccccccggcaccgagccccccgtgctggccagcgaggtgcaggccctgagagtgagactggagatcctggaggagctggtgaagggcctgaaggagcag

SEQ ID NO: 284 - protein, 37 aa, Artificial Sequence
  Synthetic Construct
GCGCPPGTEPPVLASEVQALRVRLEILEELVKGLKEQ

SEQ ID NO: 285 - DNA, 108 bp, Artificial Sequence
  Tetrameric Motif CMP (R27Q)
  CDS (1)..(108)
gagagcctggtgaagttccaggccaaggtggagggcctgctgcaggccctgaccagaaagctggaggccgtgagcaagcagctggccatcctggagaacaccgtggtg

SEQ ID NO: 286 - protein, 36 aa, Artificial Sequence
  Synthetic Construct
ESLVKFQAKVEGLLQALTRKLEAVSKQLAILENTVV

SEQ ID NO: 287 - DNA, 135 bp, Artificial Sequence
  Pentameric Motif (COMP)
  CDS (1)..(135)
gacctggccccccagatgctgagagagctgcaggagaccaacgccgccctgcaggacgtgagagagctgctgagacagcaggtgaaggagatcaccttcctgaagaacaccgtgatggagtgcgacgcctgcggc

SEQ ID NO: 288 - protein, 45 aa, Artificial Sequence
  Synthetic Construct
DLAPQMLRELQETNAALQDVRELLRQQVKEITFLKNTVMECDACG

SEQ ID NO: 289 - protein, 20 aa, Artificial Sequence
  Signal Peptide ASP1
MWWRLWWLLLLLLLLWPMVA

SEQ ID NO: 290 - protein, 21 aa, Artificial Sequence
  Signal Peptide ASP2
MRPTWAWWLFLVLLLALWAPG

SEQ ID NO: 291 - protein, 21 aa, Artificial Sequence
  Signal Peptide ASP3
MKVQWLLLWVLLLLVLFCSRG

SEQ ID NO: 292 - protein, 20 aa, Artificial Sequence
  Signal Peptide ASP4
MRPWTWVLLLLLLICAPSYA

SEQ ID NO: 293 - protein, 19 aa, Artificial Sequence
  Signal Peptide ASP5
MMWLWLVLLLLCLAGNVQA

SEQ ID NO: 294 - protein, 22 aa, Artificial Sequence
  Signal Peptide ASP6
MPPKKCLLLLLTLLLLISTTFG

SEQ ID NO: 295 - protein, 19 aa, Artificial Sequence
  Signal Peptide ASP7
MAGGVAGLLLALLLPSALS

SEQ ID NO: 296 - protein, 19 aa, Artificial Sequence
  Signal Peptide ASP8
MKLLLIFFVLVVWMGPAHR

SEQ ID NO: 297 - protein, 20 aa, Artificial Sequence
  Signal Peptide ASP 9
MVRGVLALLLMALQMDASSG

SEQ ID NO: 298 - protein, 20 aa, Artificial Sequence
  Signal Peptide ASP10
MSADCSWGAAFGALLPLAAG

SEQ ID NO: 299 - protein, 19 aa, Artificial Sequence
  Signal Peptide ASP11
MTKHLGVLFAGFTSADVSA

SEQ ID NO: 300 - protein, 19 aa, Artificial Sequence
  Signal Peptide ASP12
MIFNPMVVFLFCVSNHALR

SEQ ID NO: 301 - protein, 20 aa, Artificial Sequence
  Signal Peptide ASP13
MDLVSWTFMEVSTLVLPKRP

SEQ ID NO: 302 - protein, 25 aa, Artificial Sequence
  Signal Peptide ASP14
MLAALRRACTSACRVPIKPTHLAQG

SEQ ID NO: 303 - DNA, 18 bp, Artificial Sequence
  GFP Forward Primer
gaagttcgagggcgacac

SEQ ID NO: 304 - DNA, 30 bp, Artificial Sequence; identical to SEQ ID NO: 4
  GFP Reverse Primer

(19) United States
(12) Patent Application Publication
(10) Pub. No.: US 2022/0002752 A1
(43) Pub. Date: Jan. 6, 2022

(54) RECOMBINANT VECTORS COMPRISING GENES FOR BINDING DOMAINS AND SECRETABLE PEPTIDES

(71) Applicant: DENOVO BIOPHARMA LLC, San Diego, CA (US)

(72) Inventors: Amy H. LIN, San Diego, CA (US); Douglas J. JOLLY, Encinitas, CA (US)

(73) Assignee: DENOVO BIOPHARMA LLC, San Diego, CA (US)

(21) Appl. No.: 17/292,357

(22) PCT Filed: Nov. 13, 2019
(86) PCT No.: PCT/US19/61297

§ 371 Date: May 7, 2021

Related U.S. Application Data
(60) Provisional application No. 62/760,912, filed on Nov. 13, 2018.
(60) Provisional application No. 62/893,673, filed on Aug. 29, 2019.

(51) Int. Cl. C12N 15/86; C12N 7/00

(52) U.S. Cl. CPC C12N 15/86; C12N 7/00; C12N 2770/36121; C12N 2770/36122; C12N 2770/36143

(57) ABSTRACT

This disclosure provides modified recombinant retroviruses comprisings a transgene encoding a protein with a heterologous secretion signal, containing a 2A-peptide or peptide-like coding sequence operably linked to a heterologous polynucleotide, The disclosure further relates to cells and vector expressing or comprising such vectors and methods of using such modified vectors in the treatment of disease and disorders.

### CROSS REFERENCE TO RELATED APPLICATIONS

This application claims priority to U.S. Provisional Application Ser. No. 62/760,912, filed Nov. 13, 2018 and U.S. Provisional Application Ser. No. 62/893,673, filed Aug. 29, 2019, the disclosures of which are incorporated herein by reference.

### REFERENCE TO SEQUENCE LISTING

The present application is being filed along with a Sequence Listing in electronic format. The Sequence Listing is provided as a file entitled Sequence-Listing_ST25.txt, created Nov. 13, 2019, which is 408,816 bytes in size. The information in the electronic format of the Sequence Listing is incorporated herein by reference in its entirety.

### TECHNICAL FIELD

This disclosure relates to viral vectors. The disclosure further relates to the use of such viral vectors for delivery and expression of heterologous nucleic acids in cells and their expression and secretion.

### BACKGROUND

Effective methods of delivering genes and heterologous nucleic acids to cells and subjects has been a goal of researchers for scientific development and for possible treatments of diseases and disorders.

### SUMMARY

The disclosure provides viruses comprising a 2A-peptide cassette containing a secretory peptide coding sequence downstream of the 2A-peptide and upstream of a heterologous gene to be secreted. Further embodiments comprise heterologous genes which encode antibodies, single-chain antibodies, or other antibody related structures, binding proteins that are derived from non-immunoglobulin scaffold proteins and the like. In a further embodiment the antibody-related peptides or non-immunoglobulin binding proteins comprise sequences that lead to multimerization of the binding proteins to provide higher binding affinity for the target entity. Yet further embodiments comprise viruses that comprise heterologous genes with a heterologous secretion signal, to both virus and gene, upstream of a heterologous gene product to be secreted.

This disclosure further describes polypeptide subunits of both immunoglobulin (Ig) and non-immunoglobulin (non-Ig) scaffold proteins, each including a fusion polypeptide of the antigen-binding domain, a multimerization domain, e.g., dimerization, trimerization and pentameric domain, and, optionally, an IgG Fc domain, which are capable of forming stable homo- and dimeric proteins. Oligomeric complexes of the non-Ig scaffold proteins can also be formed by single or multiple Gly-Ser linkers.

The disclosure includes engineered Ig scaffold protein which include heavy chain variable domains derived from human, mouse, camel (camelid), shark and cow (Curr Opin Struct Biol. 2017 August; 45:10-16. doi: 10.1016/j.sbi.2016.10.019, incorporated herein by reference), (Nat Biotechnol. 2017 Dec. 8; 35(12):1115-1117. doi: 10.1038/nbt1217-1115, incorporated herein by reference) as well as non-Ig scaffold proteins (see, for example: Skrlec et al., Trends Biotechnol., 33(7):408-18, July 2015; and Simeon & Chen Protein& Cell 9:2-14, 2018; both of which are incorporated herein by reference) which include Adnectins, Affibodies, Affilins, Affimers, Anticalins, Atrimers, Avimers, Centryrins, DARPins, Bynomers, Cys-knots, Kunitz domains, OBodies, Pronectins, Tn3s, Hcks, NPHP1s, Tecs, Amphs, RIMBP #3, IRIKS, SNX33, Eps8L1, FISH #5, CMS #1, and OSTF1, all of which can be operably linked to the N-terminus portion of the Fc of human IgG which allows dimerization of monomeric or oligomeric scaffold protein via disulfide bond formation to form a highly complex oligomeric proteins.

Compositions and methods are provided that are useful for cancer immunotherapy delivered by viral vectors, including retroviral replicating vector and retroviral non-replicating vectors, other viral vectors, oncolytic viral vectors and non-viral expression vectors.

In one embodiment the non-Ig scaffold is of human origin to minimize anti-scaffold protein immune responses.

In one embodiment, the antigen-specific binding subunit of non-Ig scaffold proteins function as agonists or antagonist targeting CTLA-4, PD-1, PDL1, GITR, ICOS, LAG-3, TIM-3, OX40, CD40L, CD137/4-1BB, CD27, TIGIT, VISTA, BTLA, IL-2R alpha, IL-2R beta, IL-2R gamma, IL-15R alpha, IL-15R beta, or IL-15R gamma, CD19, CD20, mesothelin, ganglioside GD2, fibroblast associated protein FAP, BCMA, CD3, FOXP3, IL-12R alpha or beta, CD47, SIRP alpha, CD94/NKG2, CD244/2B4, adenosine receptor A2A, EGFR, EGF, VEGFR, VEGF, PDGFR, PDGF, HGFR/MET,HGF, IGF-IR, IGF-1, HER-1, HER-2, HER-3, CEA, EB-D, TRAILR1/DR4, TRAILR2/DR5, Extradomain B (ED-B), IL-10 and IL-35.

In another embodiment, the antigen-specific binding subunit of non-Ig scaffold proteins function as agonists or antagonist targeting at least one of interleukins 1 through 38 which consists of greater than 60 current members; and their receptors such as IL-10 and IL-35 receptors of the IL-2 family, which is composed of IL-2, IL-4, IL-7, IL-9, IL-15, and IL-21 (these receptors contain the common cytokine receptor γ chain (CD132, γc)); IL-13R shares IL-4Ra with IL-4, receptors for IL-4 and IL-13 consist of 2 receptor chains—IL-4 and IL-13 bind to IL-4R, which consists of IL-4Ra (CD124) and the IL-13Rα1 chain and IL-13R consists of 2 subunits, IL-13Rα1 and IL-13Ra2, and signaling occurs through the IL-4R complex type II, which consists of IL-4Ra and IL-13Ra; TSLPR (CRFlR-2) shares IL-7R with IL-7; receptors for IL-3, IL-5, and GM-CSF which are heterodimers with a unique a chain and the common s chain (sc, CD131) subunit; IL-10 family members (IL-10, IL-19, IL-20, IL-22, IL-24, IL-26, IL-28, and IL-29) and the corresponding receptors which share common receptor subunits, as shown; TNF-α and its receptors TNFRI and TNFR2; TGF-β and its heterodimer receptor consisting of TGF-βR1 and TGF-βR2; IL12 and its receptor IL-12R consisting of 2 subunits: IL-12Rβ1 and IL-12Rβ3. IL-23 and/or its heterodimer receptor subunits, IL-12Rβ1 and IL-23R; IFN-α and IFN-β and/or their heterodimer receptor consisting of IFNAR1 and IFNAR2; IFN-γ and/or its heterodimeric receptor subunits IFN-γR1 and IFN-γR2.

In one embodiment, the antigen binding domain of non-Ig scaffold proteins are fusion proteins each includes antigen-binding non-Ig scaffold proteins, glycine-serine linkers, functional multimerization domain wherein the non-Ig scaffold proteins can self-assemble into a homodimeric, homotrimeric, homopentameric protein complex, homohexameric or other types of protein complexes, including heteromeric complexes.

In one embodiment, homohexameric non-Ig scaffold protein complex are fusion proteins that include 6 antigen-binding non-Ig scaffold proteins each consisting a non-Ig scaffold protein, glycine-serine linkers, a functional trimerization domain, and an IgG Fc domain.

In one embodiment, homodecameric non-Ig scaffold protein complex are fusion proteins that include 10 antigen-binding non-Ig scaffold proteins each consisting a non-Ig scaffold protein, glycine-serine linkers, a functional pentameric domain, and an IgG Fc domain.

In another embodiment, the antigen-binding domain of non-Ig scaffold proteins are multivalent fusion protein complex that include different antigen-binding non-Ig scaffold proteins, glycine-serine linkers wherein the non-Ig scaffold proteins can self-assemble into a hetero-dimeric, hetero-trimeric, or hetero-multimeric proteins.

In one embodiment, methods to promote survival or proliferation of antigen-experienced T cells and/or activated NK cells and dendritic cells for neoantigen priming wherein the non-Ig scaffold protein in oligomeric form can specifically bind to antigen on the surface of the tumor cells, T cells, NK cells, dendritic cells, myeloid cells, tumor associated fibroblasts, B cells.

In a further embodiment the transgene encodes a prodrug activating protein which prodrug activating protein has been made as secretable peptide or protein. In a further embodiment the prodrug activating transgene is a yeast derived cytosine deaminase.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 shows a sequence alignment of amino acid sequence of the 2A regions of foot-and-mouth disease virus (F2A), equine rhinitis A virus (E2A), Thosea asigna virus (T2A) and porcine teschovirus-1 (P2A) (SEQ ID Nos: 55 to 58).

FIG. 2 shows a sequence alignment of 2A peptide sequences present in different classes of viruses (SEQ ID Nos: 59 to 125).

FIG. 3 is a schematic diagram of RRV-scFv-PDL1 plasmid DNAs. (A) Two pairs of single-chain variable fragment (scFv) against PD-L1 were encoded in pAC3 RRV backbone. One pair consists of scFv with and without the Fc from human IgG1, designated as pAC3-scFv-PDL1 and pAC3-scFvFc-PDL1, respectively. Another pair consists of scFv-PDL1 and scFvFc-PDL1 with HA and Flag epitope incorporated at the C-terminus, designated as pAC3-scFv-HF-PDL1, pAC3-scFvFc-HF-PDL1. Filled grey rectangle indicates 2A peptide, IRES or a mini-promoter placed downstream of the env gene; filled black rectangle (SP=signal peptide, Table A) indicates secretion/leader sequence, for example, derived from human IL-2.

FIG. 4A-B shows PDL1scFv and PDL1scFvFc protein expression and the separation efficiency of Env-scFv and Env-ScFvFc polyproteins in transiently transfected 293T cells. (A) scFv-Tag (˜30 KDa) and scFvFc-Tag (˜55 Kd) protein expression from HEK293T cells transiently transfected with of pAC3-GSG-T2A-PDL1scFv, pAC3-GSG-T2A-PDL1scFvFc, pAC3-GSG-T2A-PDL1scFv-Tag, pAC3-GSG-T2A-PDL1scFvFc-Tag. (B) Anti-2A immunoblot of cell lysates from transiently transfected 293T cells. The protein band detected above ˜110 KDa represents the Env-scFv and Env-ScFvFc fusion polyproteins. The protein band detected at ˜85 KDa represents the Pr85 viral envelope protein separated from the fusion polyprotein, and protein band detected at ˜15 KDa represents the p15E-2A protein processed from the Pr85 viral envelope protein.

FIG. 5 shows Western blot analysis of viral envelope proteins produced transient transfection in 293T cells. Twenty micrograms of total protein lysates were loaded per well. Membranes were incubated with (left panel) anti-HA which detects HA- and Flag-tagged scFv-PD-L1 and scFvFc-PD-L1 or (right panel) anti-2A peptide antibody which detects Env-scFv polyprotein (Env-scFv), unprocessed viral precursor envelop protein separated from the Env-scFv polyprotein (Env-2A), and processed viral envelop protein tagged with the 2A peptide at the C-terminus (p15E-2A). Anti-GAPDH antibody (lower left panel) which the house keeping protein GAPDH was included as loading control.

FIG. 6A-B show detection of scFv PD-L1 binding to PD-L1 by competitive ELISA. Wells in a 96-well microtiter plate were coated with (A) recombinant human or (B) mouse PD-L1-Fc followed by co-incubation with His-tagged recombinant PD-1-Fc in competition with supernatant of undefined scFv PD-L1 (scFv) and scFvFc PD-L1 (scFvFc) protein concentration collected from CT26 cells maximally infected with RRV-scFv-PDL1 and RRV-scFvFc-PDL1, respectively. Anti-PD-L1 antibody was included as positive control. Anti-6× His tag antibody was used to detect bound His-tagged PD-1-Fc. Optical density was measured at 450 nm. The percentage of inhibition was calculated with respect to the supernatant from CT26 maximally infected with RRV-GFP (non-scFv-PD-L1) used in the competition. Error bars indicate the standard deviation of the dataset.

FIG. 7A-B shows scFv PD-L1 trans-binding activity to PD-L1 on the cell surface of bystander cells. IFNγ-treated EMT6 cells maximally infected with RRV-scFv-HF PDL1 (HA-tagged scFv-PD-L1) or RRV-GFP at indicated ratios were split into 2 sets. (A) One set of cells was stained with Alexa Fluor 647-conjugated anti-HA antibody and (B) the second set of cells was stained with PE-conjugated anti-mouse PD-L1 antibody. HA-positive, PD-L1-positive, and GFP-positive cell populations were measured by flow cytometric analysis.

FIG. 8A-D shows pre-transduced tumor cells expressing scFv PD-L1 and scFvFc PD-L1 that demonstrate a dose-dependent anti-tumor activity. (A) Orthotopic breast cancer model using EMT6 tumor cells pre-transduced with RRV-scFv-PDL1 or RRV-scFvFc-PDL1 mixed with tumor cells pre-transduced with RRV-GFP at indicated ratios were implanted in the mammary fat pad in 8-week-old BALB/c female mice (n=10 per group). Survival was monitored for 90 days. Anti-PD-1 antibody was included as a control and was i.p. administered on day 10 (300 μg per mouse), day 13, day 16, and day 19 (200 μg per mouse). *p=0.2529 for 0% scFv/scFvFc vs anti-PD-1; **p=0.2529 for 0% vs 2%; ***p=0.0919 for 0% vs 30%; ****p=0.1674 for 0% vs 100%. Ticks on the graph indicate mice censored due to tumor necrotic and were terminated; these mice were not scored as death and were not excluded from the graph. (B-D) Mice that survived initial tumor implant from RRV-scFv-PDL1 and RRV-scFvFc-PDL1 treated groups (n=5) were challenged with 1×10⁶ EMT6 tumor cells on the flank and tumor growth was monitored overtime. Naïve animals (n=5) were included as controls. Error bars indicate SEM of the dataset.

FIG. 9A-B shows data from an orthotopic glioma model with intracranial injection of RRV-scFv-PDL1 that demonstrates a dose-dependent anti-tumor activity. (A) Female B6C3F1 mice (8-week-old; n=10 per group) were i.c. implanted with 1×10⁴ of Tu-2449 cells. Survival analysis was monitored for 90 days. Mice in the experimental groups were injected with purified RRV-scFv-PDL1 of 1×10⁵ or 1×10⁶ transduction unit (TU) on day 4 post tumor implant. Control groups are mice bearing 100% pre-transduced scFv-PD-L1 expressing tumor cells and mice treated anti-PD-1 antibody or isotype control. Tu-2449 cells 100% pre-transduced with RRV-scFv-PDL1 expressing scFv PD-L1 and anti-PD-1 antibody (300 μg per mouse i.p. induction on day 4; 200 μg per mouse maintenance dose on day 10, 14 and 17) were included as controls. Survival data were plotted by the Kaplan-Meier method. Statistical significance of survival between mice treated with isotype and 100% pre-transduced with RRV-scFv-PD-L1 or injection-treated RRV-scFv-PDL1 group was determined by the Log-rank (Mantel-Cox). (B) Mice which had survived from initial tumor implant from RRV-scFv-PDL1 treated groups were challenged with 2×10⁶ Tu-2449 cells on the right flank. Tumor growth and measurement were monitored over time. Error bars indicate the SEM of the dataset.

FIG. 10 shows detection of the epitope-tagged Affimer-SQT protein by direct immunoblotting and immunoprecipitation from supernatant of pAC3-gT2A-Affimer-SQT transiently transfected 293T cells.

FIG. 11A-B shows detection of the epitope-tagged Hck protein by direct immunoblotting and immunoprecipitation from supernatant of pAC3-gT2A-Hck shown in (A) and pAC3-IRES-Hck indicated by an arrow in (B) transiently transfected 293T cells.

FIG. 12 shows schematic diagrams of RRV-scaffold plasmid DNAs. Antigen binding domains derived from non-Ig scaffold [0066] is encoded in pAC3-2A, pAC3-IRES or pAC3-minipromoter backbone. Filled grey rectangle indicates 2A peptide, IRES or a mini-promoter placed downstream of the env gene to direct expression of the transgene; a filled black rectangle indicates a leader sequence (Table A). Oligomerization domain (Table 4, 5 and 6) can be placed at the N- or C-terminus of the non-Ig scaffold with linker(s) to form oligomers. Also shown in last two lines are configurations that lead to secretion of bispecifc and trispecific binding molecules with properties for linking responses against two or three targets like bispecific or tripsecific antibodies (Labrijn et al., Nature Rev. Drug Disc. 18:585-608 2019).

FIG. 13 shows a schematic diagram of RRV-syCD2 plasmid DNAs. The secreted form of yCD2 is encoded in pAC3-2A, pAC3-IRES, pAC3-minipromoter backbone. Filled grey rectangle indicates 2A peptide, IRES or a mini-promoter placed downstream of the env gene to direct expression of the transgene; a filled black rectangle indicates a signal peptide (SP), (Table A).

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the vector” includes reference to one or more vectors, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

General texts which describe molecular biological techniques useful herein, including the use of vectors, promoters and many other relevant topics, include: Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”); and S. Carson, H. B. Miller & D. S. Witherow and Molecular Biology Techniques: A Classroom Laboratory Manual, Third Edition, Elsevier, San Diego (2012). Examples of protocols sufficient to direct persons of skill through in vitro amplification methods, including the polymerase chain reaction (PCR), the ligase chain reaction (LCR), Q$-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The publications discussed throughout the text are provided solely for their disclosure prior to the filing date of the present application. Nothing herein is to be construed as an admission that the inventors are not entitled to antedate such disclosure by virtue of prior disclosure.

The terms “express” and “expression” mean allowing or causing the information in a gene or DNA sequence to become manifest, for example producing a protein by activating the cellular functions involved in transcription and translation of a corresponding gene or DNA sequence or in the case of inhibitor RNA (RNAi) transcribing the RNAi molecule such that is is processed and capable of inhibiting expression of a target gene.

A DNA sequence is expressed in or by a cell to form an “expression product” such as a polypeptide or protein. The expression product itself, e.g., the resulting polypeptide or protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

As mentioned above, in some instances the term “express” includes the production of inhibitory RNA molecules (RNAi). The expression of such molecules does not involve the translation machinery of the cell but rather utilizes machinery in a cell to modify a host cell's gene expression. In some embodiments, a recombinant viral vector of the disclosure can be modified to deliver a coding sequence (e.g., a polypeptide or protein), an RNAi molecule, or both a coding sequence (e.g., express a polypeptide or protein) and an RNAi molecule to a host cell that can then express the coding sequence and/or RNAi molecule.

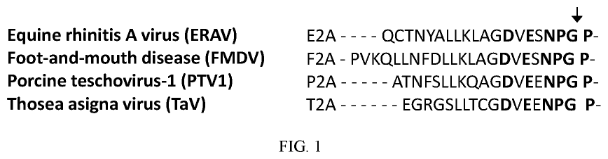

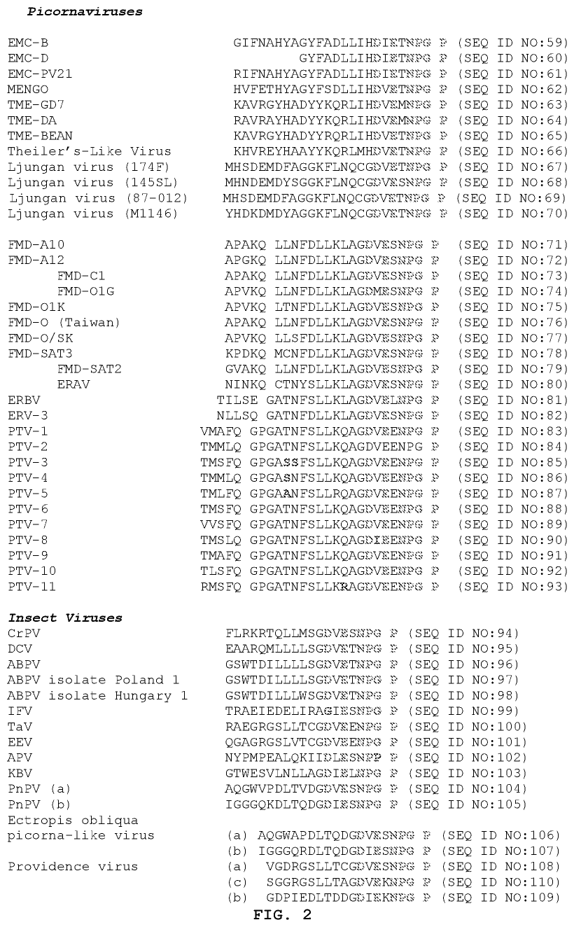

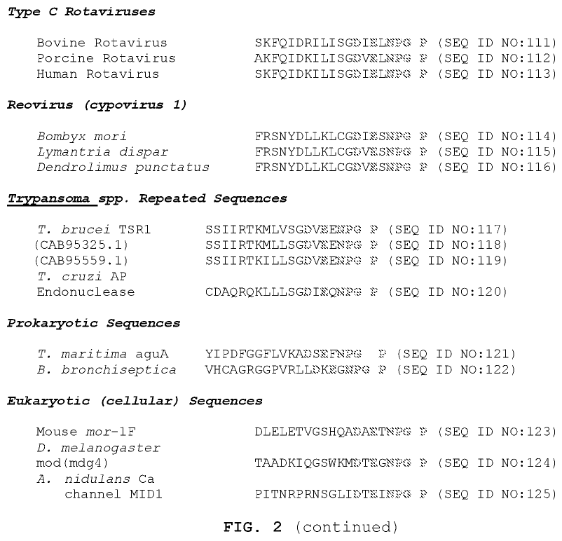

A “2A peptide or 2A peptide-like sequence” refers to a peptide having the consensus sequence of SEQ ID NO:1, a sequence that is 97% identical to any of the sequences in FIGS. 1 and 2 and which contains the consensus sequence of SEQ ID NO:1. A sequence that “encodes” a 2A peptide or 2A peptide-like sequence is a polynucleotide sequence that encodes a 2A peptide or peptide-like sequence having, e.g., the consensus sequence of SEQ ID NO:1. The coding sequence is operably linked to and placed, in one embodiment, between an ENV and heterologous sequence, such that once the sequence is transcribed it is transcribed as a single transcript (e.g., polymRNA) and when the transcript is translated that two polypeptide are produced (e.g., the ENV and the heterologous polypeptide).

An internal ribosome entry sites (“IRES”) refers to a segment of nucleic acid that promotes the entry or retention of a ribosome during translation of a coding sequence usually 3′ to the IRES. In some embodiments the IRES may comprise a splice acceptor/donor site, however, preferred IRESs lack a splice acceptor/donor site. Normally, the entry of ribosomes into messenger RNA takes place via the cap located at the 5′ end of all eukaryotic mRNAs. However, there are exceptions to this universal rule. The absence of a cap in some viral mRNAs suggests the existence of alternative structures permitting the entry of ribosomes at an internal site of these RNAs. To date, a number of these structures, designated IRES on account of their function, have been identified in the 5′ noncoding region of uncapped viral mRNAs, such as that of picornaviruses, in particular the poliomyelitis virus (Pelletier et al., 1988, Mol. Cell. Biol., 8, 1103-1112) and the EMCV virus (encephalo-myocarditis virus (Jang et al., J. Virol., 62, 2636-2643 1988; B. T. Baranick et al., Proc Natl Acad Sci USA. 105:4733-8, 2008). The disclosure provides the use of an IRES in the context of a replication-competent retroviral vector.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. The regulatory sequence may be homologous or heterologous to the desired gene sequence. For example, a wide range of promoters may be utilized, including viral or mammalian promoter.

The term “regulatory nucleic acid sequence” refers collectively to promoter sequences/regions, polyadenylation signals, transcription termination sequences, upstream regulatory domains, origins of replication, enhancers and the like, which collectively provide for the replication, transcription and translation of a coding sequence in a recipient cell. Not all of these control sequences need always be present so long as the selected coding sequence is capable of being replicated, transcribed and translated in an appropriate host cell. One skilled in the art can readily identify regulatory nucleic acid sequence from public databases and materials. Furthermore, one skilled in the art can identify a regulatory sequence that is applicable for the intended use, for example, in vivo, ex vivo, or in vitro.

As used herein, the term “RNA interference” (RNAi) refers to the process of sequence-specific post-transcriptional gene silencing mediated by short interfering nucleic acids (siRNAs or microRNAs (miRNA)). The term “agent capable of mediating RNA interference” refers to siRNAs as well as DNA and RNA vectors that encode siRNAs when transcribed within a cell. The term siRNA or miRNA is meant to encompass any nucleic acid molecule that is capable of mediating sequence specific RNA interference, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

The terms “secretory signal domain” or “secretory signal peptide” (SSP) or “signal peptide” means a short peptide typically located at the N-terminus as part of a precursor protein sequence. Translational machinery in eukaryotic cells utilizes these short peptides to sort proteins to targeted destinations. General characteristics of an SSP consist of three domains: (1) N-region: the positive-charged domain, (2) H-region: the hydrophobic core and (3) C-region: the cleavage site (Owji et al., Euro J. of Cell Biol., 2018). SSPs are cleaved off from their passenger protein or polypeptide by the endoprotease SPase I. The polypeptide or protein expression level is not only related to translational efficiency but also to translocation efficiency determined by the secretory machinery and SSPs. The sequences of SSP can influence the translocation efficiency and thus combinations of heterologous SSPs linked to the passenger polypeptide or protein can be engineered at the nucleic acid level to modulate the level of secreted proteins (Kober et al., 2013; Zamani et al., 2015; Negahdaripour et al., 2017; Mousavi et al., 2017). Further, there are artificial SSPs designed to enhance protein secretion in both prokaryotic and eukaryotic systems (Barash et al., Biochem and Biophy Res Comm., 2002; Clerico et al., Biopolymers, 2008). Although the existence and general function of SSPs has been known for decades, the ability of SSPs to enable functional non-native expressed gene products to be secreted from a host cell, especially when combined with other membrane proteins, e.g., the ENV protein of retroviral vectors and the 2A expression system, has previously not been described.

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise DNA or RNA, into which foreign DNA encoding a protein, polypeptide, nucleic acid etc. is inserted by restriction enzyme technology. A common type of vector is a “plasmid”, which generally is a self-contained molecule of double-stranded DNA that can readily accept additional (foreign) DNA and which can be readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.). Many appropriate host cells, using methods disclosed or cited herein or otherwise known to those skilled in the relevant art have been used in such transfections. Recombinant cloning vectors will often include one or more replication systems for cloning or expression, one or more markers for selection in the host, e.g., antibiotic resistance, and one or more expression cassettes.

The disclosure provides methods and compositions useful for gene or protein delivery to a cell or subject. In one such embodiment, the methods and compositions are such that the protein or polypeptide will be secreted from the cells that have taken up the gene encoding the protein or polypeptide. Such methods and compositions can be used to treat various diseases and disorders in a subject including cancer and other cell proliferative diseases and disorders. The disclosure provides replication competent viral vectors for gene delivery to a cell and in one embodiment, the viral vectors are replication competent retroviral vectors.

The disclosure provides viral vectors the contain a heterologous polynucleotide encoding, for example, a cytosine deaminase or mutant thereof, an miRNA or siRNA, a cytokine, an antigen binding domain (e.g., antibody or antibody fragment; or non-antibody binding domain), non-immunoglobulin (Ig) scaffold protein, or combinations of coding sequences etc., that can be delivered to a cell or subject. The viral vector can be an adenoviral vector, a measles vector, a herpes vector, a retroviral vector (including Alpha-, Beta-, Gamma-, Delta-retroviral vector, Spumavirus vector such as Simian Foamy Virus (SFV) or Human Foamy Virus (HFV), or lentiviral vector), a rhabdoviral vector such as a Vesicular Stomatitis viral vector, a reovirus vector, a Seneca Valley Virus vector, a poxvirus vector (including animal pox or vaccinia derived vectors), a parvovirus vector (including an AAV vector), an alphavirus vector or other viral vector known to one skilled in the art (see also, e.g., Concepts in Genetic Medicine, ed. Boro Dropulic and Barrie Carter, Wiley, 2008, Hoboken, N.J.; The Development of Human Gene Therapy, ed. Theodore Friedmann, Cold Springs Harbor Laboratory Press, Cold springs Harbor, New York, 1999; Gene and Cell Therapy, ed. Nancy Smyth Templeton, Marcel Dekker Inc., New York, N.Y., 2000 and Gene & Cell Therapy: Therapeutic Mechanism and Strategies, 3^{rd}. ed., ed. Nancy Smyth Templetone, CRC Press, Boca Raton, Fla., 2008; the disclosures of which are incorporated herein by reference).

As described below, the RRVs of the disclosure can be derived from (i.e., the parental nucleotide sequence is obtained from) MLV, MoMLV, GALV, FELV and the like and are engineered to contain a 2A peptide or 2A like-peptide operably linked to a heterologous nucleotide sequence (sometimes referred to herein as a “2A-peptide cassette”). In some instances the 2A peptide or 2A like-peptide is separated from the heterologous nucleotide sequence by an oligonucleotide encoding a secretory signal peptide.

A recombinant replication competent retroviral vector or retroviral replicating vector (RRV) refers to a vector based on a member of the retroviridae family of viruses. The structures of retroviruses are well characterized as described more fully below. Retroviruses have been classified in various ways, but the nomenclature has been standardized in the last decade (see ICTVdB—The Universal Virus Database, v 4 on the World Wide Web (www) at ncbi.nlm.nih.gov/ICTVdb/ICTVdB/and the text book “Retroviruses” Eds. Coffin, Hughs and Varmus, Cold Spring Harbor Press 1997; the disclosures of which are incorporated herein by reference). Such vectors can be engineered using recombinant genetic techniques to modify the parent virus to be a non-naturally occurring RRV by inserting heterologous genes or sequences. Such modification can provide attributes to the vectors that allow them to deliver genes to be express to a host cell in vitro or in vivo.

Retroviruses are defined by the way in which they replicate their genetic material. During replication the RNA genome of the virus is converted into DNA (termed proviral DNA). Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virion.

The retrovirus particle is composed of two identical RNA molecules. Each wild-type genome has a positive sense, single-stranded RNA molecule, which is capped at the 5′ end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer (lipid envelop), derived from host cell membranes and containing viral envelope (env) proteins. The env proteins bind to a cellular receptor for the virus and the particle typically enters the host cell via receptor-mediated endocytosis and/or membrane fusion.

After release of the viral particle into a targeted cell, the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome.

The double-stranded linear DNA produced by reverse transcription may, or may not, have to be circularized in the nucleus. The provirus now has two identical repeats at either end, known as the long terminal repeats (LTR). The termini of the two LTR sequences produces the site recognized by a pol product—the integrase protein—which catalyzes integration, such that the provirus is always joined to host DNA two base pairs (bp) from the ends of the LTRs. A duplication of cellular sequences is seen at the ends of both LTRs, reminiscent of the integration pattern of transposable genetic elements. Retroviruses can integrate their DNAs at many sites in host DNA, but different retroviruses have different integration site preferences. HIV-1 and simian immunodeficiency virus DNAs preferentially integrate into expressed genes, murine leukemia virus (MLV) DNA preferentially integrates near transcriptional start sites (TSSs), and avian sarcoma leukosis virus (ASLV) and human T cell leukemia virus (HTLV) DNAs integrate nearly randomly, showing a slight preference for genes (Derse D, et al. (2007), J Virol 81:6731-6741; Lewinski M K, et al. (2006), PLoS Pathog 2:e601).

Transcription, RNA splicing and translation of the integrated viral DNA is mediated by host cell proteins. Variously spliced transcripts are generated. In the case of the human retroviruses HIV-1/2 and HTLV-I/II viral proteins are also used to regulate gene expression. The interplay between cellular and viral factors is a factor in the control of virus latency and the temporal sequence in which viral genes are expressed.

Retroviruses can be transmitted horizontally and vertically. Efficient infectious transmission of retroviruses requires the expression on the target cell of receptors which specifically recognize the viral envelope proteins, although viruses may use receptor-independent, nonspecific routes of entry at low efficiency. Normally a viral infection leads to a single or few copies of viral genome per cell because of receptor masking or down-regulation that in turn leads to resistance to superinfection (Ch3 p104 in “Retroviruses”, J M Coffin, S H Hughes, & H E Varmus, 1997, Cold Spring Harbor Laboratory Press, Cold Spring Harbor N.Y.; Fan et al. J. Virol 28:802, 1978). By manipulating the situation in tissue culture it is possible to get some level of multiple infection but this is typically less than 5 copies/diploid genome. In addition, the target cell type must be able to support all stages of the replication cycle after virus has bound and penetrated. Vertical transmission occurs when the viral genome becomes integrated in the germ line of the host. The provirus will then be passed from generation to generation as though it were a cellular gene. Hence endogenous proviruses become established which frequently lie latent, but which can become activated when the host is exposed to appropriate agents.

The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2) and simian immunodeficiency virus (SIV).

The oncoviruses have historically been further subdivided into groups A, B, C and D on the basis of particle morphology, as seen under the electron microscope during viral maturation. A-type particles represent the immature particles of the B- and D-type viruses seen in the cytoplasm of infected cells. These particles are not infectious. B-type particles bud as mature virion from the plasma membrane by the enveloping of intracytoplasmic A-type particles. At the membrane they possess a toroidal core of 75 nm, from which long glycoprotein spikes project. After budding, B-type particles contain an eccentrically located, electron-dense core. The prototype B-type virus is mouse mammary tumor virus (MMTV). No intracytoplasmic particles can be observed in cells infected by C-type viruses. Instead, mature particles bud directly from the cell surface via a crescent ‘C’-shaped condensation which then closes on itself and is enclosed by the plasma membrane. Envelope glycoprotein spikes may be visible, along with a uniformly electron-dense core. Budding may occur from the surface plasma membrane or directly into intracellular vacuoles. The C-type viruses are the most commonly studied and include many of the avian and murine leukemia viruses (MLV). Bovine leukemia virus (BLV), and the human T-cell leukemia viruses types I and II (HTLV-I/II) are similarly classified as C-type particles because of the morphology of their budding from the cell surface. However, they also have a regular hexagonal morphology and more complex genome structures than the prototypic C-type viruses such as the murine leukemia viruses (MLV). D-type particles resemble B-type particles in that they show as ring-like structures in the infected cell cytoplasm, which bud from the cell surface, but the virion incorporate short surface glycoprotein spikes. The electron-dense cores are also eccentrically located within the particles. Mason Pfizer monkey virus (MPMV) is the prototype D-type virus.

In many situations for using a recombinant replication competent retrovirus therapeutically, it is advantageous to have high levels of expression of the transgene that is encoded by the recombinant replication competent retrovirus. For example, with a prodrug activating gene such as the cytosine deaminase gene it is advantageous to have higher levels of expression of the CD protein in a cell so that the conversion of the prodrug 5-FC to 5-FU is more efficient. Similarly high levels of expression of siRNA or shRNA lead to more efficient suppression of target gene expression. Also for cytokines or polypeptide binding domains (e.g., single chain antibodies (scAbs) and the like) it is usually advantageous to express high levels of the cytokine or binding domain. In addition, in the case that there are mutations in some copies of the vector that inactivate or impair the activity of the vector or transgene, it is advantageous to have multiple copies of the vector in the target cell as this provides a high probability of efficient expression of the intact transgene.

As mentioned above, the integrated DNA intermediate is referred to as a provirus. Prior gene therapy or gene delivery systems use methods and retroviruses that require transcription of the provirus and assembly into infectious virus while in the presence of an appropriate helper virus or in a cell line containing appropriate sequences enabling encapsidation without coincident production of a contaminating helper virus. Similar methods (complementing helper virus or cell line) have been used to generate helper-free viral vector preparations such as those from adenovirus, herpes virus, adeno-associated virus (AAV). As described below, a helper virus is not required for the production of the recombinant retrovirus of the disclosure, since the sequences for encapsidation are provided in the genome thus providing a replication competent retroviral vector for gene delivery or therapy. Similarly, for other replication competent viral vectors such as those derived from adenovirus, herpes viruses, rhabdoviruses, measles, polioviruses, Newcastle Disease Virus, alphaviruses, vaccinia or other pox viruses there is no need for a specific engineered complementing cell line, the viral vector is made by infection of normal host cells, and havesting the resultant virus.

The retroviral genome and the proviral DNA of the disclosure have at least three genes: the gag, the pol, and the env, these genes may be flanked by one or two long terminal repeat (LTR), or in the provirus are flanked by two long terminal repeat (LTR) and sequences containing cis-acting sequences such as psi. The gag gene encodes the internal structural (matrix, capsid, and nucleocapsid) proteins; the pol gene encodes the RNA-directed DNA polymerase (reverse transcriptase), protease and integrase; and the env gene encodes viral envelope glycoproteins. The 5′ and/or 3′ LTRs serve to promote transcription and polyadenylation of the virion RNAs. The LTR contains all other cis-acting sequences necessary for viral replication. Lentiviruses have additional genes including vif, vpr, tat, rev, vpu, nef, and vpx (in HIV-1, HIV-2 and/or SIV). One of skill in the art will recognize that a retroviral genome is an RNA genome and thus reference to any retroviral genome sequence implicitly refers to a sequence wherein “T” is “U”. Thus reference to a gag nucleic acid sequence with a specific sequence containing T, when referring to the retroviral genome implicitly means that the T is replaced with U.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virion) are missing from the viral genome, the result is a cis defect which prevents encapsidation of genomic viral RNA. This type of modified vector is what has typically been used in prior gene delivery systems (i.e., systems lacking elements which are required for encapsidation of the virion) as ‘helper’ elements providing viral proteins in trans that package a non-replicating, but packageable, RNA genome.

The disclosure provides modified retroviral vectors. The modified retroviral vectors can be derived from members of the retroviridae family and be engineered to contain an ENV-2A-SSP-transgene cassette. As mentioned above, the Retroviridae family consists of three groups: the spumaviruses-(or foamy viruses) such as the human foamy virus (HFV); the lentiviruses, as well as visna virus of sheep; and the oncoviruses (although not all viruses within this group are oncogenic).

In one embodiment, the viral vector can be a replication competent retroviral vector capable of infecting only dividing mammalian cells. In one embodiment, a replication competent retroviral vector comprises a 2A peptide or 2A peptide-like sequence just downstream and operably linked to the retroviral envelope and just upstream of a coding sequence for a secretory signal peptide (SSP) which is in-turn linked to a heterologous nucleic acid sequence to be expressed. In certain embodiments, the vector can additionally include an IRES cassette or a polII (or minipromoter) or polIII cassette. The heterologous polynucleotide can encode, e.g., a cytosine deaminase, a nitroreductase, a cytokine, a receptor, an antibody, an antibody fragment, a binding domain (e.g., a non-antibody binding domain or a non-Ig polypeptide) or the like. Where a polIII promoter is included, the vector can further express miRNA, siRNA, or other RNAi sequence.

In another embodiment, the disclosure provides an ENV-2A-SSP-heterologous gene cassette. The cassette can comprise an envelope chosen from one of amphotropic, polytropic, xenotropic, 10A1, GALV, Baboon endogenous virus, RD114, rhabdovirus, alphavirus, measles and influenza virus envelopes. The 2A peptide or 2A peptide-like coding sequence can be any of the sequences set forth in FIG. 1 or 2 operably linked to the C-terminus of the envelope coding sequence. In another embodiment, the 2A peptide or 2A peptide-like coding sequence is linked through a GSG linker sequence (e.g., ggaagcgga (SEQ ID NO:3)). In another embodiment, the GSG-2A peptide or peptide-like coding sequence is linked to an SSP coding sequence. The heterologous gene is operably linked to the C-terminus of the SSP coding sequence. The heterologous gene can be any desired gene to be delivered and expressed in a target cell. In one embodiment, the heterologous gene comprises 500-1500 bp in length or any numerical value therebetween (e.g., 1000 bp, 1100 bp, 1200 bp, 1300 bp, 1400 bp etc.). In another embodiment the heterologous gene comprises >1500 bp in length. In another embodiment, the cassette comprises two heterologous genes separated by a 2A peptide or 2A peptide-like coding sequence upstream of a SSP peptide coding sequence. In yet another embodiment, the cassette can comprise a polynucleotide encoding a 2A peptide or 2A peptide-like sequence operably linked between the C-terminus of the ENV and N-terminus of an SSP sequence which is linked to the N-terminus of a heterologous gene, wherein the heterologous gene is followed by a second cassette comprising an IRES or promoter linked to a second heterologous sequence.

The heterologous nucleic acid sequence is operably linked to a sequence encoding an SSP peptide, which is operably linked and downstream of a 2A peptide or 2A peptide-like sequence. As used herein, the term “heterologous” nucleic acid sequence or transgene refers to (i) a sequence that does not normally exist in a wild-type retrovirus, (ii) a sequence that originates from a foreign species, or (iii) if from the same species, it may be substantially modified from its original form. Alternatively, an unchanged nucleic acid sequence that is not normally expressed in a cell is a heterologous nucleic acid sequence.

Depending upon the intended use of the retroviral vector of the disclosure any number of heterologous polynucleotides or nucleic acid sequences may be inserted into the retroviral vector. For example, for in vitro studies commonly used marker genes or reporter genes may be used, including, antibiotic resistance and fluorescent molecules (e.g., GFP) or luminescent molecules. Additional polynucleotide sequences encoding any desired polypeptide sequence may also be inserted into the vector of the disclosure.

Where in vivo delivery of a heterologous nucleic acid sequence is sought both therapeutic and non-therapeutic sequences may be used. An RRV of the disclosure will comprise at least one cassette comprising an SSP domain. Typically the SSP domain is upstream of a particular polypeptide or protein to be secreted from a cell infected with the RRV. In one embodiment, a biological effect of the SSP can be determined by measuring the amount of secreted polypeptide to which the SSP is attached when translated compared to the same polypeptide lacking the SSP.

In some embodiments a -2A-SSP-transgene cassette can be followed by a minipromoter-cassette, polIII-RNAi cassette or an IRES-cassette. For example, where a minipromoter or polIII cassette is used, the cassette can comprise a heterologous sequence including miRNA, siRNA and the like directed to a particular gene associated with a cell proliferative disorder or other gene-associated disease or disorder. In other embodiments the heterologous gene downstream of an SSP peptide coding sequence or IRES can be a suicide gene (e.g., HSV-tk or PNP or polypeptide having cytosine deaminase activity; either modified or unmodified), a growth factor or a therapeutic protein (e.g., Factor IX, IL2, and the like). Other therapeutic proteins applicable to the disclosure are easily identified in the art. In certain embodiments, where the heterologous gene encodes a protein or polypeptide to be secreted, the heterologous sequence is preceded by a coding sequence for an SSP peptide. For example, where an antibody, antibody fragment, or binding domain is encoded by the heterologous gene, the therapeutic cassette comprises 2A-peptide or peptide-like coding sequence, followed by an SSP coding sequence, which is followed by the heterologous polynucleotide sequence encoding a polypeptide or peptide to be secreted (e.g., an antibody, antibody fragment or binding domain). In certain embodiments, the polypeptide to be secreted is not thymidine kinase. In some embodiments, the RRV can comprise two cassettes, one cassette comprises a polypeptide to be secreted and is preceded by an SSP domain and the second cassette comprises a polypeptide or moiety that is not to be secreted. For example, such dual cassettes can comprise:

-2A-SSP-(polypeptide to be secreted)-(2A or IRES or minipromoter or polIII)-(polypeptide or miRNA)-.

In one embodiment, the heterologous polynucleotide within the vector comprises a cytosine deaminase or thymidine kinase that has been optimized for expression in a human cell. In a further embodiment, the cytosine deaminase comprises a sequence that has been human codon optimized and comprises mutations that increase the cytosine deaminase's stability (e.g., reduced degradation or increased thermo-stability) and/or includes mutations that change a tryptophan codon to a non-tryptophan encoding codon compared to a wild-type cytosine deaminase. In yet another embodiment, the heterologous polynucleotide encodes a fusion construct comprising a polypeptide having cytosine deaminase activity (either human codon optimized or non-optimized, either mutated or non-mutated) operably linked to a polynucleotide encoding a polypeptide having UPRT or OPRT activity.

Antibodies (and fragments thereof) are important class of therapeutics. Their specific binding and functional properties dictate their mode of actions. Most of the FDA approved antibodies are antagonist and have high binding affinity to their targets. Alternatively, the development non-immunoglobulin (non-Ig) scaffold proteins derived from natural endogenous proteins to replace antibodies has been undertaken. The advantages of using non-Ig proteins are that they can achieve high binding affinity and they are relative smaller compared to antibodies and therefore can penetrate tissues more efficiently. They can also be engineered to be multi-valent and/or multiple-target specific.

The disclosure describes the use of natural or artificial signal peptides in RRVs with GSG-linked 2A peptide configuration to produce secreted proteins or polypeptides including, but not limited to, prodrug-activating genes, cytokines or receptor ligands or their analogs, immunoglobulin (Ig) and non-Ig derived proteins. The disclosure also describes other RRV configurations such as ones with an IRES or mini/micro-promoter for expression of the heterologous transgene with a heterologous secretion signal pepetide.

Typically a recombinant replication competent viral vector of the disclosure is modified to include a “cassette”, which typically contain a heterologous gene or sequence to be delivered and expressed in a host cell. The heterologous gene or sequence is operably linked to elements that allow effective expression (e.g., a promoter, IRES or a read-through element that allows transcription and translation of the heterologous sequence).

Transgenes (e.g., the heterologous sequence to be expressed) can be inserted into a retroviral genome in number of locations including into the long-terminal repeats (LTR's), insertion downstream of the envelope and after splice acceptors, fusion with viral gag or pol proteins, internal IRES sequences or small internal promoters downstream of the envelope coding sequence. Insertion of transgenes into LTR's and introduction of extra splice acceptors have led to rapid destabilization of the vector genome, while the IRES and other methods have shown more promise. Expression and the constitution of the transgene can be affected, at least in part, by judicious changes in key sequences such as elimination of cryptic splice acceptors and humanization of transgene sequences (see, e.g., U.S. Pat. No. 8,722,867, the disclosure of which is incorporated herein by reference). The size of a transgene can also have an effect on vector statiblity. For example, in certain vectors as the size of the transgene increases the virus becomes unstable, and rapidly deletes at least part of the heterologous gene or sequence. This limitation is aggravated by the need, in some instances, to include expression enabling sequences such as the IRES (normally about 600 bp, see, e.g., U.S. Pat. No. 8,722,867) or small promoter (normally about 250-300 bp, see, e.g., International Application Publ. No. WO 2014/066700, which is incorporated herein by reference), potentially leaving only 900 to 1200 bp insert of heterologous gene or sequence in, e.g., MLV. Thus, it would be very useful to be able to maximize the available transgene size to include more choice of transgene or multiple transgenes.

Some examples of retroviruses that replicate efficiently in human cells include, amphotropic, polytropic, xenotropic and 10A1 strains of murine leukemia virus (MLV) as well as gibbon ape leukemia virus (GALV), Baboon endogenous virus and the feline virus RD114. Likewise, ecotropic strains of MLV that have been modified to contain a non-ecotropic envelope gene such as amphotropic-pseudotyped RRV can also efficiently replicate in a variety of species and cell types to be treated. However, the retroviral envelope can also be substituted by non-retroviral envelopes such as rhabdovirus, alphavirus, measles or influenza virus envelopes.

Several viruses including picornaviruses and encephalomyocarditis virus encode 2A or 2A-like peptides in their genomes in order to mediate multiple protein expression from a single open reading frame (ORF). 2A peptides are typically about 16-18 amino acid in sequence and share the consensus motif: D[V/I]EXNPGP (SEQ ID NO:1), wherein X is any amino acid. When the 2A peptide is encoded between ORFs in an artificial multicistronic mRNA, it causes the ribosome to halt at the C-terminus of 2A peptide in the translating polypeptide, thus resulting in separation of polypeptides derived from each ORF (Doronina et al., 2008). The separation point is at the C-terminus of 2A, with the first amino acid of the downstream ORF being proline (see, e.g., FIG. 1). The unique features of 2A peptide have led to its utilization as a molecular tool for multiple-protein expression from a single multicistronic mRNA configuration.

2A peptides are present in the viral genome of picornaviridae virus family, such as foot-and-mouth disease virus and equine rhinitis A virus, and other viruses such as the porcine teschovirus-1 and the insect virus Thosea asigna virus (FIG. 1). 2A peptides have near 100% “separation” efficiency in their native contexts, and often have lower “separation” efficiencies when they are introduced into non-native sequences. Other 2A-like sequences found in different classes of virus have also been shown to achieve ˜85% “separation” efficiency in non-native sequences (Donnelly et al., 1997). There is a large number of 2A-like sequences (FIG. 2) that can be be used in the methods and composition of this disclosure for expressing transgenes.

Although 2A sequences have been known to exist for about 20 years, their ability to function in non-native settings has been questioned. In particular the 2A sequences leaves approximately 17-22 extra amino acids on the C terminus of the preceding translated protein and adds a proline onto the N-terminus of the downstream protein, thus, possibly affecting the ability of the preceding protein to function. If the protein requires post-translation modifications in the endoplasmic and Golgi apparatus and/or during the maturation of the virions, as in the case for many viral enveloped proteins (T. Murakami, Mol Biol Int., 2012), there is further risk for functional incompetence for the preceding protein.

Normally, processing of a native MLV envelope protein involves cleavage of the precursor protein Pr85 to gp70 (SU) and p15E (TM) subunit which occurs in infected host cell. Cleavage of Pr85 is required for efficient incorporation of viral envelope protein into the viron during budding from the host cell. As virion buds off from the host cell membrane, the virion undergoes a maturation processes in order to become infectious. One of the processes in MLV virion maturation involves the removal of R-peptide located in the C-terminus of the TM subunit of the envelop protein by viral protease. The 2A peptide except for the last amino acid residue proline (Pro) is expressed downstream of the R-peptide, making the length of R peptide from 16 amino acids to at least 32 amino acids, depending on the sequence of the 2A peptide. Although the length of the R-peptide is lengthened by addition of 2A peptide sequence, theoretically, the 2A peptide will be concurrently removed with the cleavage of R peptide, resulting in a functional envelop protein.

If the envelope sequence is non-functional or attenuated, the viral vector is likely not to be useful. There have been attempts to use a particular 2A sequence (from porcine teschovirus-1, “P2A”) in a retroviral construct with a particular envelope (ecotropic) that infects only mice (S. Stavrou et al., PLoS Pathog 10(5):e1004145, 2014; and E. P. Browne, J. Virol. 89:155-64, 2015). However, these viruses do not infect human cells and there is no expectation that the general protein processing problem has been solved. Moreover, the viruses so constructed were designed to express genes that facilitate viral replication in vivo, rather than achieves a therapeutic effect.

In some instances, it is desireable to have a protein or polypeptide delivered by a recombinant retroviral vector to a host cell to be secreted from the infected cell. That is, an RRV carrying a cassette containing a heterologous polynucleotide encoding a polypeptide or protein is engineered to be secreted from the infected target cell wherein the resulting RRV's proviral DNA is incorporated into the target cell's genome. As mentioned above, a secretory signal peptide can be engineered upstream of the polypeptide or protein in order to cause the polypeptide or protein to be secreted from the cell. In such instances the secretory signal peptide coding sequence is engineered to be located between the 2A- or 2A-like-peptide and the polypeptide or protein to be secreted. Thus, a cassette in such an RRV would be located between the env coding sequence and the 3′ LTR having the general structure: -(env) -(2A)-(SSP)-(polypeptide or protein)-(LTR). As can be seen from the foregoing general structure, the cassette can be viewed as modular and various 2A or 2A-like sequences, SSP sequences and polypeptide or protein sequences can be changed/shuffled.

Monoclonal antibodies remain the mainstream for human therapeutics in diagnostics and cancer therapy. They have long serum half-life, bivalency and immune effector functions. Despite their partial or fully human nature which minimizes immunogenicity, monoclonal antibodies are complex protein with multiple domains that require proper disulfide bond formation and glycosylation process and thus its production is limited in eukaryotic cells which also have limited scalability. Another important potential limitation of monoclonal antibodies is that it is believed that a full antibody of 150 KDa in size may also have limited tissue penetration and intracellular accessibility. Some of these limitations have been overcome by developing fragmented antibodies such as single-chain variable fragment (scFv) or Fab. Further developments have also utilized binding proteins of camelids and cartilaginous fish, which comprise heavy-chain only isotypes devoid of light chains.

Non-immunoglobulin (Ig) scaffold proteins have been developed for biotherapeutics using randomization strategies to identify antigen-binding sequences (U. H Wiedle et al., Cancer Genomics & Proteomics 10:155-168, 2013; K. Skrlec et al., Trends in Biotechnology, 33:408-418, 2015). Non-Ig scaffold proteins are domain-derived subunits of natural proteins from human and other species or are artificial and their size range from 6-20 kDa and can be expressed from a single polypeptide. They possess surface-exposed loops or amino acids in alpha-helical or beta sheet framework that can tolerate insertion, deletion and substitutions which via randomization, phage display screening and affinity maturation processes resulted in antigen-binding scaffold proteins that can function as antagonists or agonists. To date, there are more than 50 different classes of non-Ig scaffold proteins that have been identified and developed for therapeutics as scaffold binders. Due to their size, one major challenge these proteins face are fast renal clearance leading to short half-life in circulation. One common solution to improve the half-life of these non-Ig scaffold proteins involve using fusion proteins containing scaffold proteins linked to the Fc region of IgG. Another challenge is that they normally have lower binding affinity (KD 1-100 nM) than monoclonal antibodies and are associated with fast dissociation rates. Genetic modification of these scaffold proteins to include multimerization domain may increase steric hindrance-mediated blocking or avidity where which in certain signaling pathways can lead to biological functions and therapeutic effects. Multiple methods have been proposed and at least partially tested using fusion proteins containing scaffold proteins linked to the Fc region of IgG or containing two repeat units of scaffold proteins linked by a linker to generate dimers. In addition to linker peptides and the Fc region of IgG, dimer-, trimer- and pentamer-multimerization domains have been utilized to express ectodomain of desired proteins that naturally occur in oligomeric state or to strengthen protein-protein interaction.

The disclosure provides compositions and methods that use binding domains that comprise combinations of heavy and/or light chain CDRs linked by scaffold domains (e.g., Adhiron scaffold; scaffolds from human stefin A—see, EP22792058B1 and WO2019/008335 the disclosures of which are incorporated herein by reference). In some embodiments, the coding sequence for the binding domain(s) is operably linked and downstream of a 2A or 2A-like peptide coding sequence. In another embodiment, the coding sequence for the binding domain(s) is operably linked to a coding sequence of a secretory signal peptide. In still another embodiment, the coding sequence for the binding domain(s) is operably linked and downstream of a 2A or 2A-like peptide coding sequence, which is inturn linked to a secretory signal peptide coding sequence such that a nucleic acid cassette has the general structure: -2A-SSP-binding domain-. In other embodiments, the disclosure provide composition and use of the Fc region of IgG, portion of Fc region of IgA and IgM, glycine-serine linkers and multimerization domain to form oligomeric antigen-binding scaffold proteins. Any of the foregoing can be used in combination with an RRV having sequence optimization to minimize Apobec3-mediated hypermutations and thus to enhance protein stability and/or avidity as well as expression for potential better biological functions and therapeutic effects. The disclosure also provides expression vectors and method of use, in particular viral vectors that have high tumor-targeting specificity, to deliver therapeutic payload in the tumor microenvironment to offset rapid clearance of these antigen-binding non-Ig scaffold proteins in circulation and minimize off-target effects and toxicity when administered intravenously. Tables 1, 2, 3, 4 and 5 provide sequences useful in the compositions and methods of the disclosure. Please note that “T” can be “U” in the following nucleic acid sequences as RNA is contemplated by the disclosure.

TABLE 1
Amino acid sequence of some non-Ig scaffold
proteins that can function as antigen-binding proteins

| Scaffold | Amino Acid Sequence (SEQ ID NO:) |
|---|---|
| Adnectins | VSDVPRKLEVVAATPTSLLISWDAPAVTVRYYRITYGETGGNSPVQEFTVPGSKS |
| (10Fn3) | TATISGLKPGVDYTITVYAVTGRGDSPASSKPISNYRTALE (SEQ ID |
| | NO: 127) |
| Adnectin 1 | VSDVPRKLEVVAATPTSLLISWDSGRGSYRYYRITYGETGGNSPVQEFTVPGPVH |
| | TATISGLKPGVDYTITVYAVTDHKPHADGPHTYHESPISNYRTALE (SEQ ID |
| | NO: 129) |
| Adnectin 2 | VSDVPRKLEVVAATPTSLLISWEHDYPYRRYYRITYGETGGNSPVQEFTVPKDVD |
| | TATISGLKPGVDYTITVYAVTSSYKYDMQYSPISNYRTALE (SEQ ID |
| | NO: 131) |
| Pronectins | SGPVEVFITETPSQPNSHPIQWNAPQPSHISKYILRWRPKNSVGRWKEATIPGHL |
| (1Fn3) | NSYTIKGLKPGVVYEGQLISIQQYGHQEVTRFDFTTT (SEQ ID NO: 133) |
| Pronectins | SPLVATSESVTEITASSFVVSWVSASDTVSGFRVEYELSEEGDEPQYLDLPSTAT |
| (2Fn3) | SVNIPDLLPGRKYIVNVYQ1SEDGEQSLILSTSQTT (SEQ ID NO: 135) |
| Pronectins | APDAPPDPTVDQVDDTSIVVRWSRPQAPITGYRIVYSPSVEGSSTELNLPETANS |
| (3Fn3) | VTLSDLQPGVQYNITIYAVEENQESTPVVIQQETTGTPR (SEQ ID |
| | NO: 137) |
| Pronectins | TVPSPRDLQFVEVTDVKVTIMWTPPESAVTGYRVDVIPVNLPGEHGQRLPISRNT |
| (4Fn3) | FAEVTGLSPGVTYYFKVFAVSHGRESKPLTAQQTT (SEQ ID NO: 139) |
| Pronectins | KLDAPTNLQFVNETDSTVLVRWTPPRAQITGYRLTVGLTRRGQPRQYNVGPSVSK |
| (5Fn3) | YPLRNLQPASEYTVSLVAIKGNQESPKATGVFTTL (SEQ ID NO: 141) |
| Pronectins | QPGSSIPPYNTEVTETTIVITWTPAPRlGFKLGVRPSQGGEAPREVTSDSGSVVS |
| (6Fn3) | GLTPGVEYVYTIQVLRDGQERDAPIVNKVVT (SEQ ID NO: 143) |
| Pronectins | PLSPPTNLHLEANPDTGVLTVSWERSTTPDITGYRITTTPTNGQQGNSLEEVVHA |
| (7Fn3) | DQSSCTFDNLSPGLEYNVSVYTVKDDKESVPISDTIIP (SEQ ID NO: 145) |
| Pronectins | AVPPPTDLRFTNIGPDTMRVTWAPPPSIDLTNFLVRYSPVKNEEDVAELSISPSD |
| (8Fn3) | NAVVLTNLLPGTEYVVSVSSVYEQHESTPLRGRQKT (SEQ ID NO: 147) |
| Pronectins | GLDSPTGIDFSDITANSFTVHWIAPRATITGYRIRHHPEHFSGRPREDRVPHSRN |
| (9Fn3) | SITLTNLTPGTEYVVSIVALNGREESPLLIGQQST (SEQ ID NO: 149) |
| Pronectins | VSDVPRDLVVAATPTSLLISWDAPAVTVRYYRITYGETGGNSPVQEFTVPGSKST |
| (10Fn3) | ATISGLKPGVDYTITVYAVTGRGDSPASSKPISINYRT (SEQ ID NO: 151) |
| Pronectins | EIDKPSQMQVTDVQDNSISVKWLPSSSPVTGYRVTTTPKNGPGPTKTKTAGPDQT |
| (11Fn3) | EMTIEGLQPTVEYVVSVYAQNPSGESQPLVQTAVT (SEQ ID NO: 153) |
| Pronectins | NIDRPKGLAFTDVDVDSIKIAWESPQGQVSRYRVTYSSPEDGIHELFPAPDGEED |
| (12Fn3) | TAELQGLRPGSEYTVSVVALHDDMESQPLIGTQST (SEQ ID NO: 155) |
| Pronectins | AIPAPTDLKFTQVTPTSLSAQWTPPNVQLTGYRVRVTPKEKTGPMKEINLAPDSS |
| (13Fn3) | SVVVSGLMVATKYEVSVYALKDTLTSRPAQGVVTTLE (SEQ ID NO: 157) |
| Pronectins | NVSPPRRARVTDATETTITISWRTKTETITGFQVDAVPANGQTPIQRTIKPDVRS |
| (14Fn3) | YTITGLQPGTDYKIYLYTLNDNARSSVVIDAST (SEQ ID NO: 159) |
| Pronectins | AIDAPSNLRFLATTPNSLLVSWQPPRARITGYIIKYEKPGSPPREVVPRPRPGVT |
| (15Fn3) | EATITGLEPGTEYTIYVIALKNNQKSEPLIGRKKT (SEQ ID NO: 161) |
| Pronectins | PGLNPNASTGQEALSQTTISWAPFQDTSEYIISCHPVGTDEEPLQFRVPGTSTSA |
| (16Fn3) | TLTGLTRGATYNIIVEALKDQQRHKVREEVVTV (SEQ ID NO: 163) |
| Adhiron | ATGVRAVPGNENSLEIEELARFAVDEHNKKENALLEFVRVVKAKEQVVAGTMYYL |
| | TLEAKDGGKKKLYEAKVWVKPWENFKELQEFKPVGDA (SEQ ID NO: 165) |
| Affibodies | VDNKFNKEQQNAFYEILHLPNLNEEQRNAFIQSLKDDPSQSANLLAEAKKLNDAQ |
| | APK (SEQ ID NO: 167) |
| Affilins (γ-B- | GKITFYEDRAFQGRSYECTTDCPNLQPYFSRCNSIRVESGCWMIYERPNYQGHQY |
| Crystallin) | FLRRGEYPDYQQWMGLSDSIRSCCLIPPHSGAYRMKIYDRDELRGQMSELTDDCI |
| | SVQDRFHLTEIHSLNVLEGSWILYEMPNYRGRQYLLRPGEYRRFLDWGAPNAKVG |
| | SLRRVMDLY (SEQ ID NO: 169) |
| Affimers | MIPRGLSEAKPATPEIQEIVDKVKPQLEEKTNETYGKLEAVQYKTQVLASTNYYI |
| | KVRAGDNKYMHLKVFNGPPGQNADRVLTGYQVDKNKDDELTGF (SEQ ID |
| | NO: 171) |
| Anticalin | IASDEEIQDVSGTWYLKAMTVDREFPEMNLESVTPMTLTTLEGGNLEAKVTMLIS |
| (lipocalin | GRCQEVKAVLEKTDEPGKYTADGGKHVAYIIRSHVKDHYIFYSEGELHGKPVRGV |
| Lcn1) | KLVGRDPKNNLEALLDFEKAAGARGLSTESILIPRQSETCSPGS (SEQ ID |
| | NO: 173) |
| Anticalins | QDSTSDLIPAPPLSKVPLQQNFQDNQFQGKWYVVGLAGNAILREDKDPQKMYATI |
| (lipocalin | YELKEDKSYNVTSVLFRKKKCDYWIRTFVPGCQPGEFTLGNIKSYPGLTSYLVRV |
| Lcn2) | VSTNYNQHAMVFFKKVSQNREYFKITLYGRTKELTSELKENFIRFSKSLGLPENH |
| | IVFPVPIDQCIDG (SEQ ID NO: 175) |
| Avimers (C426) | CESGEFQCHSTGRCIPQEWVCDGDNDCEDSSDEAPDLCASAEPTCPSGEFQCRST |
| targeting c- | NRCIPETWLCDGDNDCEDGSDEESCTPPT (SEQ ID NO: 177) |
| MET | |
| Centyrins | LPAPKNLVVSEVTEDSARLSWTAPDAAFDSFLIGYGESEKVGEAIVLTVPGSERS |
| (Fn3 domain of | YDLTGLKPGTEYTVSIYGVKGGHRSNPLSAIFTT (SEQ ID NO: 179) |
| Tenascin) | |
| Cys- | CSPSGAICSGFGPPEQCCSAGCVLNRRARSWRCQ (SEQ ID NO: 212) |
| knots/Knottin | |
| (SOTI Var. 1) | |
| cleavage by | |
| AEP-like | |
| ligase in | |
| acidic is | |
| required | |
| Cys- | CSPSGAICSGFGPPEQCCSAGACVPHPILRIFVCQ (SEQ ID NO: 213) |
| knots/Knottin | |
| (SOTI-III) | |
| Kalata B1 | GLPVCGETCVGGTCNTPGCTCSWPVCTRN (SEQ ID NO: 214/215) |
| Kalata B2 | GLPVCGETCFGGTCNTPGCSCTWPICTRD (SEQ ID NO: 216) |
| MCoTI-I | GGVCPKILQRCRRDSDCPGACICRGNGYCGSGSD (SEQ ID NO: 217) |
| MCoTI-II | GGVCPKILKKCRRDSDCPGACICRGNGYCGSGSD (SEQ ID NO: 218) |
| Kunitz | VREVCSEQAETGPCRAMISRWYFDVTEGKCAPFFYGGCCGGNRNNFDTEEYCMAV |
| domain/BPTI | CG (SEQ ID NO: 181) |
| Obodies | EIMDAAEDYAKERYGISSMIQSQEKPDRVLVRVRDLTIQKADEVVWVRARVHTSR |
| (human AspRS) | AKGKQCFLVLRQQQFNVQALVAVGDHASKQMVKFAANINKESIVDVEGVVRKVNQ |
| | KIGSCTQQDVELHVQKIYVISLAEPRLPLQLDDAVRPEAEGEEEGRATVNQDTRL |
| | DNRVIDL (SEQ ID NO: 183) |
| Tn3A | AIEVKDVTDTTALITWSDEFGHDYDGCELTYGIKDVPGDRTTIDLWWHSAWYSIG |
| | NLKPDTEDVSLICYTDQEAGNPAKETFTTGLVPR (SEQ ID NO: 185) |
| Tn3B | AIEVEDVTDTTALITWTNRSSYSNLHGCELAYGIKDVPGDRTTIDLNQPYVHYSI |
| | GNLKPDTEYEVSLICLTTDGTYNNPAKETFTTGLVPR (SEQ ID NO: 187) |
| Hckomers | TLFVALYDYEARTEDELSFHKGEKFQILNSSEGDWWEARDSLTTGETGYIPSNYV |
| | APVD (SEQ ID NO: 189) |
| NPHP1 | EEYIAVGDFDTAQQVGDLTFKKGEILLVIEKKPDGWWIAKDAKGNEGLVPRTYLE |
| | PYS (SEQ ID NO: 191) |
| Tec | EIVVAMYDFQAAEGHDLRLERQEYLILEKNDVHWWRARDKYGNEGYIPSNYVTGK |
| | K (SEQ ID NO: 193) |
| Hck | IIVVALYDYEAIHHEDLSFQKGDQMVVLEESGEWWKARSLATRKEGYIPSNYVAR |
| | VD (SEQ ID NO: 195) |
| Amph | YKVETLHDFEAANSDELTLQRGDVVLVVPSDSEADQDAGWLVGVKESDWLQYRDL |
| | ATYKGLFPENFTRRLD (SEQ ID NO: 197) |
| RIMBP#3 | KIMIAALDYDPGDGQMGGQGKGRLALRAGDVVMVYGPMDDQGFYYGELGGHRGLV |
| | PAHLLDHMS (SEQ ID NO: 199) |
| IRIKS | QKVKTIFPHTAGSNKTLLSFAQGDVITLLIPEEKDGWLYGEHDVSKARGWFPSSY |
| | TKLLE (SEQ ID NO: 201) |
| SNX33 | LKGRALYDFHSENKEEISIQQDEDLVIFSETSLDGWLQGQNSRGETGLFPASYVE |
| | IVR (SEQ ID NO: 203) |
| Eps8L1 | KWVLCNYDFQARNSSELSVKQRDVLEVLDDSRKWWKVRDPAGQEGYVPYNILTPY |
| | P (SEQ ID NO: 205) |
| FISH#5 | DVYVSIADYEGDEETAGFQEGVSMEVLERNPNGWWYCQILDGVKPFKGWVPSNYL |
| | EKKN (SEQ ID NO: 207) |
| CMS#1 | VDYIVEYDYDAVHDDELTIRVGEIIRNVKKLQEEGWLEGELNGRRGMFPDNFVKE |
| | IK (SEQ ID NO: 209) |
| OSTF1 | KVFRALYTFEPRTPDELYFEEGDIIYITDMSDTNWWKGTSKGRTGLIPSNYVAEQ |
| | A (SEQ ID NO: 211) |

TABLE 2
Nucleic acid sequence of non-Ig scaffold proteins
that can function as antigen-binding proteins

| Scaffold | Nucleic Acid Sequence (SEQ ID NO:) |
|---|---|
| Adnectins (10Fn3) | GTGAGCGACGTGCCCAGAAAGCTGGAGGTGGTGGCCGCCACCCCCACCAGC |
| | CTGCTGATCAGCTGGGACGCCCCCGCCGTGACCGTGAGATACTACAGAATC |
| | ACCTACGGCGAGACCGGCGGCAACAGCCCCGTGCAGGAGTTCACCGTGCCC |
| | GGCAGCAAGAGCACCGCCACCATCAGCGGCCTGAAGCCCGGCGTGGACTAC |
| | ACCATCACCGTGTACGCCGTGACCGGCAGAGGCGACAGCCCCGCCAGCAGC |
| | AAGCCCATCAGCAACTACAGAACCGCCCTGGAG (SEQ ID NO: 126) |
| Adnectin 1 | GTGAGCGACGTGCCCAGAAAGCTGGAGGTGGTGGCCGCCACCCCCACCAGC |
| | CTGCTGATCAGCTGGGACAGCGGCAGAGGCAGCTACAGATACTACAGAATC |
| | ACCTACGGCGAGACCGGCGGCAACAGCCCCGTGCAGGAGTTCACCGTGCCC |
| | GGCCCCGTGCACACCGCCACCATCAGCGGCCTGAAGCCCGGCGTGGACTAC |
| | ACCATCACCGTGTACGCCGTGACCGACCACAAGCCCCACGCCGACGGCCCC |
| | CACACCTACCACGAGAGCCCCATCAGCAACTACAGAACCGCCCTGGAG |
| | (SEQ ID NO: 128) |
| Adnectin 2 | GTGAGCGACGTGCCCAGAAAGCTGGAGGTGGTGGCCGCCACCCCCACCAGC |
| | CTGCTGATCAGCTGGGAGCACGACTACCCCTACAGAAGATACTACAGAATC |
| | ACCTACGGCGAGACCGGCGGCAACAGCCCCGTGCAGGAGTTCACCGTGCCC |
| | AAGGACGTGGACACCGCCACCATCAGCGGCCTGAAGCCCGGCGTGGACTAC |
| | ACCATCACCGTGTACGCCGTGACCAGCAGCTACAAGTACGACATGCAGTAC |
| | AGCCCCATCAGCAACTACAGAACCGCCCTGGAG (SEQ ID NO: 130) |
| Pronectins (1Fn3) | AGCGGCCCCGTGGAGGTGTTCATCACCGAGACCCCCAGCCAGCCCAACAGC |
| | CACCCCATCCAGTGGAACGCCCCCCAGCCCAGCCACATCAGCAAGTACATC |
| | CTGAGATGGAGACCCAAGAACAGCGTGGGCAGATGGAAGGAGGCCACCATC |
| | CCCGGCCACCTGAACAGCTACACCATCAAGGGCCTGAAGCCCGGCGTGGTG |
| | TACGAGGGCCAGCTGATCAGCATCCAGCAGTACGGCCACCAGGAGGTGACC |
| | AGATTCGACTTCACCACCACC (SEQ ID NO: 132) |
| Pronectins (2Fn3) | AGCCCCCTGGTGGCCACCAGCGAGAGCGTGACCGAGATCACCGCCAGCAGC |
| | TTCGTGGTGAGCTGGGTGAGCGCCAGCGACACCGTGAGCGGCTTCAGAGTG |
| | GAGTACGAGCTGAGCGAGGAGGGCGACGAGCCCCAGTACCTGGACCTGCCC |
| | AGCACCGCCACCAGCGTGAACATCCCCGACCTGCTGCCCGGCAGAAAGTAC |
| | ATCGTGAACGTGTACCAGAGCGAGGACGGCGAGCAGAGCCTGATCCTGAGC |
| | ACCAGCCAGACCACC (SEQ ID NO: 134) |
| Pronectins (3Fn3) | GCCCCCGACGCCCCCCCCGACCCCACCGTGGACCAGGTGGACGACACCAGC |
| | ATCGTGGTGAGATGGAGCAGACCCCAGGCCCCCATCACCGGCTACAGAATC |
| | GTGTACAGCCCCAGCGTGGAGGGCAGCAGCACCGAGCTGAACCTGCCCGAG |
| | ACCGCCAACAGCGTGACCCTGAGCGACCTGCAGCCCGGCGTGCAGTACAAC |
| | ATCACCATCTACGCCGTGGAGGAGAACCAGGAGAGCACCCCCGTGGTGATC |
| | CAGCAGGAGACCACCGGCACCCCCAGA (SEQ ID NO: 136) |
| Pronectins (4Fn3) | ACCGTGCCCAGCCCCAGAGACCTGCAGTTCGTGGAGGTGACCGACGTGAAG |
| | GTGACCATCATGTGGACCCCCCCCGAGAGCGCCGTGACCGGCTACAGAGTG |
| | GACGTGATCCCCGTGAACCTGCCCGGCGAGCACGGCCAGAGACTGCCCATC |
| | AGCAGAAACACCTTCGCCGAGGTGACCGGCCTGAGCCCCGGCGTGACCTAC |
| | TACTTCAAGGTGTTCGCCGTGAGCCACGGCAGAGAGAGCAAGCCCCTGACC |
| | GCCCAGCAGACCACC (SEQ ID NO: 138) |
| Pronectins (5Fn3) | AAGCTGGACGCCCCCACCAACCTGCAGTTCGTGAACGAGACCGACAGCACC |
| | GTGCTGGTGAGATGGACCCCCCCCAGAGCCCAGATCACCGGCTACAGACTG |
| | ACCGTGGGCCTGACCAGAAGAGGCCAGCCCAGACAGTACAACGTGGGCCCC |
| | AGCGTGAGCAAGTACCCCCTGAGAAACCTGCAGCCCGCCAGCGAGTACACC |
| | GTGAGCCTGGTGGCCATCAAGGGCAACCAGGAGAGCCCCAAGGCCACCGGC |
| | GTGTTCACCACCCTG (SEQ ID NO: 140) |
| Pronectins (6Fn3) | CAGCCCGGCAGCAGCATCCCCCCCTACAACACCGAGGTGACCGAGACCACC |
| | ATCGTGATCACCTGGACCCCCGCCCCCAGACTGGGCTTCAAGCTGGGCGTG |
| | AGACCCAGCCAGGGCGGCGAGGCCCCCAGAGAGGTGACCAGCGACAGCGGC |
| | AGCGTGGTGAGCGGCCTGACCCCCGGCGTGGAGTACGTGTACACCATCCAG |
| | GTGCTGAGAGACGGCCAGGAGAGAGACGCCCCCATCGTGAACAAGGTGGTG |
| | ACC (SEQ ID NO: 142) |
| Pronectins (7Fn3) | CCCCTGAGCCCCCCCACCAACCTGCACCTGGAGGCCAACCCCGACACCGGC |
| | GTGCTGACCGTGAGCTGGGAGAGAAGCACCACCCCCGACATCACCGGCTAC |
| | AGAATCACCACCACCCCCACCAACGGCCAGCAGGGCAACAGCCTGGAGGAG |
| | GTGGTGCACGCCGACCAGAGCAGCTGCACCTTCGACAACCTGAGCCCCGGC |
| | CTGGAGTACAACGTGAGCGTGTACACCGTGAAGGACGACAAGGAGAGCGTG |
| | CCCATCAGCGACACCATCATCCCCTGA (SEQ ID NO: 144) |
| Pronectins (8Fn3) | GCCGTGCCCCCCCCCACCGACCTGAGATTCACCAACATCGGCCCCGACACC |
| | ATGAGAGTGACCTGGGCCCCCCCCCCCAGCATCGACCTGACCAACTTCCTG |
| | GTGAGATACAGCCCCGTGAAGAACGAGGAGGACGTGGCCGAGCTGAGCATC |
| | AGCCCCAGCGACAACGCCGTGGTGCTGACCAACCTGCTGCCCGGCACCGAG |
| | TACGTGGTGAGCGTGAGCAGCGTGTACGAGCAGCACGAGAGCACCCCCCTG |
| | AGAGGCAGACAGAAGACCTGA (SEQ ID NO: 146) |
| Pronectins (9Fn3) | GGCCTGGACAGCCCCACCGGCATCGACTTCAGCGACATCACCGCCAACAGC |
| | TTCACCGTGCACTGGATCGCCCCCAGAGCCACCATCACCGGCTACAGAATC |
| | AGACACCACCCCGAGCACTTCAGCGGCAGACCCAGAGAGGACAGAGTGCCC |
| | CACAGCAGAAACAGCATCACCCTGACCAACCTGACCCCCGGCACCGAGTAC |
| | GTGGTGAGCATCGTGGCCCTGAACGGCAGAGAGGAGAGCCCCCTGCTGATC |
| | GGCCAGCAGAGCACCTGA (SEQ ID NO: 148) |
| Pronectins | GTGAGCGACGTGCCCAGAGACCTGGTGGTGGCCGCCACCCCCACCAGCCTG |
| (10Fn3) | CTGATCAGCTGGGACGCCCCCGCCGTGACCGTGAGATACTACAGAATCACC |
| | TACGGCGAGACCGGCGGCAACAGCCCCGTGCAGGAGTTCACCGTGCCCGGC |
| | AGCAAGAGCACCGCCACCATCAGCGGCCTGAAGCCCGGCGTGGACTACACC |
| | ATCACCGTGTACGCCGTGACCGGCAGAGGCGACAGCCCCGCCAGCAGCAAG |
| | CCCATCAGCATCAACTACAGAACC (SEQ ID NO: 150) |
| Pronectins | GAGATCGACAAGCCCAGCCAGATGCAGGTGACCGACGTGCAGGACAACAGC |
| (11Fn3) | ATCAGCGTGAAGTGGCTGCCCAGCAGCAGCCCCGTGACCGGCTACAGAGTG |
| | ACCACCACCCCCAAGAACGGCCCCGGCCCCACCAAGACCAAGACCGCCGGC |
| | CCCGACCAGACCGAGATGACCATCGAGGGCCTGCAGCCCACCGTGGAGTAC |
| | GTGGTGAGCGTGTACGCCCAGAACCCCAGCGGCGAGAGCCAGCCCCTGGTG |
| | CAGACCGCCGTGACC (SEQ ID NO: 152) |
| Pronectins | AACATCGACAGACCCAAGGGCCTGGCCTTCACCGACGTGGACGTGGACAGC |
| (12Fn3) | ATCAAGATCGCCTGGGAGAGCCCCCAGGGCCAGGTGAGCAGATACAGAGTG |
| | ACCTACAGCAGCCCCGAGGACGGCATCCACGAGCTGTTCCCCGCCCCCGAC |
| | GGCGAGGAGGACACCGCCGAGCTGCAGGGCCTGAGACCCGGCAGCGAGTAC |
| | ACCGTGAGCGTGGTGGCCCTGCACGACGACATGGAGAGCCAGCCCCTGATC |
| | GGCACCCAGAGCACCTGA (SEQ ID NO: 154) |
| Pronectins | GCCATCCCCGCCCCCACCGACCTGAAGTTCACCCAGGTGACCCCCACCAGC |
| (13Fn3) | CTGAGCGCCCAGTGGACCCCCCCCAACGTGCAGCTGACCGGCTACAGAGTG |
| | AGAGTGACCCCCAAGGAGAAGACCGGCCCCATGAAGGAGATCAACCTGGCC |
| | CCCGACAGCAGCAGCGTGGTGGTGAGCGGCCTGATGGTGGCCACCAAGTAC |
| | GAGGTGAGCGTGTACGCCCTGAAGGACACCCTGACCAGCAGACCCGCCCAG |
| | GGCGTGGTGACCACCCTGGAG (SEQ ID NO: 156) |
| Pronectins | AACGTGAGCCCCCCCAGAAGAGCCAGAGTGACCGACGCCACCGAGACCACC |
| (14Fn3) | ATCACCATCAGCTGGAGAACCAAGACCGAGACCATCACCGGCTTCCAGGTG |
| | GACGCCGTGCCCGCCAACGGCCAGACCCCCATCCAGAGAACCATCAAGCCC |
| | GACGTGAGAAGCTACACCATCACCGGCCTGCAGCCCGGCACCGACTACAAG |
| | ATCTACCTGTACACCCTGAACGACAACGCCAGAAGCAGCGTGGTGATCGAC |
| | GCCAGCACC (SEQ ID NO: 158) |
| Pronectins | GCCATCGACGCCCCCAGCAACCTGAGATTCCTGGCCACCACCCCCAACAGC |
| (15Fn3) | CTGCTGGTGAGCTGGCAGCCCCCCAGAGCCAGAATCACCGGCTACATCATC |
| | AAGTACGAGAAGCCCGGCAGCCCCCCCAGAGAGGTGGTGCCCAGACCCAGA |
| | CCCGGCGTGACCGAGGCCACCATCACCGGCCTGGAGCCCGGCACCGAGTAC |
| | ACCATCTACGTGATCGCCCTGAAGAACAACCAGAAGAGCGAGCCCCTGATC |
| | GGCAGAAAGAAGACC (SEQ ID NO: 160) |
| Pronectins | CCCGGCCTGAACCCCAACGCCAGCACCGGCCAGGAGGCCCTGAGCCAGACC |
| (16Fn3) | ACCATCAGCTGGGCCCCCTTCCAGGACACCAGCGAGTACATCATCAGCTGC |
| | CACCCCGTGGGCACCGACGAGGAGCCCCTGCAGTTCAGAGTGCCCGGCACC |
| | AGCACCAGCGCCACCCTGACCGGCCTGACCAGAGGCGCCACCTACAACATC |
| | ATCGTGGAGGCCCTGAAGGACCAGCAGAGACACAAGGTGAGAGAGGAGGTG |
| | GTGACCGTG (SEQ ID NO: 162) |
| Adhiron | GCCACCGGCGTGAGAGCCGTGCCCGGCAACGAGAACAGCCTGGAGATCGAG |
| | GAGCTGGCCAGATTCGCCGTGGACGAGCACAACAAGAAGGAGAACGCCCTG |
| | CTGGAGTTCGTGAGAGTGGTGAAGGCCAAGGAGCAGGTGGTGGCCGGCACC |
| | ATGTACTACCTGACCCTGGAGGCCAAGGACGGCGGCAAGAAGAAGCTGTAC |
| | GAGGCCAAGGTGTGGGTGAAGCCCTGGGAGAACTTCAAGGAGCTGCAGGAG |
| | TTCAAGCCCGTGGGCGACGCC (SEQ ID NO: 164) |
| Affibodies | GTGGACAACAAGTTCAACAAGGAGCAGCAGAACGCCTTCTACGAGATCCTG |
| | CACCTGCCCAACCTGAACGAGGAGCAGAGAAACGCCTTCATCCAGAGCCTG |
| | AAGGACGACCCCAGCCAGAGCGCCAACCTGCTGGCCGAGGCCAAGAAGCTG |
| | AACGACGCCCAGGCCCCCAAGTGA (SEQ ID NO: 166) |
| Affilins | GGCAAGATCACCTTCTACGAGGACAGAGCCTTCCAGGGCAGAAGCTACGAG |
| (γ-B Crystallin) | TGCACCACCGACTGCCCCAACCTGCAGCCCTACTTCAGCAGATGCAACAGC |
| | ATCAGAGTGGAGAGCGGCTGCTGGATGATCTACGAGAGACCCAACTACCAG |
| | GGCCACCAGTACTTCCTGAGAAGAGGCGAGTACCCCGACTACCAGCAGTGG |
| | ATGGGCCTGAGCGACAGCATCAGAAGCTGCTGCCTGATCCCCCCCCACAGC |
| | GGCGCCTACAGAATGAAGATCTACGACAGAGACGAGCTGAGAGGCCAGATG |
| | AGCGAGCTGACCGACGACTGCATCAGCGTGCAGGACAGATTCCACCTGACC |
| | GAGATCCACAGCCTGAACGTGCTGGAGGGCAGCTGGATCCTGTACGAGATG |
| | CCCAACTACAGAGGCAGACAGTACCTGCTGAGACCCGGCGAGTACAGAAGA |
| | TTCCTGGACTGGGGCGCCCCCAACGCCAAGGTGGGCAGCCTGAGAAGAGTG |
| | ATGGACCTGTAC (SEQ ID NO: 168) |
| Affimers | ATGATCCCCAGAGGCCTGAGCGAGGCCAAGCCCGCCACCCCCGAGATCCAG |
| | GAGATCGTGGACAAGGTGAAGCCCCAGCTGGAGGAGAAGACCAACGAGACC |
| | TACGGCAAGCTGGAGGCCGTGCAGTACAAGACCCAGGTGCTGGCCAGCACC |
| | AACTACTACATCAAGGTGAGAGCCGGCGACAACAAGTACATGCACCTGAAG |
| | GTGTTCAACGGCCCCCCCGGCCAGAACGCCGACAGAGTGCTGACCGGCTAC |
| | CAGGTGGACAAGAACAAGGACGACGAGCTGACCGGCTTC (SEQ ID |
| | NO: 170) |
| Anticalin | ATCGCCAGCGACGAGGAGATCCAGGACGTGAGCGGCACCTGGTACCTGAAG |
| (lipocalin Lcn1) | GCCATGACCGTGGACAGAGAGTTCCCCGAGATGAACCTGGAGAGCGTGACC |
| | CCCATGACCCTGACCACCCTGGAGGGCGGCAACCTGGAGGCCAAGGTGACC |
| | ATGCTGATCAGCGGCAGATGCCAGGAGGTGAAGGCCGTGCTGGAGAAGACC |
| | GACGAGCCCGGCAAGTACACCGCCGACGGCGGCAAGCACGTGGCCTACATC |
| | ATCAGAAGCCACGTGAAGGACCACTACATCTTCTACAGCGAGGGCGAGCTG |
| | CACGGCAAGCCCGTGAGAGGCGTGAAGCTGGTGGGCAGAGACCCCAAGAAC |
| | AACCTGGAGGCCCTGCTGGACTTCGAGAAGGCCGCCGGCGCCAGAGGCCTG |
| | AGCACCGAGAGCATCCTGATCCCCAGACAGAGCGAGACCTGCAGCCCCGGC |
| | AGC (SEQ ID NO: 172) |
| Anticalins | CAGGACAGCACCAGCGACCTGATCCCCGCCCCCCCCCTGAGCAAGGTGCCC |
| (lipocalin Lcn2) | CTGCAGCAGAACTTCCAGGACAACCAGTTCCAGGGCAAGTGGTACGTGGTG |
| | GGCCTGGCCGGCAACGCCATCCTGAGAGAGGACAAGGACCCCCAGAAGATG |
| | TACGCCACCATCTACGAGCTGAAGGAGGACAAGAGCTACAACGTGACCAGC |
| | GTGCTGTTCAGAAAGAAGAAGTGCGACTACTGGATCAGAACCTTCGTGCCC |
| | GGCTGCCAGCCCGGCGAGTTCACCCTGGGCAACATCAAGAGCTACCCCGGC |
| | CTGACCAGCTACCTGGTGAGAGTGGTGAGCACCAACTACAACCAGCACGCC |
| | ATGGTGTTCTTCAAGAAGGTGAGCCAGAACAGAGAGTACTTCAAGATCACC |
| | CTGTACGGCAGAACCAAGGAGCTGACCAGCGAGCTGAAGGAGAACTTCATC |
| | AGATTCAGCAAGAGCCTGGGCCTGCCCGAGAACCACATCGTGTTCCCCGTG |
| | CCCATCGACCAGTGCATCGACGGC (SEQ ID NO: 174) |
| Avimers (C426) | TGCGAGAGCGGCGAGTTCCAGTGCCACAGCACCGGCAGATGCATCCCCCAG |
| targeting c-MET | GAGTGGGTGTGCGACGGCGACAACGACTGCGAGGACAGCAGCGACGAGGCC |
| | CCCGACCTGTGCGCCAGCGCCGAGCCCACCTGCCCCAGCGGCGAGTTCCAG |
| | TGCAGAAGCACCAACAGATGCATCCCCGAGACCTGGCTGTGCGACGGCGAC |
| | AACGACTGCGAGGACGGCAGCGACGAGGAGAGCTGCACCCCCCCCACCTGA |
| | (SEQ ID NO: 176) |
| Centyrins | CTGCCCGCCCCCAAGAACCTGGTGGTGAGCGAGGTGACCGAGGACAGCGCC |
| (Fn3 domain of | AGACTGAGCTGGACCGCCCCCGACGCCGCCTTCGACAGCTTCCTGATCGGC |
| Tenascin) | TACGGCGAGAGCGAGAAGGTGGGCGAGGCCATCGTGCTGACCGTGCCCGGC |
| | AGCGAGAGAAGCTACGACCTGACCGGCCTGAAGCCCGGCACCGAGTACACC |
| | GTGAGCATCTACGGCGTGAAGGGCGGCCACAGAAGCAACCCCCTGAGCGCC |
| | ATCTTCACCACC (SEQ ID NO: 178) |
| Kunitz | GTGAGAGAGGTGTGCAGCGAGCAGGCCGAGACCGGCCCCTGCAGAGCCATG |
| domain/BPTI | ATCAGCAGATGGTACTTCGACGTGACCGAGGGCAAGTGCGCCCCCTTCTTC |
| | TACGGCGGCTGCTGCGGCGGCAACAGAAACAACTTCGACACCGAGGAGTAC |
| | TGCATGGCCGTGTGCGGC (SEQ ID NO: 180) |
| Obodies | GAGATCATGGACGCCGCCGAGGACTACGCCAAGGAGAGATACGGCATCAGC |
| (human AspRS) | AGCATGATCCAGAGCCAGGAGAAGCCCGACAGAGTGCTGGTGAGAGTGAGA |
| | GACCTGACCATCCAGAAGGCCGACGAGGTGGTGTGGGTGAGAGCCAGAGTG |
| | CACACCAGCAGAGCCAAGGGCAAGCAGTGCTTCCTGGTGCTGAGACAGCAG |
| | CAGTTCAACGTGCAGGCCCTGGTGGCCGTGGGCGACCACGCCAGCAAGCAG |
| | ATGGTGAAGTTCGCCGCCAACATCAACAAGGAGAGCATCGTGGACGTGGAG |
| | GGCGTGGTGAGAAAGGTGAACCAGAAGATCGGCAGCTGCACCCAGCAGGAC |
| | GTGGAGCTGCACGTGCAGAAGATCTACGTGATCAGCCTGGCCGAGCCCAGA |
| | CTGCCCCTGCAGCTGGACGACGCCGTGAGACCCGAGGCCGAGGGCGAGGAG |
| | GAGGGCAGAGCCACCGTGAACCAGGACACCAGACTGGACAACAGAGTGATC |
| | GACCTG (SEQ ID NO: 182) |
| Tn3A | GCCATCGAGGTGAAGGACGTGACCGACACCACCGCCCTGATCACCTGGAGC |
| | GACGAGTTCGGCCACGACTACGACGGCTGCGAGCTGACCTACGGCATCAAG |
| | GACGTGCCCGGCGACAGAACCACCATCGACCTGTGGTGGCACAGCGCCTGG |
| | TACAGCATCGGCAACCTGAAGCCCGACACCGAGGACGTGAGCCTGATCTGC |
| | TACACCGACCAGGAGGCCGGCAACCCCGCCAAGGAGACCTTCACCACCGGC |
| | CTGGTGCCCAGA (SEQ ID NO: 184) |
| Tn3B | GCCATCGAGGTGGAGGACGTGACCGACACCACCGCCCTGATCACCTGGACC |
| | AACAGAAGCAGCTACAGCAACCTGCACGGCTGCGAGCTGGCCTACGGCATC |
| | AAGGACGTGCCCGGCGACAGAACCACCATCGACCTGAACCAGCCCTACGTG |
| | CACTACAGCATCGGCAACCTGAAGCCCGACACCGAGTACGAGGTGAGCCTG |
| | ATCTGCCTGACCACCGACGGCACCTACAACAACCCCGCCAAGGAGACCTTC |
| | ACCACCGGCCTGGTGCCCAGA (SEQ ID NO: 186) |
| Hckomers | ACCCTGTTCGTGGCCCTGTACGACTACGAGGCCAGAACCGAGGACGAGCTG |
| | AGCTTCCACAAGGGCGAGAAGTTCCAGATCCTGAACAGCAGCGAGGGCGAC |
| | TGGTGGGAGGCCAGAGACAGCCTGACCACCGGCGAGACCGGCTACATCCCC |
| | AGCAACTACGTGGCCCCCGTGGAC (SEQ ID NO: 188) |
| NPHP1 | GAGGAGTACATCGCCGTGGGCGACTTCGACACCGCCCAGCAGGTGGGCGAC |
| | CTGACCTTCAAGAAGGGCGAGATCCTGCTGGTGATCGAGAAGAAGCCCGAC |
| | GGCTGGTGGATCGCCAAGGACGCCAAGGGCAACGAGGGCCTGGTGCCCAGA |
| | ACCTACCTGGAGCCCTACAGC (SEQ ID NO: 190) |
| Tec | GAGATCGTGGTGGCCATGTACGACTTCCAGGCCGCCGAGGGCCACGACCTG |
| | AGACTGGAGAGACAGGAGTACCTGATCCTGGAGAAGAACGACGTGCACTGG |
| | TGGAGAGCCAGAGACAAGTACGGCAACGAGGGCTACATCCCCAGCAACTAC |
| | GTGACCGGCAAGAAGTGA (SEQ ID NO: 192) |
| Hck | ATCATCGTGGTGGCCCTGTACGACTACGAGGCCATCCACCACGAGGACCTG |
| | AGCTTCCAGAAGGGCGACCAGATGGTGGTGCTGGAGGAGAGCGGCGAGTGG |
| | TGGAAGGCCAGAAGCCTGGCCACCAGAAAGGAGGGCTACATCCCCAGCAAC |
| | TACGTGGCCAGAGTGGAC (SEQ ID NO: 194) |
| Amph | TACAAGGTGGAGACCCTGCACGACTTCGAGGCCGCCAACAGCGACGAGCTG |
| | ACCCTGCAGAGAGGCGACGTGGTGCTGGTGGTGCCCAGCGACAGCGAGGCC |
| | GACCAGGACGCCGGCTGGCTGGTGGGCGTGAAGGAGAGCGACTGGCTGCAG |
| | TACAGAGACCTGGCCACCTACAAGGGCCTGTTCCCCGAGAACTTCACCAGA |
| | AGACTGGAC (SEQ ID NO: 196) |
| RIMBP#3 | AAGATCATGATCGCCGCCCTGGACTACGACCCCGGCGACGGCCAGATGGGC |
| | GGCCAGGGCAAGGGCAGACTGGCCCTGAGAGCCGGCGACGTGGTGATGGTG |
| | TACGGCCCCATGGACGACCAGGGCTTCTACTACGGCGAGCTGGGCGGCCAC |
| | AGAGGCCTGGTGCCCGCCCACCTGCTGGACCACATGAGC (SEQ ID |
| | NO: 198) |
| IRIKS | CAGAAGGTGAAGACCATCTTCCCCCACACCGCCGGCAGCAACAAGACCCTG |
| | CTGAGCTTCGCCCAGGGCGACGTGATCACCCTGCTGATCCCCGAGGAGAAG |
| | GACGGCTGGCTGTACGGCGAGCACGACGTGAGCAAGGCCAGAGGCTGGTTC |
| | CCCAGCAGCTACACCAAGCTGCTGGAG (SEQ ID NO: 200) |
| SNX33 | CTGAAGGGCAGAGCCCTGTACGACTTCCACAGCGAGAACAAGGAGGAGATC |
| | AGCATCCAGCAGGACGAGGACCTGGTGATCTTCAGCGAGACCAGCCTGGAC |
| | GGCTGGCTGCAGGGCCAGAACAGCAGAGGCGAGACCGGCCTGTTCCCCGCC |
| | AGCTACGTGGAGATCGTGAGA (SEQ ID NO: 202) |
| Eps8L1 | AAGTGGGTGCTGTGCAACTACGACTTCCAGGCCAGAAACAGCAGCGAGCTG |
| | AGCGTGAAGCAGAGAGACGTGCTGGAGGTGCTGGACGACAGCAGAAAGTGG |
| | TGGAAGGTGAGAGACCCCGCCGGCCAGGAGGGCTACGTGCCCTACAACATC |
| | CTGACCCCCTACCCC (SEQ ID NO: 204) |
| FISH#5 | GACGTGTACGTGAGCATCGCCGACTACGAGGGCGACGAGGAGACCGCCGGC |
| | TTCCAGGAGGGCGTGAGCATGGAGGTGCTGGAGAGAAACCCCAACGGCTGG |
| | TGGTACTGCCAGATCCTGGACGGCGTGAAGCCCTTCAAGGGCTGGGTGCCC |
| | AGCAACTACCTGGAGAAGAAGAAC (SEQ ID NO: 206) |
| CMS#1 | GTGGACTACATCGTGGAGTACGACTACGACGCCGTGCACGACGACGAGCTG |
| | ACCATCAGAGTGGGCGAGATCATCAGAAACGTGAAGAAGCTGCAGGAGGAG |
| | GGCTGGCTGGAGGGCGAGCTGAACGGCAGAAGAGGCATGTTCCCCGACAAC |
| | TTCGTGAAGGAGATCAAG (SEQ ID NO: 208) |
| OSTF1 | AAGGTGTTCAGAGCCCTGTACACCTTCGAGCCCAGAACCCCCGACGAGCTG |
| | TACTTCGAGGAGGGCGACATCATCTACATCACCGACATGAGCGACACCAAC |
| | TGGTGGAAGGGCACCAGCAAGGGCAGAACCGGCCTGATCCCCAGCAACTAC |
| | GTGGCCGAGCAGGCC (SEQ ID NO: 210) |

TABLE 3
Amino acid and nucleic acid
sequence of glycine-serine linkers

| Amino acid | |
|---|---|
| sequence | Nucleic acid sequence |
| GGGG | GGCGGCGGCGGC (SEQ ID NO: 219) |
| (SEQ ID NO: 220) | |
| GGGS | GGCGGCGGCAGC (SEQ ID NO: 221) |
| (SEQ ID NO: 222) | |
| (GGGS)2 | GGCGGCGGCAGCGGCGGCGGCAGA |
| (SEQ ID NO: 224) | (SEQ ID NO: 223) |
| (GGGS)3 | GGCGGCGGCAGCGGCGGCGGCAGCGGCGG |
| (SEQ ID NO: 226) | CGGCAGA (SEQ ID NO: 225) |
| (GGGS)4 | GGCGGCGGCAGCGGCGGCGGCAGCGGCGG |
| (SEQ ID NO: 228) | CGGCAGAGGCGGCGGCAGA (SEQ ID |
| | NO: 227) |
| (GGGS)5 | GGCGGCGGCAGCGGCGGCGGCAGCGGCGG |
| (SEQ ID NO: 230) | CGGCAGAGGCGGCGGCAGAGGCGGCGGCA |
| | GA (SEQ ID NO: 229) |
| GGGGS | GGCGGCGGCGGCAGC (SEQ ID |
| (SEQ ID NO: 232) | NO: 231) |
| (GGGGS)2 | GGCGGCGGCGGCAGCGGCGGCGGCGGCAG |
| (SEQ ID NO: 234) | C (SEQ ID NO: 233) |
| (GGGGS)3 | GGCGGCGGCGGCAGCGGCGGCGGCGGCAG |
| (SEQ ID NO: 236) | CGGCGGCGGCGGCAGC (SEQ ID |
| | NO: 235) |
| GGGSGGGGSGGGS | GGCGGCGGCAGCGGCGGCGGCGGCAGCGG |
| (SEQ ID NO: 238) | CGGCAGC (SEQ ID NO: 237) |
| GGSG | GGCGGCAGCGGC (SEQ ID NO: 239) |
| (SEQ ID NO: 240) | |
| (GGSG)2 | GGCGGCAGCGGCGGCGGCAGCGGC (SEQ |
| (SEQ ID NO: 242) | ID NO: 241) |
| (GGSG)3 | GGCGGCAGCGGCGGCGGCAGCGGCGGCGG |
| (SEQ ID NO: 244) | CAGCGGC (SEQ ID NO: 243) |
| SGGGGIG | AGCGGCGGCGGCGGCATCGGC (SEQ ID |
| (SEQ ID NO: 246) | NO: 245) |
| SGGGGSGGGGIG | AGCGGCGGCGGCGGCAGCGGCGGCGGCGG |
| (SEQ ID NO: 248) | CATCGGC (SEQ ID NO: 247) |
| SGGGG | AGCGGCGGCGGCGGC (SEQ ID |
| (SEQ ID NO: 250) | NO: 249) |

TABLE 4
Amino acid sequence of
human IgG Fc and and IgM Cμ4tp

| Human IgG | Sequence (SEQ ID NO:) |
|---|---|
| IgG1 | DKTHTCPPCPAPELLGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYKCKVSN |
| | KALPAPIEKTISKAKGQPREPQVYTLPPSREEMTK |
| | NQVSLTCLVKGFYPSDIAVEWESNGQPENNYKTTP |
| | PVLDSDGSFFLYSKLTVDKSRWQQGNVFSCSVMHE |
| | ALHNHYTQKSLSLSPGK (SEQ ID NO: 252) |
| IgG2 | VECPPCPAPPVAGPSVFLFPPKPKDTLMISRTPEV |
| | TCVVVDVSHEDPEVQFNWYVDGMEVHNAKTKPREE |
| | QFNSTFRVVSVLTVVHQDWLNGKEYKCKVSNKGLP |
| | APIEKTISKTKGQPREPQVYTLPPSREEMTKNQVS |
| | LTCLVKGFYPSDIAVEWESNGQPENNYKTTPPMLD |
| | SDGSFFLYSKLTVDKSRWQQGNVFSCSVMHEALHN |
| | HYTQKSLSLSPGK (SEQ ID NO: 254) |
| IgG3 | DTPPPCPRCPAPELLGGPSVFLFPPKPKDTLMISR |
| | TPEVTCVVVDVSHEDPEVQFKWYVDGVEVHNAKTK |
| | PREEQYNSTFRVVSVLTVLHQDWLNGKEYKCKVSN |
| | KALPAPIEKTISKTKGQPREPQVYTLPPSREEMTK |
| | NQVSLTCLVKGFYPSDIAVEWESSGQPENNYNTTP |
| | PMLDSDGSFFLYSKLTVDKSRWQQGNIFSCSVMHE |
| | ALHNRFTQKSLSLSPGK (SEQ ID NO: 256) |
| IgG4 | PPCPSCPAPEFLGGPSVFLFPPKPKDTLMISRTPE |
| | VTCVVVDVSQEDPEVQFNWYVDGVEVHNAKTKPRE |
| | EQFNSTYRVVSVLTVLHQDWLNGKEYKCKVSNKGL |
| | PSSIEKTISKAKGQPREPQVYTLPPSQEEMTKNQV |
| | SLTCLVKGFYPSDIAVEWESNGQPENNYKTTPPVL |
| | DSDGSFFLYSRLTVDKSRWQEGNVFSCSVMHEALH |
| | NHYTQKSLSLSPGK (SEQ ID NO: 258) |
| Human IgM | KHPPAVYLLPPAREQLNLRESATVTCLVKGFSPAD |
| Cμ4tp | ISVQWLQRGQLLPQEKYVTSAPMPEPGAPGFYFTH |
| | SILTVTEEEWNSGETYTCVVGHEALPHLVTERTVD |
| | KSTGKPTLYNVSLIMSDTGGTCY (SEQ ID |
| | NO: 260) |
| Human IgA | TFPPQVHLLPPPSEELALNELLSLTCLVRAFNPKE |
| Cα3tp | VLVRWLHGNEELSPESYLVFEPLKEPGEGATTYLV |
| | TSVLRVSAETWKQGDQYSCMVGHEALPMNFTQKTI |
| | DRLSGKPTNVSVSVIMSEGDGICY (SEQ ID |
| | NO: 262) |

TABLE 5
Nucleic acid sequence of human IgG Fc and and IgM Cμ4tp

| Human IgG | Sequence (SEQ ID NO:) |
|---|---|
| IgG1 | GACAAAACTCACACATGCCCACCGTGCCCAGCACCTGAACTCCTGGGGGGAC |
| | CGTCAGTCTTCCTCTTCCCCCCAAAACCCAAGGACACCCTCATGATCTCCCG |
| | GACCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGACAA |
| | AGCCGCGGGAGGAGCAGTACAACAGCACGTACCGTGTGGTCAGCGTCCTCAC |
| | CGTCCTGCACCAGGACTGGCTGAATGGCAAGGAGTACAAGTGCAAGGTCTCC |
| | AACAAAGCCCTCCCAGCCCCCATCGAGAAAACCATCTCCAAAGCCAAAGGGC |
| | AGCCCCGAGAACCACAGGTGTACACCCTGCCCCCATCCCGGGAGGAGATGAC |
| | CAAGAACCAGGTCAGCCTGACCTGCCTGGTCAAAGGCTTCTATCCCAGCGAC |
| | ATCGCCGTGGAGTGGGAGAGCAATGGGCAGCCGGAGAACAACTACAAGACCA |
| | CGCCTCCCGTGCTGGACTCCGACGGCTCCTTCTTCCTCTACAGCAAGCTCAC |
| | CGTGGACAAGAGCAGGTGGCAGCAGGGGAACGTCTTCTCATGCTCCGTGATG |
| | CACGAGGCTCTGCACAACCACTACACGCAGAAGAGCCTCTCCCTGTCTCCGG |
| | GTAAA (SEQ ID NO: 251) |
| IgG2 | GTGGAGTGCCCACCTTGCCCAGCACCACCTGTGGCAGGACCTTCAGTCTTCC |
| | TCTTCCCCCCAAAACCCAAGGACACCCTGATGATCTCCAGAACCCCTGAGGT |
| | CACGTGCGTGGTGGTGGACGTGAGCCACGAAGACCCCGAGGTCCAGTTCAAC |
| | TGGTACGTGGACGGCATGGAGGTGCATAATGCCAAGACAAAGCCACGGGAGG |
| | AGCAGTTCAACAGCACGTTCCGTGTGGTCAGCGTCCTCACCGTCGTGCACCA |
| | GGACTGGCTGAACGGCAAGGAGTACAAGTGCAAGGTCTCCAACAAAGGCCTC |
| | CCAGCCCCCATCGAGAAAACCATCTCCAAAACCAAAGGGCAGCCCCGAGAAC |
| | CACAGGTGTACACCCTGCCCCCATCCCGGGAGGAGATGACCAAGAACCAGGT |
| | CAGCCTGACCTGCCTGGTCAAAGGCTTCTACCCCAGCGACATCGCCGTGGAG |
| | TGGGAGAGCAATGGGCAGCCGGAGAACAACTACAAGACCACACCTCCCATGC |
| | TGGACTCCGACGGCTCCTTCTTCCTCTACAGCAAGCTCACCGTGGACAAGAG |
| | CAGGTGGCAGCAGGGGAACGTCTTCTCATGCTCCGTGATGCATGAGGCTCTG |
| | CACAACCACTACACACAGAAGAGCCTCTCCCTGTCTCCGGGTAAA (SEQ |
| | ID NO: 253) |
| IgG3 | GACACACCTCCCCCGTGCCCAAGGTGCCCAGCACCTGAACTCCTGGGAGGAC |
| | CGTCAGTCTTCCTCTTCCCCCCAAAACCCAAGGATACCCTTATGATTTCCCG |
| | GACCCCTGAGGTCACGTGCGTGGTGGTGGACGTGAGCCACGAAGACCCCGAG |
| | GTCCAGTTCAAGTGGTACGTGGACGGCGTGGAGGTGCATAATGCCAAGACAA |
| | AGCCGCGGGAGGAGCAGTACAACAGCACGTTCCGTGTGGTCAGCGTCCTCAC |
| | CGTCCTGCACCAGGACTGGCTGAACGGCAAGGAGTACAAGTGCAAGGTCTCC |
| | AACAAAGCCCTCCCAGCCCCCATCGAGAAAACCATCTCCAAAACCAAAGGAC |
| | AGCCCCGAGAACCACAGGTGTACACCCTGCCCCCATCCCGGGAGGAGATGAC |
| | CAAGAACCAGGTCAGCCTGACCTGCCTGGTCAAAGGCTTCTACCCCAGCGAC |
| | ATCGCCGTGGAGTGGGAGAGCAGCGGGCAGCCGGAGAACAACTACAACACCA |
| | CGCCTCCCATGCTGGACTCCGACGGCTCCTTCTTCCTCTACAGCAAGCTCAC |
| | CGTGGACAAGAGCAGGTGGCAGCAGGGGAACATCTTCTCATGCTCCGTGATG |
| | CATGAGGCTCTGCACAACCGCTTCACGCAGAAGAGCCTCTCCCTGTCTCCGG |
| | GTAAA (SEQ ID NO: 255) |
| IgG4 | CCCCCATGCCCATCATGCCCAGCACCTGAGTTCCTGGGGGGACCATCAGTCT |
| | TCCTGTTCCCCCCAAAACCCAAGGACACTCTCATGATCTCCCGGACCCCTGA |
| | GGTCACGTGCGTGGTGGTGGACGTGAGCCAGGAAGACCCCGAGGTCCAGTTC |
| | AACTGGTACGTGGATGGCGTGGAGGTGCATAATGCCAAGACAAAGCCGCGGG |
| | AGGAGCAGTTCAACAGCACGTACCGTGTGGTCAGCGTCCTCACCGTCCTGCA |
| | CCAGGACTGGCTGAACGGCAAGGAGTACAAGTGCAAGGTCTCCAACAAAGGC |
| | CTCCCGTCCTCCATCGAGAAAACCATCTCCAAAGCCAAAGGGCAGCCCCGAG |
| | AGCCACAGGTGTACACCCTGCCCCCATCCCAGGAGGAGATGACCAAGAACCA |
| | GGTCAGCCTGACCTGCCTGGTCAAAGGCTTCTACCCCAGCGACATCGCCGTG |
| | GAGTGGGAGAGCAATGGGCAGCCGGAGAACAACTACAAGACCACGCCTCCCG |
| | TGCTGGACTCCGACGGCTCCTTCTTCCTCTACAGCAGGCTAACCGTGGACAA |
| | GAGCAGGTGGCAGGAGGGGAATGTCTTCTCATGCTCCGTGATGCATGAGGCT |
| | CTGCACAACCACTACACACAGAAGAGCCTCTCCCTGTCTCCGGGTAAA |
| | (SEQ ID NO: 257) |
| Human IgM | AAGCACCCCCCCGCCGTGTACCTGCTGCCCCCCGCCAGAGAGCAGCTGAACC |
| Cμ4tp | TGAGAGAGAGCGCCACCGTGACCTGCCTGGTGAAGGGCTTCAGCCCCGCCGA |
| | CATCAGCGTGCAGTGGCTGCAGAGAGGCCAGCTGCTGCCCCAGGAGAAGTAC |
| | GTGACCAGCGCCCCCATGCCCGAGCCCGGCGCCCCCGGCTTCTACTTCACCC |
| | ACAGCATCCTGACCGTGACCGAGGAGGAGTGGAACAGCGGCGAGACCTACAC |
| | CTGCGTGGTGGGCCACGAGGCCCTGCCCCACCTGGTGACCGAGAGAACCGTG |
| | GACAAGAGCACCGGCAAGCCCACCCTGTACAACGTGAGCCTGATCATGAGCG |
| | ACACCGGCGGCACCTGCTACTGA (SEQ ID NO: 259) |
| Human IgA | ACCTTCCCCCCCCAGGTGCACCTGCTGCCCCCCCCCAGCGAGGAGCTGGCCC |
| Cα3tp | TGAACGAGCTGCTGAGCCTGACCTGCCTGGTGAGAGCCTTCAACCCCAAGGA |
| | GGTGCTGGTGAGATGGCTGCACGGCAACGAGGAGCTGAGCCCCGAGAGCTAC |
| | CTGGTGTTCGAGCCCCTGAAGGAGCCCGGCGAGGGCGCCACCACCTACCTGG |
| | TGACCAGCGTGCTGAGAGTGAGCGCCGAGACCTGGAAGCAGGGCGACCAGTA |
| | CAGCTGCATGGTGGGCCACGAGGCCCTGCCCATGAACTTCACCCAGAAGACC |
| | ATCGACAGACTGAGCGGCAAGCCCACCAACGTGAGCGTGAGCGTGATCATGA |
| | GCGAGGGCGACGGCATCTGCTACTGA (SEQ ID NO: 261) |

TABLE 6
Amino acid sequence of multimerization domain.

| | |
|---|---|
| Type I | VADFLIIYIEEAHATDGWAL (SEQ ID |
| deiodinase | NO: 264) |
| dimerization | |
| motif | |
| Trimerization | |
| motifs | |
| GCN4 | IKQIEDKIEEILSKIYHIENEIARIKKL (SEQ |
| | ID NO: 266) |
| Matrilin 1 | CACESLVKFQAKVEGLLQALTRKLEAVSKRLAILE |
| | NTVV (SEQ ID NO: 268) |
| Coronin 1a | VSRLEEEMRKLQATVQELQKRLDRLEETVQAK |
| | (SEQ ID NO: 270) |
| CMP | ESLVKFQAKVEGLLQALTRKLEAVSKRLAILENTV |
| | V (SEQ ID NO: 272) |
| DMPK | EAEAEVTLRELQEALEEEVLTRQSLSREMEAIRTD |
| | NQNFASQLREAEARNRDLEAHVRQLQERMELLQAE |
| | (SEQ ID NO: 274) |
| Langerin | ASALNTKIRALQGSLENMSKLLKRQNDILQVVS |
| | (SEQ ID NO: 276) |
| Surfectin | DVASLRQQVEALQGQVQHLQAAFSQYKKV (SEQ |
| Protein SP-D | ID NO: 278) |
| Tenascin-C | ACGCAAAPDVKELLSRLEELENLVSSLREQ (SEQ |
| | ID NO: 280) |
| Tenascin-R | ACPCASSAQVLQELLSRIEMLEREVSVLRDQ |
| | (SEQ ID NO: 282) |
| Tenascin-X | GCGCPPGTEPPVLASEVQALRVRLEILEELVKGLK |
| | EQ (SEQ ID NO: 284) |
| Tetrameric | ESLVKFQAKVEGLLQALTRKLEAVSKQLAILENTV |
| motif | V (SEQ ID NO: 286) |
| CMP (R27Q) | |
| Pentameric | DLAPQMLRELQETNAALQDVRELLRQQVKEITFLK |
| motif (COMP) | NTVMECDACG (SEQ ID NO: 288) |

TABLE 7
Nucleic acid sequence of multimerization domain.

| | |
|---|---|
| Dimerization | GTGGCCGACTTCCTGATCATCTACATCGAGGAG |
| motif | GCCCACGCCACCGACGGCTGGGCCCTG (SEQ |
| | ID NO: 263) |
| Trimerization | |
| motifs | |
| GCN4 | ATCAAGCAGATCGAGGACAAGATCGAGGAGATC |
| | CTGAGCAAGATCTACCACATCGAGAACGAGATC |
| | GCCAGAATCAAGAAGCTG (SEQ ID |
| | NO: 265) |
| Matrilin 1 | TGCGCCTGCGAGAGCCTGGTGAAGTTCCAGGCC |
| | AAGGTGGAGGGCCTGCTGCAGGCCCTGACCAGA |
| | AAGCTGGAGGCCGTGAGCAAGAGACTGGCCATC |
| | CTGGAGAACACCGTGGTG (SEQ ID |
| | NO: 267) |
| Coronin 1a | GTGAGCAGACTGGAGGAGGAGATGAGAAAGCTG |
| | CAGGCCACCGTGCAGGAGCTGCAGAAGAGACTG |
| | GACAGACTGGAGGAGACCGTGCAGGCCAAG |
| | (SEQ ID NO: 269) |
| CMP | GAGAGCCTGGTGAAGTTCCAGGCCAAGGTGGAG |
| | GGCCTGCTGCAGGCCCTGACCAGAAAGCTGGAG |
| | GCCGTGAGCAAGAGACTGGCCATCCTGGAGAAC |
| | ACCGTGGTG (SEQ ID NO: 271) |
| DMPK | GAGGCCGAGGCCGAGGTGACCCTGAGAGAGCTG |
| | CAGGAGGCCCTGGAGGAGGAGGTGCTGACCAGA |
| | CAGAGCCTGAGCAGAGAGATGGAGGCCATCAGA |
| | ACCGACAACCAGAACTTCGCCAGCCAGCTGAGA |
| | GAGGCCGAGGCCAGAAACAGAGACCTGGAGGCC |
| | CACGTGAGACAGCTGCAGGAGAGAATGGAGCTG |
| | CTGCAGGCCGAG (SEQ ID NO: 273) |
| Langerin | GCCAGCGCCCTGAACACCAAGATCAGAGCCCTG |
| | CAGGGCAGCCTGGAGAACATGAGCAAGCTGCTG |
| | AAGAGACAGAACGACATCCTGCAGGTGGTGAGC |
| | (SEQ ID NO: 275) |
| Surfectin | GACGTGGCCAGCCTGAGACAGCAGGTGGAGGCC |
| Protein SP-D | CTGCAGGGCCAGGTGCAGCACCTGCAGGCCGCC |
| | TTCAGCCAGTACAAGAAGGTG (SEQ ID |
| | NO: 277) |
| Tenascin-C | GCCTGCGGCTGCGCCGCCGCCCCCGACGTGAAG |
| | GAGCTGCTGAGCAGACTGGAGGAGCTGGAGAAC |
| | CTGGTGAGCAGCCTGAGAGAGCAG (SEQ ID |
| | NO: 279) |
| Tenascin-R | GCCTGCCCCTGCGCCAGCAGCGCCCAGGTGCTG |
| | CAGGAGCTGCTGAGCAGAATCGAGATGCTGGAG |
| | AGAGAGGTGAGCGTGCTGAGAGACCAG (SEQ |
| | ID NO: 281) |
| Tenascin-X | GGCTGCGGCTGCCCCCCCGGCACCGAGCCCCCC |
| | GTGCTGGCCAGCGAGGTGCAGGCCCTGAGAGTG |
| | AGACTGGAGATCCTGGAGGAGCTGGTGAAGGGC |
| | CTGAAGGAGCAG (SEQ ID NO: 283) |
| Tetrameric | GAGAGCCTGGTGAAGTTCCAGGCCAAGGTGGAG |
| motif | GGCCTGCTGCAGGCCCTGACCAGAAAGCTGGAG |
| CMP (R27Q) | GCCGTGAGCAAGCAGCTGGCCATCCTGGAGAAC |
| | ACCGTGGTG (SEQ ID NO: 285) |
| Pentameric | GACCTGGCCCCCCAGATGCTGAGAGAGCTGCAG |
| motif | GAGACCAACGCCGCCCTGCAGGACGTGAGAGAG |
| (COMP) | CTGCTGAGACAGCAGGTGAAGGAGATCACCTTC |
| | CTGAAGAACACCGTGATGGAGTGCGACGCCTGC |
| | GGC (SEQ ID NO: 287) |

The RRVs of the disclosure can be engineered to modify their stability and/or expression. For example, changes in expression can occur due to the frequency with which inactivating or attenuating mutations accumulate in the replicating retroviral vector as it progressively replicates in tumor tissue. Investigation shows that one of the most frequent events is G to A mutations (corresponds to the C to T characteristic ApoBec mediated mutations in the negative strand single stranded DNA from the first replicative step in the reverse transcription step). This can cause changes in amino acid composition of the RRV proteins and a devastating change from TGG (Tryptophan) to stop codons (TAG or TGA). In one embodiment this inactivating change is avoided by engineering codons of other amino acids with similar chemical or structural properties such as phenylalanine or tyrosine in place of a tryptophan codon.

Thus, in addition to the 2A-peptide-SSP cassette the RRV can include a plurality of additional mutations that improve expression and/or stability of the construct in a host cell. Such mutations can include modifications of one or more codons in the GAG, POL and/or ENV coding sequences that change a tryptophan codon to a permissible codon that maintains the biological activity of the GAG, POL and/or ENV domains. It is known in the art that the codon for tryptophan is UGG (TGG in DNA). Moreover, it is known in the art that the “stop codon” is UAA, UAG or UGA (TAA, TAG or TGA in DNA). A single point mutation in the tryptophan codon can cause an unnatural stop codon (e.g., UGG->UAG or UGG->UGA). It is also known that human APOBEC3GF (hA3G/F) inhibits retroviral replication through G->A hypermutations (Neogi et al., J. Int. AIDS Soc., 16(1):18472, Feb. 25, 2013). Moreover, as described below long term expression and viral stability can be improved by avoiding use of tryptophan codons in coding sequence, thereby avoiding the incorporation of unnatural stop codons due to hypermutation cause by hA3G/F. For example, in one embodiment, an MLV derived nucleic acid sequence comprises GAG, POL and ENV coding domains can comprise modification of codons containing the nucleotides identified in Table A (nucleotide number referenced to SEQ ID NO:2), which are in tryptophan codons, one can provide hA3C/F resistant RRVs.

TABLE A
Summary of recurrent G to A mutations that lead to tryptophan
to stop codon changes. Nucleotide is the position in SEQ ID
NO: 2 RRV genome, “gene” is the gene the nucleotide
is located in and AA is the amino acid position in the polypeptide.

| nucleotide | gene | AA |
|---|---|---|
| 1306 | GAG | 35 |
| 5299 | POL | 718 |
| 5557 | POL | 804 |
| 5806 | POL | 887 |
| 6193 | POL | 1016 |
| 6232 | POL | 1029 |
| 6298 | POL | 1051 |
| 6801 | ENV | 148 |
| 6978 | ENV | 207 |
| 7578 | ENV | 407 |

Thus, in one embodiment of the disclosure, a recombinant replication competent retrovirus is provided that comprises one or more mutations in codons for tryptophan, wherein the mutation changes the codon to a codon for an amino acid other than tryptophan and that provide codons that are biocompatible (i.e., codons that do not disrupt the function of the vector). This vector is sometimes referred to herein as an “ApoBec inactivation resistant vector” or “ApoBec resistant vector”. The recombinant ApoBec inactivation resistant vector can comprise an IRES cassette, promoter cassette and/or 2A peptide-SSP cassette.

As mentioned above, human APOBEC3g causes hypermutations in viral vector sequences converting G->A (Hogan et al., Can. Res., 2018). Accordingly, tryptophan codons in heterologous polynucleotides contained in the 2A-SSP peptide cassette are susceptible to being converted by hAPOBEC3 to stop codons. To avoid such mutations, tryptophan codons can be replaced with biologically permissible codons for other amino acids. For example, in one embodiment, a 2A-SSP cassette of the disclosure can comprise a polynucleotide encoding a polypeptide having cytosine deaminase activity, wherein the polynucleotide comprises the sequence:

(SEQ ID NO: 28)
atg gtg acc ggc ggc atg gcc tcc aag tgg gat caa
aag ggc atg gat atc gct tac gag gag gcc ctg ctg
ggc tac aag gag ggc ggc gtg cct atc ggc ggc tgt
ctg atc aac aac aag gac ggc agt gtg ctg ggc agg
ggc cac aac atg agg ttc cag aag ggc tcc gcc acc
ctg cac ggc gag atc tcc acc ctg gag aac tgt ggc
agg ctg gag ggc aag gtg tac aag gac acc acc ctg
tac acc acc ctg tcc cct tgt gac atg tgt acc ggc
gct atc atc atg tac ggc atc cct agg tgt gtg atc
ggc gag aac gtg aac ttc aag tcc aag ggc gag aag
tac ctg caa acc agg ggc cac gag gtg gtg gtt gtt
gac gat gag agg tgt aag aag ctg atg aag cag ttc
atc gac gag agg cct cag gac tgg ttc gag gat atc
ggc gag taa

(or the foregoing wherein “t” is “u”).

This sequence comprises two tryptophan codons (bold/underlined). In one embodiment of the disclosure these codons are independently changed to a codon providing an amino acid selected from the group consisting of D, M, T, E, S, Q, N, F, Y, A, K, H, P, R, V, L, G, I and C. The resulting polypeptide comprises a sequence:

(SEQ ID NO: 29)
M V T G G M A S K X D Q K G M D I A Y E E A L L
G Y K E G G V P I G G C L I N N K D G S V L G R
G H N M R F Q K G S A T L H G E I S T L E N C G
R L E G K V Y K D T T L Y T T L S P C D M C T G
A I I M Y G I P R C V I G E N V N F K S K G E K
Y L Q T R G H E V V V V D D E R C K K L M K Q F
I D E R P Q D X F E D I G E,

wherein the polypeptide comprises cytosine deaminase activity, wherein X is any amino acid except tryptophan. In one embodiment, X in SEQ ID NO:29 are each independently selected from the group consisting of F, D, M, L, S or R.

In another embodiment, a replication competent retroviral vector can comprise a heterologous polynucleotide encoding a polypeptide comprising a cytosine deaminase (as described herein) and may further comprise a polynucleotide comprising a miRNA or siRNA molecule either as part of the primary transcript from the viral promoter or linked to a promoter, which can be cell-type or tissue specific. In yet a further embodiment, the miRNA or siRNA may be preceded by a pol III promoter.

MicroRNAs (miRNA) are small, non-coding RNAs. They are located within introns of coding or non-coding genes, exons of non-coding genes or in inter-genic regions. miRNA coding sequences are transcribed by RNA polymerase III that generate precursor polynucleotides called primary precursor miRNA (pri-miRNA). The pri-miRNA in the nucleus is processed by the ribonuclease Drosha to produce the miRNA precursor (pre-miRNA) that forms a short hairpin structure. Subsequently, pre-miRNA is transported to the cytoplasm via Exportin 5 and further processed by another ribonuclease called Dicer to generate an active, mature miRNA. An siRNA sequence is not preceded by an SSP coding sequence, rather the siRNA is part of a second cassette present in a therapeutic cassette in the viral vector.

A mature miRNA is approximately 21 nucleotides in length. It exerts in function by binding to the 3′ untranslated region of mRNA of targeted genes and suppressing protein expression either by repression of protein translation or degradation of mRNA. miRNA are involved in biological processes including development, cell proliferation, differentiation and cancer progression. Studies of miRNA profiling indicate that some miRNA expressions are tissue specific or enriched in certain tissues. For example, miR-142-3p, miR-181 and miR-223 expressions have demonstrated to be enriched in hematopoietic tissues in human and mouse (Baskerville et al., 2005 RNA 11, 241-247; Chen et al., 2004 Science 303, 83-86).

Some miRNAs have been observed to be up-regulated (oncogenic miRNA) or down-regulated (repressor) in several tumors (Spizzo et al., 2009 Cell 137, 586e1). For example, miR-21 is overexpressed in glioblastoma, breast, lung, prostate, colon, stomach, esophageal, and cervical cancer, uterine leiomyosarcoma, DLBCL, head and neck cancer. In contrast, members of let-7 have reported to be down-regulated in glioblastoma, lung, breast, gastric, ovary, prostate and colon cancers. Re-establishment of homeostasis of miRNA expression in cancer is an imperative mechanism to inhibit or reverse cancer progression.

miRNAs that are down-regulated in cancers could be useful as anticancer agents. Examples include mir-128-1, let-7, miR-26, miR-124, and miR-137 (Esquela-Kerscher et al., 2008 Cell Cycle 7, 759-764; Kumar et al., 2008 Proc Natl Acad Sci USA 105, 3903-3908; Kota et al., 2009 Cell 137, 1005-1017; Silber et al., 2008 BMC Medicine 6:14 1-17). miR-128 expression has reported to be enriched in the central nervous system and has been observed to be down-regulated in glioblastomas (Sempere et al., 2004 Genome Biology 5:R13.5-11; Godlewski et al., 2008 Cancer Res 68: (22) 9125-9130). miR-128 is encoded by two distinct genes, miR-128-1 and miR-128-2. Both are processed into identical mature sequence. Bmi-1 and E2F3a have been reported to be the direct targets of miR-128 (Godlewski et al., 2008 Cancer Res 68: (22) 9125-9130; Zhang et al., 2009 J. Mol Med 87:43-51). In addition, Bmi-1 expression has been observed to be up-regulated in a variety of human cancers, including gliomas, mantle cell lymphomas, non-small cell lung cancer B-cell non-Hodgkin's lymphoma, breast, colorectal and prostate cancer. Furthermore, Bmi-1 has been demonstrated to be required for the self-renewal of stem cells from diverse tissues, including neuronal stem cells as well as “stem-like” cell population in gliomas.

Suitable range for designing stem lengths of a hairpin duplex, includes stem lengths of 20-30 nucleotides, 30-50 nucleotides, 50-100 nucleotides, 100-150 nucleotides, 150-200 nucleotides, 200-300 nucleotides, 300-400 nucleotides, 400-500 nucleotides, 500-600 nucleotides, and 600-700 nucleotides. Suitable range for designing loop lengths of a hairpin duplex, includes loop lengths of 4-25 nucleotides, 25-50 nucleotides, or longer if the stem length of the hair duplex is substantial. In certain context, hairpin structures with duplexed regions that are longer than 21 nucleotides may promote effective siRNA-directed silencing, regardless of the loop sequence and length.

In yet another or further embodiment, the heterologous polynucleotide can comprise a cytokine such as an interleukin, interferon gamma or the like. Cytokines that may expressed from a retroviral vector of the disclosure include, but are not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, anti-CD40, CD40L, IFN-gamma and TNF-alpha, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, 4-1BBL, DcR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TRANK, TR9 (International Publication No. WO 98/56892), TR10 (International Publication No. WO 98/54202), 312C2 (International Publication No. WO 98/06842), and TR12, and soluble forms CD154, CD70, and CD153. Angiogenic proteins may be useful in some embodiments, particularly for protein production from cell lines. Such angiogenic factors include, but are not limited to, Glioma Derived Growth Factor (GDGF), Platelet Derived Growth Factor-A (PDGF-A), Platelet Derived Growth Factor-B (PDGF-B), Placental Growth Factor (PIGF), Placental Growth Factor-2 (PIGF-2), Vascular Endothelial Growth Factor (VEGF), Vascular Endothelial Growth Factor-A (VEGF-A), Vascular Endothelial Growth Factor-2 (VEGF-2), Vascular Endothelial Growth Factor B (VEGF-3), Vascular Endothelial Growth Factor B-1 86 (VEGF-B186), Vascular Endothelial Growth Factor-D (VEGF-D), Vascular Endothelial Growth Factor-D (VEGF-D), and Vascular Endothelial Growth Factor-E (VEGF-E). Fibroblast Growth Factors may be delivered by a vector of the disclosure and include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15. Hematopoietic growth factors may be delivered using vectors of the disclosure, such growth factors include, but are not limited to, granulocyte macrophage colony stimulating factor (GM-CSF) (sargramostim), granulocyte colony stimulating factor (G-CSF) (filgrastim), macrophage colony stimulating factor (M-CSF, CSF-1) erythropoietin (epoetin alfa), stem cell factor (SCF, c-kit ligand, steel factor), megakaryocyte colony stimulating factor, PIXY321 (a GMCSF/IL-3) fusion protein and the like.

The heterologous nucleic acid sequence is typically under control of the viral LTR promoter-enhancer elements. Accordingly, the recombinant retroviral vectors of the disclosure, the desired sequences, genes and/or gene fragments can be inserted at several sites and under different regulatory sequences. For example, a site for insertion can be the viral enhancer/promoter proximal site (i.e., 5′ LTR-driven gene locus).

In one embodiment, the retroviral genome of the disclosure contains a 2A peptide or 2A peptide-like coding sequence upstream of an SSP coding sequence, wherein the SSP coding sequence is followed by a cloning site downstream for insertion of a desired/heterologous polynucleotide. In one embodiment, the 2A peptide or 2A peptide-like coding sequence is located 3′ to the env gene in the retroviral vector, but 5′ to the SSP coding sequence and the desired heterologous polynucleotide. Accordingly, a heterologous polynucleotide encoding a desired polypeptide is operably linked to the 2A peptide or 2A peptide-like-SSP coding sequences.

In another embodiment, a targeting polynucleotide sequence is included as part of the recombinant retroviral vector of the disclosure. The targeting polynucleotide sequence is a targeting ligand (e.g., peptide hormones such as heregulin, a single-chain antibodies, a receptor or a ligand for a receptor), a tissue-specific or cell-type specific regulatory element (e.g., a tissue-specific or cell-type specific promoter or enhancer), or a combination of a targeting ligand and a tissue-specific/cell-type specific regulatory element. Preferably, the targeting ligand is operably linked to or present in the env protein of the retrovirus, creating a chimeric retroviral env protein. The viral GAG, viral POL and viral ENV proteins can be derived from any suitable retrovirus (e.g., MLV or lentivirus-derived). In another embodiment, the viral ENV protein is non-retrovirus-derived (e.g., CMV or VSV).

In one embodiment, the recombinant retrovirus of the disclosure is genetically modified in such a way that the virus is targeted to a particular cell type (e.g., smooth muscle cells, hepatic cells, renal cells, fibroblasts, keratinocytes, mesenchymal stem cells, bone marrow cells, chondrocyte, epithelial cells, intestinal cells, mammary cells, neoplastic cells, glioma cells, neuronal cells and others known in the art) such that the recombinant genome of the retroviral vector is delivered to a target non-dividing, a target dividing cell, or a target cell having a cell proliferative disorder.

In one embodiment, the disclosure provides a recombinant retrovirus capable of infecting a non-dividing cell, a dividing cell, or a cell having a cell proliferative disorder. The recombinant replication competent retrovirus of the disclosure comprises a polynucleotide sequence encoding a viral GAG, a viral POL, a viral ENV, a 2A peptide or 2A peptide-like coding sequence immediately downstream (e.g., between 1 to 50 nucleotides downstream (1-10, 10-15, 15-20, 20-25, 25-30, 30-35, 35-40, 40-45, 45-50 or any integer therebetween) of the viral ENV sequence and an SSP coding sequence operably linked to a heterologous gene and encapsulated within a virion.

The phrase “non-dividing” cell refers to a cell that does not go through mitosis. Non-dividing cells may be blocked at any point in the cell cycle, (e.g., G₀/G₁, Gus, G₂/M), so long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., terminally differentiated cells). For example, a recombinant lentivirus vector is capable of infecting non-dividing cells. Examples of pre-existing non-dividing cells in the body include neuronal, muscle, liver, skin, heart, lung, and bone marrow cells, and their derivatives. For dividing cells gammaretroviral vectors can be used as this type of retrovirus only productively infects dividing cells and this property contributes to the tumor selectivity of this vector class.

By “dividing” cell is meant a cell that undergoes active mitosis, or meiosis. Such dividing cells include stem cells, skin cells (e.g., fibroblasts and keratinocytes), endothelial cells, gametes, and other dividing cells known in the art. Of particular interest and encompassed by the term dividing cell are cells having cell proliferative disorders, such as neoplastic cells. The term “cell proliferative disorder” refers to a condition characterized by an abnormal number of cell divisions. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well. Cell proliferative disorders include disorders associated with an overgrowth of connective tissues, such as various fibrotic conditions, including scleroderma, arthritis and liver cirrhosis. Cell proliferative disorders include neoplastic disorders such as head and neck carcinomas. Head and neck carcinomas would include, for example, carcinoma of the mouth, esophagus, throat, larynx, thyroid gland, tongue, lips, salivary glands, nose, paranasal sinuses, nasopharynx, superior nasal vault and sinus tumors, esthesioneuroblastoma, squamous cell cancer, malignant melanoma, sinonasal undifferentiated carcinoma (SNUC), brain (including glioblastomas such as glioblastoma multiforme) or blood neoplasia. Also included are carcinoma's of the regional lymph nodes including cervical lymph nodes, prelaryngeal lymph nodes, pulmonary juxtaesophageal lymph nodes and submandibular lymph nodes (Harrison's Principles of Internal Medicine (eds., Isselbacher, et al., McGraw-Hill, Inc., 13th Edition, pp 1850-1853, 1994). Other cancer types, include, but are not limited to, lung cancer, colon-rectum cancer, breast cancer, prostate cancer, urinary tract cancer, uterine cancer lymphoma, oral cancer, pancreatic cancer, leukemia, melanoma, stomach cancer, skin cancer and ovarian cancer. The cell proliferative disease also includes rheumatoid arthritis (O'Dell NEJM 350:2591 2004) and other auto-immune disorders (Mackay et al NEJM 345:340 2001) that are often characterized by inappropriate proliferation of cells of the immune system.

In one embodiment, the retroviral vector is targeted to the cell by binding to cells having a molecule on the external surface of the cell. This method of targeting the retrovirus utilizes expression of a targeting ligand on the surface of the retrovirus to assist in targeting the virus to cells or tissues that have a receptor or binding molecule which interacts with the targeting ligand on the surface of the retrovirus. After infection of a cell by the virus, the virus delivers its nucleic acid into the cell and after completion of reverse transcription, the retrovirus genetic material can integrate into the host cell genome.

By inserting a heterologous polynucleotide of interest into the viral vector of the disclosure, along with another gene which encodes, for example, the ligand for a receptor on a specific target cell, the vector is now target specific. Viral vectors can be made target specific by attaching, for example, a sugar, a glycolipid, or a protein. Those of skill in the art will know of, or can readily ascertain, specific polynucleotide sequences which can be inserted into the viral genome or proteins which can be attached to a viral envelope to allow target specific delivery of the viral vector containing the nucleic acid sequence of interest.

Thus, the disclosure includes in one embodiment, a chimeric ENV protein comprising a retroviral ENV protein operably linked to a targeting polypeptide. The targeting polypeptide can be a cell specific receptor molecule, a ligand for a cell specific receptor, an antibody or antibody fragment to a cell specific antigenic epitope or any other ligand easily identified in the art which is capable of binding or interacting with a target cell. It should be noted that the antibody, antibody fragment or binding domain forming the chimeric ENV is separate and distinct from a heterologous gene downstream of a 2A or 2A-like peptide coding sequence with or without a SSP that may include a coding sequence for an antibody, antibody fragment or binding domain. Examples of targeting polypeptides or molecules include bivalent antibodies using biotin-streptavidin as linkers (Etienne-Julan et al., J. Of General Virol., 73, 3251-3255 (1992); Roux et al., Proc. Natl. Acad. Sci USA 86, 9079-9083 (1989)), recombinant virus containing in its envelope a sequence encoding a single-chain antibody variable region against a hapten (Russell et al., Nucleic Acids Research, 21, 1081-1085 (1993)), cloning of peptide hormone ligands into the retrovirus envelope (Kasahara et al., Science, 266, 1373-1376 (1994)), chimeric EPO/env constructs (Kasahara et al., 1994), single-chain antibody against the low density lipoprotein (LDL) receptor in the ecotropic MLV envelope, resulting in specific infection of HeLa cells expressing LDL receptor (Somia et al., Proc. Natl. Acad. Sci USA, 92, 7570-7574 (1995)), similarly the host range of ALV can be altered by incorporation of an integrin ligand, enabling the virus to now cross species to specifically infect rat glioblastoma cells (Valsesia-Wittmann et al., J. Virol. 68, 4609-4619 (1994)), and Dornberg and co-workers (Chu and Dornburg, J. Virol 69, 2659-2663 (1995); M. Engelstadter et al. Gene Therapy 8,1202-1206 (2001)) have reported tissue-specific targeting of spleen necrosis virus (SNV), an avian retrovirus, using envelopes containing single-chain antibodies directed against tumor markers.

The disclosure provides a method of producing a recombinant retrovirus capable of infecting a target cell comprising transfecting a suitable host cell with the following: a vector comprising a polynucleotide sequence encoding a viral gag, a viral pol and a viral env, a 2A peptide or 2A peptide-like coding sequence, an SSP coding sequence operably linked and between the 2A peptide or 2A peptide like coding sequence and a heterologous polynucleotide, wherein the 2A peptide or 2A peptide-like coding sequence is downstream of the env, packaging and psi sequences and recovering the recombinant virus.

The retrovirus and methods of the disclosure provide a replication competent retrovirus that does not require helper virus or additional nucleic acid sequence or proteins in order to propagate and produce virion. For example, the nucleic acid sequences of the retrovirus of the disclosure encode a group specific antigen and reverse transcriptase, (and integrase and protease-enzymes necessary for maturation and reverse transcription), respectively, as discussed above. The viral gag and pol can be derived from a lentivirus, such as HIV or an oncoretrovirus or gammaretrovirus such as MoMLV. In addition, the nucleic acid genome of the retrovirus of the disclosure includes a sequence encoding a viral envelope (ENV) protein. The env gene can be derived from any retroviruses. The env may be an amphotropic envelope protein which allows transduction of cells of human and other species, or may be an ecotropic envelope protein, which is able to transduce only mouse and rat cells. Further, it may be desirable to target the recombinant virus by linkage of the envelope protein with an antibody or a particular ligand for targeting to a receptor of a particular cell-type. As mentioned above, retroviral vectors can be made target specific by inserting, for example, a glycolipid, or a protein. Targeting is often accomplished by using an antibody to target the retroviral vector to an antigen on a particular cell-type (e.g., a cell type found in a certain tissue, or a cancer cell type). Those of skill in the art will know of, or can readily ascertain without undue experimentation, specific methods to achieve delivery of a retroviral vector to a specific target. In one embodiment, the env gene is derived from a non-retrovirus (e.g., CMV or VSV). Examples of retroviral-derived env genes include, but are not limited to: Moloney murine leukemia virus (MoMuLV), Harvey murine sarcoma virus (HaMuSV), murine mammary tumor virus (MuMTV), gibbon ape leukemia virus (GaLV), human immunodeficiency virus (HIV) and Rous Sarcoma Virus (RSV). Other env genes such as Vesicular stomatitis virus (VSV) (Protein G), cytomegalovirus envelope (CMV), or influenza virus hemagglutinin (HA) can also be used.

In one embodiment, the retroviral genome is derived from an onco-retrovirus, and more particularly a mammalian oncoretrovirus. In a further embodiment, the retroviral genome is derived from a gamma retrovirus, and more particularly a mammalian gamma retrovirus. By “derived” is meant that the parent polynucleotide sequence is a wild-type oncovirus which has been modified by insertion or removal of naturally occurring sequences (e.g., insertion of 2A peptide or 2A peptide like coding sequence, an SSP coding sequence and a heterologous polynucleotide encoding a polypeptide and optionally one or more of an IRES, or polIII promoter linked to another heterologous polynucleotide or an inhibitory nucleic acid of interest, respectively).

In another embodiment, the disclosure provides retroviral vectors that are targeted using regulatory sequences. Cell- or tissue-specific regulatory sequences (e.g., promoters) can be utilized to target expression of gene sequences in specific cell populations. Suitable mammalian and viral promoters for the disclosure are described elsewhere herein. Accordingly, in one embodiment, the disclosure provides a retrovirus having tissue-specific promoter elements at the 5′ end of the retroviral genome. Typically, the tissue-specific regulatory elements/sequences are in the U3 region of the LTR of the retroviral genome, including for example cell- or tissue-specific promoters and enhancers to neoplastic cells (e.g., tumor cell-specific enhancers and promoters), and inducible promoters (e.g., tetracycline).

Transcription control sequences of the disclosure can also include naturally occurring transcription control sequences naturally associated with a gene encoding a superantigen, a cytokine or a chemokine.

In some circumstances, it may be desirable to regulate expression. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV can be used. Other viral promoters that can be used include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific or selective promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. The Whey accessory protein (WAP) may be used for breast tissue expression (Andres et al., PNAS 84:1299-1303, 1987). Other promoters/regulatory domains that can be used are set forth below.

“Tissue-specific regulatory elements” are regulatory elements (e.g., promoters) that are capable of driving transcription of a gene in one tissue while remaining largely “silent” in other tissue types. It will be understood, however, that tissue-specific promoters may have a detectable amount of “background” or “base” activity in those tissues where they are expected to be silent. The degree to which a promoter is selectively activated in a target tissue can be expressed as a selectivity ratio (activity in a target tissue/activity in a control tissue). In this regard, a tissue specific promoter useful in the practice of the disclosure typically has a selectivity ratio of greater than about 5. Preferably, the selectivity ratio is greater than about 15.

In certain indications, it may be desirable to activate transcription at specific times after administration of the recombinant replication competent retrovirus of the disclosure (RRV). This may be done with promoters that are hormone or cytokine regulatable. For example, in therapeutic applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones may be used. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1990), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin. Tumor specific promoters such as osteocalcin, hypoxia-responsive element (HRE), MAGE-4, CEA, alpha-fetoprotein, GRP78/BiP and tyrosinase may also be used to regulate gene expression in tumor cells.

In addition, this list of promoters should not be construed to be exhaustive or limiting, those of skill in the art will know of other promoters that may be used in conjunction with the promoters and methods disclosed herein.

TABLE 8
TISSUE SPECIFIC PROMOTERS

| Tissue | Promoter |
|---|---|
| Pancreas | Insulin Elastin Amylase |
| | pdr-1 pdx-1 glucokinase |
| Liver | Albumin PEPCK HBV enhancer |
| | α fetoprotein apolipoprotein C α-1 |
| | antitrypsin vitellogenin, NF-AB |
| | Transthyretin |
| Skeletal muscle | Myosin H chain Muscle creatine kinase |
| | Dystrophin Calpain p94 Skeletal |
| | alpha-actin fast troponin 1 |
| Skin | Keratin K6 Keratin K1 |
| Lung | CFTR Human cytokeratin 18 (K18) |
| | Pulmonary surfactant proteins A, B |
| | and C CC-10 P1 |
| Smooth muscle | sm22 α SM-alpha-actin |
| Endothelium | Endothelin-1 E-selectin von |
| | Willebrand factor TIE (Korhonen et |
| | al., 1995) KDR/flk-1 Melanocytes |
| | Tyrosinase |
| Adipose tissue | Lipoprotein lipase (Zechner et al., |
| | 1988) Adipsin (Spiegelman et al., 1989) |
| | acetyl-CoA carboxylase (Pape and Kim, |
| | 1989) glycerophosphate dehydrogenase |
| | (Dani et al., 1989) adipocyte P2 (Hunt |
| | et al., 1986) |
| Breast | Whey Acidic Protien (WAP) (Andres et al. |
| | PNAS 84: 1299-1303 1987 |
| Blood | β-globin |

It will be further understood that certain promoters, while not restricted in activity to a single tissue type, may nevertheless show selectivity in that they may be active in one group of tissues, and less active or silent in another group. Such promoters are also termed “tissue-specific,” and are contemplated for use with the disclosure. For example, promoters that are active in a variety of central nervous system (CNS) neurons may be therapeutically useful in protecting against damage due to stroke, which may affect any of a number of different regions of the brain. Accordingly, the tissue-specific regulatory elements used in the disclosure, have applicability to regulation of the heterologous proteins as well as an applicability as a targeting polynucleotide sequence in the present retroviral vectors.

In yet another embodiment, the disclosure provides plasmids comprising a recombinant retroviral derived construct. The plasmid can be directly introduced into a target cell or a cell culture such as HT1080, NIH 3T3 or other tissue culture cells. The resulting cells release the retroviral vector into the culture medium.

The disclosure provides a polynucleotide construct comprising from 5′ to 3′: a promoter or regulatory region useful for initiating transcription; a psi packaging signal; a gag encoding nucleic acid sequence, a pol encoding nucleic acid sequence; an env encoding nucleic acid sequence; a 2A peptide or 2A peptide-like coding sequence; an SSP coding sequence; a heterologous polynucleotide encoding a marker, therapeutic or diagnostic polypeptide; an optional IRES or polIII cassette; and a LTR nucleic acid sequence. As mentioned above, the gag, pol and env nucleic acid domains can be modified to remove tryptophan codons that are converted by ApoBec3 to stop codons. In certain other embodiments, the vector may further comprise a polIII cassette or IRES cassette downstream of the heterologous polynucleotide and upstream of the 3′ LTR. As described elsewhere herein and as follows the various segment of the polynucleotide construct of the disclosure (e.g., a recombinant replication competent retroviral polynucleotide) are engineered depending in part upon the desired host cell, expression timing or amount, and the heterologous polynucleotide. A replication competent retroviral construct of the disclosure can be divided up into a number of domains that may be individually modified by those of skill in the art.

An exemplary DNA sequence for producing a recombinant retrovirus of the disclosure is provided in SEQ ID NO:2, the promoter can comprise a CMV promoter having a sequence as set forth in SEQ ID NO:2 from nucleotide 1 to about nucleotide 582 and may include modification to one or more (e.g., 2-5, 5-10, 10-20, 20-30, 30-50, 50-100 or more nucleic acid bases) so long as the modified promoter is capable of directing and initiating transcription. In one embodiment, the promoter or regulatory region comprises a CMV-R-U5 domain polynucleotide. The CMV-R-U5 domain comprises the immediately early promoter from human cytomegalovirus linked to the MLV R-U5 region. In one embodiment, the CMV-R-U5 domain polynucleotide comprises a sequence as set forth in SEQ ID NO:2 from about nucleotide 1 to about nucleotide 1202 or sequences that are at least 95% identical to a sequence as set forth in SEQ ID NO:2 wherein the polynucleotide promotes transcription of a nucleic acid molecule operably linked thereto. The gag domain of the polynucleotide may be derived from any number of retroviruses, but will typically be derived from an oncoretrovirus and more particularly from a mammalian oncoretrovirus such as MLV. In one embodiment, the gag domain comprises a sequence of SEQ ID NO:2 from about nucleotide number 1203 to about nucleotide 2819 or a sequence having at least 95%, 98%, 99% or 99.8% (rounded to the nearest 10^{th}) identity thereto. The pol domain of the polynucleotide may be derived from any number of retroviruses, but will typically be derived from an gammaretrovirus and more particularly from a mammalian gammaretrovirus such as MLV. In one embodiment the pol domain comprises a sequence of SEQ ID NO:2 from about nucleotide number 2820 to about nucleotide 6358 or a sequence having at least 95%, 98%, 99% or 99.9% (roundest to the nearest 10^{th}) identity thereto. The env domain of the polynucleotide may be derived from any number of retroviruses, but will typically be derived from a gamma-retrovirus and more particularly from a mammalian gamma-retrovirus such as MLV. In some embodiments the env coding domain comprises an amphotropic env domain. In one embodiment the env domain comprises a sequence of SEQ ID NO:2 from about nucleotide number 6359 to about nucleotide 8323 or a sequence having at least 95%, 98%, 99% or 99.8% (roundest to the nearest 10^{th}) identity thereto. The 2A peptide or 2A peptide-like/SSP cassette is inserted after the env domain (e.g., at about nucleotide 8324) and continues to the end of a heterologous polynucleotide. Examples of suitable SSP peptide are provided in Tables B and C. The heterologous domain may be followed by a polypurine rich domain or may be followed by an IRES cassette or polIII cassette. The 3′ LTR can be derived from any number of retroviruses, typically a gammaretrovirus and more typically a mammalian gammaretrovirus such as MLV. In one embodiment, the 3′ LTR comprises a U3-R-U5 domain. In yet another embodiment the LTR comprises a sequence as set forth in SEQ ID NO:2 from about nucleotide 9111 to about 11654 or a sequence that is at least 95%, 98% or 99.5% (rounded to the nearest 10^{th}) identical thereto.

TABLE B
Ranking of natural eukaryotic signal peptides by HMM cores.

| | HMM |
|---|---|
| HMM peptide sequence | Score |
| Cystatin S | 23.2 |
| Plasma protease C1 inhibitor | 21.9 |
| Erythropoietin | 21.1 |
| Lactotransferrin | 21.9 |
| Apolipoprotein C-III | 20.9 |
| MCP-1 | 20.8 |
| Alpha-2-HS-glycoprotein | 20.7 |
| Complement C3 | 20.2 |
| Vitronectin | 20.2 |
| Alpha-1-microglobulin | 20.4 |
| Lymphotoxin-alpha | 20.1 |
| Azurocidin | 19.9 |
| VIP | 19.8 |
| Metalloproteinase inhibitor 2 | 19.8 |
| Glypican-1 | 19.7 |
| Complement C1Q | 19.7 |
| Pancreatic hormone | 19.6 |
| Clusterin | 19.5 |
| Hepatocyte growth factor | 19.5 |
| Apolipoprotein E (APO-E) | 19.2 |
| Alpha-1-antichymotrypsin | 19.1 |
| Insulin | 19.4 |
| Growth hormone | 19.0 |
| Type IV collagenase | 19.0 |
| Guanylin | 18.8 |
| Proenkephalin A | 18.8 |
| Inhibin beta A chain | 18.7 |
| Properdin | 18.8 |
| Prealbumin | 18.7 |
| Angiogenin | 18.7 |
| Lutropin beta chain | 18.6 |
| Proactivator polypeptide | 18.6 |
| Fibrinogen beta chain | 18.5 |
| IGFBP 2 | 18.6 |
| Triacylglycerol lipase, gastric | 18.5 |
| Midkine | 18.4 |
| Neutrophil defensins 1, 2, and 3 | 18.4 |
| Matrix gla-protein (MGP) | 18.3 |
| Alpha-tryptase | 18.2 |
| Alpha-1-antitrypsin | 18.3 |
| Bile-salt-activated lipase | 18.2 |
| Chymotrypsinogen B | 18.2 |
| Elastin | 18.2 |
| IG lambda chain V region (4A) | 18.2 |
| Platelet factor 4 variant | 18.1 |
| Chromogranin A | 17.9 |
| WNT-1 Proto-oncogene protein | 17.9 |
| IGFBP1 | 17.8 |
| Oncostatin M (OSM) | 17.8 |
| Beta-neoendorphin-dynorphin | 17.8 |
| Von willebrand factor | 17.7 |
| Plasma serine protease inhibitor | 17.7 |
| Serum amyloid A protein | 17.6 |
| Nidogen (entactin) | 17.6 |
| Osteonectin | 17.3 |
| Histatin 3 | 17.3 |
| Phospholipase A2 | 17.3 |
| Cartilage matrix protein | 17.1 |
| GM-CSF | 17.1 |
| Matrilysin | 17.0 |
| MIP-2-beta | 17.0 |
| Neuroendocrine protein 7B2 | 16.9 |
| Interleukin-5 (IL-5) | 16.9 |
| Placental protein 11 | 16.9 |
| Gelsolin | 16.8 |
| IGF2 | 16.8 |
| M-CSF | 16.8 |
| Transcobalamin I | 16.8 |
| Lactase-phlorizin hydrolase | 16.7 |
| Elastase 2B | 16.7 |
| Pepsinogen A | 16.7 |
| MIP 1-beta | 16.6 |
| Prolactin | 16.6 |
| Trypsinogen II | 16.6 |
| Gastrin-releasing peptide II | 16.6 |
| Atrial natriuretic factor | 16.5 |
| Secreted alkaline phosphatase | 16.4 |
| Alpha-amylase pancreatic | 16.3 |
| Secretogranin I | 16.3 |
| Beta casein | 16.3 |
| Serotransferrin | 16.2 |
| Tissue factor pathway inhibitor | 16.2 |
| Follitropin beta chain | 16.2 |
| Coagulation factor XII | 16.2 |
| Growth hormone-releasing factor | 16.1 |
| Prostate seminal plasma protein | 16.0 |
| Interleukin-8 (IL-8) | 15.9 |
| Inhibin alpha chain | 15.8 |
| Angiotensinogen | 15.8 |
| Thyroglobulin | 15.7 |
| IG heavy chain | 15.6 |
| Plasminogen activator inhibitor-1 | 15.5 |
| Lysozyme C | 15.5 |
| Plasminogen activator | 15.4 |
| Antileukoproteinase 1 | 15.4 |
| Statherin | 15.4 |
| Fibulin-1, Isoform B | 15.3 |
| Fibrinogen | 15.2 |
| Uromodulin | 15.1 |
| Interleukin-4 (IL-4) | 15.1 |
| Thyroxine-binding globulin | 15.1 |
| Axonin-1 | 15.0 |
| Endometrial alpha-2 globulin | 15.0 |
| Interferon beta | 14.9 |
| Beta-2-microglobulin | 14.8 |
| Procholecystokinin (CCK) | 14.8 |
| Progastricsin | 14.7 |
| Prostatic acid phosphatase | 14.7 |
| Bone sialoprotein II | 14.6 |
| Interleukin-9 (IL-9) | 14.5 |
| Interleukin-11 (IL-11) | 14.5 |
| Colipase | 14.5 |
| Interleukin-3 (IL-3) | 14.4 |
| Alzheimer's amyloid A4 protein | 14.4 |
| PDGF, B chain | 14.2 |
| Coagulation factor V | 14.1 |
| Triacylglycerol lipase | 14.1 |
| Haptoglobin-2 | 14.1 |
| Interleukin-12 alpha chain (IL-12A) | 14.1 |
| Corticosteroid-binding globulin | 14.1 |
| Triacylglycerol lipase | 14.0 |
| Prorelaxin H2 | 14.0 |
| Follistatin 1 and 2 | 13.8 |
| Complement C1Q, A chain | 13.8 |
| Platelet glycoprotein IX | 13.8 |
| GCSF | 13.7 |
| VEGF | 13.6 |
| Heparin cofactor II | 13.6 |
| Antithrombin-III (ATIII) | 13.6 |
| Leukemia inhibitory factor (LIF) | 13.4 |
| Interstitial collagenase | 13.4 |
| Pleiotrophin (PTN) | 13.4 |
| Small inducible cytokine A1 | 13.3 |
| Melanin-concentrating hormone | 13.3 |
| Angiotensin-converting enzyme | 13.1 |
| Interleukin-2 (IL-2) | 12.6 |
| Pancreatic trypsin inhibitor | 12.6 |
| Interleukin-12 beta chain (IL-12B) | 12.6 |
| Interferon gamma | 12.6 |
| Coagulation factor VIII | 12.5 |
| Fibrinogen gamma-B chain | 12.5 |
| Interferon alpha-7 | 12.4 |
| Alpha-fetoprotein | 12.1 |
| Alpha-lactalbumin | 12.1 |
| Semenogelin II | 11.9 |
| Kappa casein | 11.9 |
| Glucagon | 11.9 |
| Thyrotropin beta chain | 11.9 |
| Transcobalamin II | 11.8 |
| Thrombospondin 1 | 11.7 |
| Parathyroid hormone (PTH) | 11.7 |
| Vasopressin copeptin | 11.6 |
| IL-1 Receptor antagonist protein | 11.6 |
| Tissue factor (TF) | 11.6 |
| Motilin | 11.5 |
| MPIF-1 | 11.5 |
| Interleukin-7 (IL-7) | 11.2 |
| Kininogen, LMW | 11.2 |
| Coagulation factor XIII B chain | 11.2 |
| Neuroendocrine convertase 2 | 10.8 |
| Complement component C7 | 10.7 |
| Stem cell factor (SCF) | 10.6 |
| Procollagen alpha 2(IV) chain | 10.4 |
| Plasma kallikrein (EC 3.4.21.34) | 10.4 |
| Interleukin-6 (IL-6) | 10.3 |
| Keratinocyte growth factor (KGF) | 9.8 |

TABLE C
Ranking of artificial
signal peptides by HMM scores

| | HMM peptide sequence | HMM |
|---|---|---|
| Name | (SEQ ID NO:) | Score |
| ASP1 | MWWRLWWLLLLLLLLWPMVA | 38 |
| | (SEQ ID NO: 289) | |
| ASP2 | MRPTWAWWLFLVLLLALWAPG | 34 |
| | (SEQ ID NO: 290) | |
| ASP3 | MKVQWLLLWVLLLLVLFCSRG | 32 |
| | (SEQ ID NO: 291) | |
| ASP4 | MRPWTWVLLLLLLICAPSYA | 30 |
| | (SEQ ID NO: 292) | |
| ASP5 | MMWLWLVLLLLCLAGNVQA | 28 |
| | (SEQ ID NO: 293) | |
| ASP6 | MPPKKCLLLLLTLLLLISTTFG | 24 |
| | (SEQ ID NO: 294) | |
| ASP7 | MAGGVAGLLLALLLPSALS | 20 |
| | (SEQ ID NO: 295) | |
| ASP8 | MKLLLIFFVLVVWMGPAHR | 16 |
| | (SEQ ID NO: 296) | |
| ASP9 | MVRGVLALLLMALQMDASSG | 12 |
| | (SEQ ID NO: 297) | |
| ASP10 | MSADCSWGAAFGALLPLAAG | 8 |
| | (SEQ ID NO: 298) | |
| ASP11 | MTKHLGVLFAGFTSADVSA | 4 |
| | (SEQ ID NO: 299) | |
| ASP12 | MIFNPMVVFLFCVSNHALR | 2 |
| | (SEQ ID NO: 300) | |
| ASP13 | MDLVSWTFMEVSTLVLPKRP | 1 |
| | (SEQ ID NO: 301) | |
| ASP14 | MLAALRRACTSACRVPIKPTHLAQG | 0 |
| | (SEQ ID NO: 302) | |

The retroviral vectors can be used to treat a wide range of disease and disorders including a number of cell proliferative diseases and disorders (see, e.g., U.S. Pat. Nos. 4,405,712 and 4,650,764; Friedmann, 1989, Science, 244:1275-1281; Mulligan, 1993, Science, 260:926-932, R. Crystal, 1995, Science 270:404-410, each of which are incorporated herein by reference in their entirety, see also: The Development of Human Gene Therapy, Theodore Friedmann, Ed., Cold Spring Harbor Laboratory Press, Cold Spring Harbor, N.Y., 1999. ISBN 0-87969-528-5; Concepts in Genetic Medicine, ed. Boro Dropulic and Barrie Carter, Wiley, 2008, Hoboken, N.J.; Gene & Cell Therapy-Therapeutic Mechanism and Strategies, 3rd edition ed. Nancy Smyth Templeton, CRC Press, Boca Raton Fla. 2008; Xavier et al., Annu. Rev. Med. 70:273-88, 2019, each of which is incorporated herein by reference in its entirety).

The disclosure also provides gene therapy for the treatment of cell proliferative disorders. Such therapy would achieve its therapeutic effect by introduction of an appropriate therapeutic polynucleotide (e.g., encoding antigen binding proteins/polypeptides, cytokines, ligands, antisense, ribozymes, prodrug activating enzymes, siRNA), into cells of subject having the proliferative disorder or into allogeneic mesenchymal stem cells (MSCs), neural stem cells (NSCs) or other cell types known to be capable of targeting sites of inflammation or tumors. Delivery of polynucleotide constructs can be achieved using the recombinant retroviral vector of the disclosure, particularly if it is based on MLV or other gammaretrovirus, which are capable of infecting dividing cells.

In addition, the therapeutic methods (e.g., the gene therapy or gene delivery methods) as described herein can be performed in vivo or ex vivo. It may be preferable to remove the majority of a tumor prior to gene therapy, for example surgically or by radiation. In some aspects, the retroviral therapy may be preceded or followed by surgery, chemotherapy or radiation therapy.

Thus, the disclosure provides a recombinant retrovirus capable of infecting a non-dividing cell, a dividing cell or a neoplastic cell, therein the recombinant retrovirus comprises a viral GAG; a viral POL; a viral ENV; a heterologous nucleic acid operably linked to a 2A peptide or peptide-like coding sequence; and cis-acting nucleic acid sequences necessary for packaging, reverse transcription and integration. The recombinant retrovirus can be a lentivirus, such as HIV, or can be a gammaretrovirus.

The disclosure also provides a method of nucleic acid transfer to a target cell to provide expression of a particular nucleic acid (e.g., a heterologous sequence). Therefore, in another embodiment, the disclosure provides a method for introduction and expression of a heterologous nucleic acid in a target cell comprising infecting the target cell with the recombinant virus of the disclosure and expressing the heterologous nucleic acid in the target cell, wherein the heterologous nucleic acid is engineered into the recombination viral vector downstream of the env domain and operably linked to a 2A or 2A like-peptide-SSP construct. As mentioned above, the target cell can be any cell type including dividing, non-dividing, neoplastic, immortalized, modified and other cell types recognized by those of skill in the art, so long as they are capable of infection by a retrovirus.

It may be desirable to transfer a nucleic acid encoding a biological response modifier (e.g., a cytokine) into a cell or subject. Included in this category are immunopotentiating agents including nucleic acids encoding a number of the cytokines classified as “interleukins”. These include, for example, interleukins 1 through 38, as well as other response modifiers and factors described elsewhere herein. Also included in this category, although not necessarily working according to the same mechanisms, are interferons, and in particular gamma interferon, tumor necrosis factor (TNF) and granulocyte-macrophage-colony stimulating factor (GM-CSF). Other polypeptides include, for example, angiogenic factors and anti-angiogenic factors. It may be desirable to deliver such nucleic acids to bone marrow cells or macrophages to treat enzymatic deficiencies or immune defects. Nucleic acids encoding growth factors, toxic peptides, ligands, receptors, or other physiologically important proteins can also be introduced into specific target cells. Any of the foregoing biological response modifiers are engineered into the RRV of the disclosure downsream and operably liked to the 2A or 2A like-peptide-SSP construct.

The disclosure can be used for delivery of heterologous polynucleotides that promotes drug specific targeting and effects. For example, HER2, a member of the EGF receptor family, is the target for binding of the drug trastuzumab (Herceptin™, Genentech). Trastuzumab is a mediator of antibody-dependent cellular cytotoxicity (ADCC). Activity is preferentially targeted to HER2-expressing cells with 2+ and 3+ levels of overexpression by immunohistochemistry rather than 1+ and non-expressing cells (Herceptin prescribing information, Crommelin 2002). Enhancement of expression of HER2 by introduction of vector expressing HER2 or truncated HER2 (expressing only the extracellular and transmembrane domains) in HER2 low tumors may facilitate optimal triggering of ADCC and overcome the rapidly developing resistance to Herceptin that is observed in clinical use. In these instances the heterologous gene would encode HER2.

In another example, CD20 is the target for binding of the drug rituximab (Rituxan™, Genentech). Rituximab is a mediator of complement-dependent cytotoxicity (CDC) and ADCC. Cells with higher mean fluorescence intensity by flow cytometry show enhanced sensitivity to rituximab (van Meerten et al., Clin Cancer Res 2006; 12(13):4027-4035, 2006). Enhancement of expression of CD20 by introduction of vector expressing CD20 in CD20 low B cells may facilitate optimal triggering of ADCC. In this instance the heterologous gene encodes CD20.

The disclosure provides methods for treating cell proliferative disorders such as cancer and neoplasms comprising administering an RRV vector of the disclosure followed by treatment with a chemotherapeutic agent or anti-cancer agent. In one embodiment, the RRV vector is administered to a subject for a period of time prior to administration of the chemotherapeutic or anti-cancer agent that allows the RRV to infect and replicate. The subject is then treated with a chemotherapeutic agent or anti-cancer agent for a period of time and dosage to reduce proliferation or kill the cancer cells. In one embodiment, if the treatment with the chemotherapeutic or anti-cancer agent reduces, but does not kill the cancer/tumor (e.g., partial remission or temporary remission), the subject may then be treated with a non-toxic therapeutic agent (e.g., 5-FC) that is converted to a toxic therapeutic agent in cells expression a cytotoxic gene (e.g., cytosine deaminase) from the RRV.

Using such methods the RRV vectors of the disclosure are spread during a replication process of the tumor cells, such cells can then be killed by treatment with an anti-cancer or chemotherapeutic agent and further killing can occur using the RRV treatment process described herein.

In yet another embodiment of the disclosure, the heterologous gene can comprise a coding sequence for a target antigen (e.g., a cancer antigen). In this embodiment, cells comprising a cell proliferative disorder are infected with an RRV comprising a heterologous polynucleotide encoding the target antigen to provide expression of the target antigen (e.g., overexpression of a cancer antigen). An anticancer agent comprising a targeting cognate moiety that specifically interacts with the target antigen is then administered to the subject. The targeting cognate moiety can be operably linked to a cytotoxic agent or can itself be an anticancer agent. Thus, a cancer cell infected by the RRV comprising the targeting antigen coding sequences increases the expression of target on the cancer cell resulting in increased efficiency/efficacy of cytotoxic targeting.

In yet another embodiment, an RRV of the disclosure can comprise a coding sequence comprising a binding domain (e.g., an antibody, antibody fragment, antibody domain, non-antibody binding domain or receptor ligand) that specifically interacts with a cognate antigen or ligand. The RRV comprising the coding sequence for the binding domain can then be used to infect cells in a subject comprising a cell proliferative disorder such as a cancer cell or neoplastic cell. The infected cell will then express the binding domain or antibody. An antigen or cognate operably linked to a cytotoxic agent or which is cytotoxic itself can then be administered to a subject. The cytotoxic cognate will then selectively kill infected cells expressing the binding domain. Alternatively the binding domain itself can be an anti-cancer agent that, for example, interacts with the immune system, such as anti-PD-L1 or anti-CTLA-4.

The disclosure provides a method of treating a subject having a cell proliferative disorder. The subject can be any mammal, including a human. The subject is contacted with a recombinant replication competent retroviral vector of the disclosure. The contacting can be in vivo or ex vivo. Methods of administering the retroviral vector of the disclosure are known in the art and include, for example, systemic administration, topical administration, intraperitoneal administration, intra-muscular administration, intracranial, cerebrospinal, as well as administration directly at the site of a tumor or cell-proliferative disorder. Other routes of administration known in the art can also be employed.

Thus, the disclosure includes various pharmaceutical compositions useful for treating a cell proliferative disorder. The pharmaceutical compositions according to the disclosure are prepared by bringing a retroviral vector containing a heterologous polynucleotide sequence useful in treating or modulating a cell proliferative disorder according to the disclosure into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

In other embodiments, host cells transfected with a replication competent retroviral vector of the disclosure are provided. Host cells include eukaryotic cells such as yeast cells, insect cells, or animal cells. Host cells also include prokaryotic cells such as bacterial cells.

Also provided are engineered host cells that are transduced (transformed or transfected) with a vector provided herein (e.g., a replication competent retroviral vector). The engineered host cells can be cultured in conventional nutrient media modified as appropriate for activating promoters, selecting transformants, or amplifying a coding polynucleotide. Culture conditions, such as temperature, pH and the like, are those previously used with the host cell selected for expression, and will be apparent to those skilled in the art and in the references cited herein, including, e.g., Sambrook, Ausubel and Berger, as well as e.g., Freshney (1994) Culture of Animal Cells: A Manual of Basic Technique, 3rd ed. (Wiley-Liss, New York) and the references cited therein.

Examples of appropriate expression hosts include: bacterial cells, such as E. coli, B. subtilis, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 br Bowes melanoma; or plant cells or explants, etc. Typically human cells or cell lines will be used; however, it may be desirable to clone vectors and polynucleotides of the disclosure into non-human host cells for purposes of sequencing, amplification and cloning.

The following Examples are intended to illustrate, but not to limit the disclosure. While such Examples are typical of those that might be used, other procedures known to those skilled in the art may alternatively be utilized.

### EXAMPLES

### Example 1: Design of RRV-2A-GFPm, RRV-GSG-2A, RRV-2A-yCD2 and RRV-GSG-2A-yCD2

RRV-yCD2 and RRV-GFP are Moloney MLV-based RRVs with an amphotropic envelope gene and an encephalomyocarditis virus internal ribosome entry site (IRES)—transgene cassette downstream of the env gene (Perez et al, 2012). RRV-2A-GFP (aka pAC3-2A-GFP) and RRV-2A-yCD2 (pAC3-2A-yCD2) vectors are based on RRV-GFP and RRV-yCD2 but the IRES region has been replaced with a variety of different 2A peptides in-frame with the amphotropic envelope protein and the transgene (GFP or yCD2). The cloning scheme for RRV-2A-GFP and RRV-yCD2 vectors has been described previously (Hofacre et al Hum. Gene Ther. 29:437-451 2018. Briefly, a pAC3-T2A-GFP construct was first generated using Gibson Assembly Cloning Kit (NEB) containing 2 DNA fragments and pAC3-emd backbone digested with BstB I and Not I site. First, a pair of sense and antisense oligonucleotides containing sequence of the 3′ end of the amphotropic env, 2A peptide from Thosea asigna virus (T2A), and 5′ of GFP in 5′-to-3′ order was synthesized (IDT) and hybridized to generate DNA fragment 2A-G. The second DNA fragment in the Gibson Assembly is the FP fragment. FP fragment was generated by PCR using the following primers: GFP-F-Gib (5′-GAAGTTCGAGGGCGACAC-3′ (SEQ ID NO:303)) and GFP-R-Gib (5′-TAAAATCTTTTATTTTATCTGCGGCCGCAC-3′ (SEQ ID NO:304)).

In the 2A-G fragment, the 5′contains sequence that overlaps with the BstBI site in the amphotropic env of the pAC3 backbone; the 3′ contains sequence that overlaps with the 5′ of the FP DNA fragment. In addition, AscI restriction enzyme site was placed at the 3′-end of T2A, immediately upstream of the start codon for the second transgene, GFP. The inclusion of AscI site is for subsequent replacement of the T2A peptide with other 2A peptides. The inclusion of AscI restriction site with an additional nucleotide T followed by the AscI site resulted in an additional 3 amino acids (glycine-alanine-proline) C-terminus to the last proline residue in the T2A peptide. During the co-translation process, the separation of the GFP protein from envelope protein mediated by the T2A peptide resulted in an additional 4 amino acids P, G, A, and P at the N-terminus of the GFP. In the FP fragment, the 5′-end of the FP fragment contains sequence which overlaps to the 3′-end of the 2A-G fragment by 24 nucleotides and the 3′-end of the FP fragment overlaps the 5′-end of the pAC3-GFP backbone spanning the Not I site by 26 nucleotides. The resulting plasmid DNA from Gibson Assembly Cloning was designated pAC3-T2A-GFP.

Additional RRV-2A-GFP vectors harboring three other commonly used 2A peptides derived from Porcine teschovirus-1 (P2A), Foot-and-mouth disease virus (F2A), and Equine rhinitis A virus (E2A), in two different configurations, were subsequently synthesized (IDT). Each DNA fragment contains sequence of 3′ of amphotropic env gene and the designated 2A peptide in place of the T2A of the pAC3-T2A-GFP backbone at the BstBI and AscI site. The resulting plasmid DNA are designated pAC3-P2A-GFP, pAC3-F2A-GFP, pAC3-E2A-GFP, pAC3-GSG-T2A-GFP, pAC3-GSG-P2A-GFP, pAC3-GSG-F2A-GFP, and pAC3-GSG-E2A-GFP.

It was later determined that RRV-2A-GFP plasmid DNAs described (pAC3-E2A-GFP, pAC3-F2A-GFP, pAC3-P2A-GFP, pAC3-T2A-GFP, pAC3-GSG-E2A-GFP, pAC3-GSG-F2A-GFP, pAC3-GSG-P2A-GFP, and pAC3-GSG-T2A-GFP) all contained a stop codon mutation at the 3′-end of GFP. The mutation was introduced in the GFP-R-Gib primer (5′-TAAAATCTTTTATTTTATCTGCGGCCGCAC-3′ (SEQ ID NO:4)) when generating the FP PCR fragment. The stop codon mutation in the GFP derived from PCR resulted in read through of the GFP ORF for additional 11 amino acids (C-A-A-A-D-K—I-K-D-F-I (SEQ ID NO:5)) before reaching to a stop codon. The plasmids DNA were re-designated as pAC3-E2A-GFPm, pAC3-F2A-GFPm, pAC3-P2A-GFPm, pAC3-T2A-GFPm, pAC3-GSG-E2A-GFPm, pAC3-GSG-F2A-GFPm, pAC3-GSG-P2A-GFPm, and pAC3-GSG-T2A-GFPm. Hereafter, the two nomenclatures pAC3-E2A-GFP/pAC3-E2A-GFPm, pAC3-F2A-GFP/pAC3-F2A-GFPm, pAC3-P2A-GFP/pAC3-P2A-GFPm, pAC3-T2A-GFP/pAC3-T2A-GFPm, pAC3-GSG-E2A-GFP/pAC3-GSG-E2A-GFPm, pAC3-GSG-F2A-GFP/pAC3-GSG-F2A-GFPm, pAC3-GSG-P2A-GFP/pAC3-GSG-P2A-GFPm, and pAC3-GSG-T2A-GFP/pAC3-GSG-T2A-GFPm are used interchangeably.

An equivalent set of 4 RRV-2A-yCD2 vectors were generated by replacing the GFPm open reading frame with yCD2 ORF in the respective 2A peptide version of pAC3-P2A-GFPm, pAC3-GSG-P2A-GFPm, pAC3-T2A-GFPm and pAC3-GSG-T2A-GFPm plasmid DNA. The AscI-yCD2-NotI PCR fragment was generated from the pAC3-yCD2 plasmid DNA using the primers: AscI-yCD2-F (5′-GATCGGCGCGCCTATGGTGACCGGCGGCATGGC-3′ (SEQ ID NO:6) and 3-37 (5′-CCCCTTTTTCTGGAGACTAAATAA-3′ (SEQ ID NO:7). The PCR product and each of the four pAC3-2A-GFPm plasmid DNAs were restriction enzyme digested with AscI and NotI, and the AscI-yCD2-NotI digested PCR product was subcloned in place of GFPm to generate pAC3-P2A-yCD2, pAC3-GSG-P2A-yCD2. pAC3-T2A-yCD2, and pAC3-GSG-T2A-yCD2 (Table DL

TABLE D
Sequence, source of the 2A peptide,
and RRV plasmid-2A peptide-transgene name.

| Nucleotide sequence | Source of 2A | |
|---|---|---|
| (GSG-linker sequence underlined) | (infected species) | |
| | | RRV-2A-GFP |
| | | plasmid |
| GAGGGCAGAGGAAGTCTTCTAACATGCGGTGACGTG | Thosea asigna virus | pAC3-T2A-GFP |
| GAGGAGAATCCCGGCCCT (SEQ ID NO: 8) | (insects) | |
| GGAAGCGGAGAGGGCAGAGGAAGTCTTCTAACATGC | Thosea asigna virus | pAC3-GSG-T2A-GFP |
| GGTGACGTGGAGGAGAATCCCGGCCCT (SEQ ID | (insects) | |
| NO: 9) | | |
| GCTACTAACTTCAGCCTGCTGAAGCAGGCTGGAGAC | Porcine teschovirus-1 | pAC3-P2A-GFP |
| GTGGAGGAGAACCCTGGACCT (SEQ ID NO: 10) | (mammals) | |
| GGAAGCGGAGCTACTAACTTCAGCCTGCTGAAGCAG | Porcine teschovirus-1 | pAC3-GSG-P2A-GFP |
| GCTGGAGACGTGGAGGAGAACCCTGGACCT (SEQ ID | (mammals) | |
| NO: 11) | | |
| GTGAAACAGACTTTGAATTTTGACCTTCTCAAGTTG | Foot-and-mouth | pAC3-F2A-GFP |
| GCGGGAGACGTGGAGTCCAACCCTGGACCT (SEQ ID | disease virus(mammals) | |
| NO: 12) | | |
| GGAAGCGGAGTGAAACAGACTTTGAATTTTGACCTT | Foot-and-mouth | pAC3-GSG-F2A-GFP |
| CTCAAGTTGGCGGGAGACGTGGAGTCCAACCCTGGACCT | disease virus | |
| (SEQ ID NO: 13) | (mammals) | |
| CAGTGTACTAATTATGCTCTCTTGAAATTGGCTGGA | Equine rhinitis A virus | pAC3-E2A-GFP |
| GATGTTGAGAGCAACCCTGGACCT (SEQ ID NO: 14) | (mammals) | |
| GGAAGCGGACAGTGTACTAATTATGCTCTCTTGAAA | Equine rhinitis A virus | pAC3-GSG-E2A-GFP |
| TTGGCTGGAGATGTTGAGAGCAACCCTGGACCT (SEQ | (mammals) | |
| ID NO: 15) | | |
| | | RRV-2A-yCD2 |
| | | plasmid |
| GAGGGCAGAGGAAGTCTTCTAACATGCGGTGACGTG | Thosea asigna virus | pAC3-T2A-yCD2 |
| GAGGAGAATCCCGGCCCT (SEQ ID NO: 16) | (insects) | |
| GGAAGCGGAGAGGGCAGAGGAAGTCTTCTAACATGC | Thosea asigna virus | pAC3-GSG-T2A-yCD2 |
| GGTGACGTGGAGGAGAATCCCGGCCCT (SEQ ID | (insects) | |
| NO: 17) | | |
| GCTACTAACTTCAGCCTGCTGAAGCAGGCTGGAGAC | Porcine teschovirus-1 | pAC3-P2A-yCD2 |
| GTGGAGGAGAACCCTGGACCT (SEQ ID NO: 18) | (mammals) | |
| GGAAGCGGAGCTACTAACTTCAGCCTGCTGAAGCAG | Porcine teschovirus-1 | pAC3-GSG-P2A-yCD2 |
| GCTGGAGACGTGGAGGAGAACCCTGGACCT (SEQ ID | (mammals) | |
| NO: 19) | | |

### Example 2: RRV-2A-GFPm and RRV-GSG-2A-GFPM Vectors Produced from 293T Cells are Infectious and Express GFP Protein

HEK293T cells were seeded at 2e6 cells per 10 cm plates, 18 to 20 hours pre transfection. The next day, pAC3-2A-GFPm and pAC3-GSG-2A-GFPm plasmids were used for transient transfection of 20 μg of plasmid DNA at 20 h post-cell seeding using the calcium phosphate method. Eighteen hours post transfection, cells were washed with DMEM complete medium three times and incubated with fresh complete culture medium. Viral supernatant was collected approximately 42 h post-transfection and filtered through a 0.45 μm syringe filter. The viral titers of RRV-2A-GFPm, RRV-GSG-2A-GFPm and RRV-IRES-GFP from transient transfection of HEK293T cells were determined as described previously (Perez et al., 2012). Briefly, vector preparations titers were determined on PC3 cells by single-cycle infection of the vector. The single-cycle infection was guaranteed by azidothymidine treatment 24 h post-infection, followed by quantitative PCR (qPCR) of target cell genomic DNA specific for viral vector DNA (MLV LTR primer set; 5-MLV-U3-R (5′-AGCCCACAACCCCTCACTC-3′ (SEQ ID NO:20)), 3-MLV-Psi (5′-TCTCCCGATCCCGGACGA-3′ (SEQ ID NO:21)), and probe (5′-FAM-CCCCAAATGAAAGACCCCCGCTGACG-BHQ1-3′ (SEQ ID NO:22)) 48 h post-infection, to quantify the number of viral DNA copies per cell genome. Viral titers, reported in transduction units (TU) per milliliter (TU/mL), were determined by calculation of threshold cycle (CT) values derived from a standard curve ranging from 2×10⁷ copies to 2×10¹copies of plasmid DNA and from a known amount of genomic DNA input, the number of cells, and a dilution of the viral stock per reaction mixture. Table E shows that titers of RRV-2A-GFPm and RRV-GSG-2A-GFPm produced from HEK293T cells were comparable to that of RRV-IRES-GFP.

TABLE E
Titers of RRV-2A-GFPm and RRV-GSG-2A-
GFPm vectors produced from 293T cells

| | TU/mL | Stdv |
|---|---|---|
| pAC3-E2A-GFP | 1.15E+06 | 2.55E+05 |
| pAC3-F2A-GFP | 1.63E+06 | 2.58E+05 |
| pAC3-P2A-GFP | 1.81E+06 | 3.11E+05 |
| pAC3-T2A-GFP | 3.31E+06 | 1.32E+05 |
| pAC3-GSG-E2A-GFP | 1.65E+06 | 2.76E+05 |
| pAC3-GSG-F2A-GFP | 1.32E+06 | 7.57E+04 |
| pAC3-GSG-P2A-GFP | 1.31E+06 | 1.22E+05 |
| pAC3-GSG-T2A-GFP | 2.66E+06 | 2.14E+05 |
| pAC3emd | 1.65E+06 | 2.12E+05 |

The RRV-2A-GFPm viruses produced from HEK293T cells were then used to infect U87-MG at a multiplicity of infection (MOI) of 0.01. U87-MG cells were seeded at 1×10⁵ cells in 6-well plates for initial infection. The cells were passaged to a new well of a 6-well plate at a dilution of 1 to 4 at each passage and the remainder of the cells from each sample was harvested to assess viral spread by measuring percent of GFPm expressing cells and GFPm mean fluorescent intensity using BD FACS Canto II (BD Biosciences). The percentages of GFP-positive cells at each passage were plotted. The length of the assay was carried out until all RRV-2A-GFP viruses reached to maximum infectivity (˜95% or greater GFP-positive cells). The rate of viral spread among RRV-2A-GFPm and RRV-GSG-2A-GFPm were similar to RRV-IRES-GFP in infected U87-MG cells, with the exception of RRV-P2A-GFPm, RRV-T2A-GFPm and RRV-GSG-F2A-GFPm exhibiting a lag. Nevertheless, they reached maximally infectivity within 18 days. The GFPm expression levels also varied among RRV-2A-GFPm and RRV-GSG-2A-GFPm vectors but were all at approximately 20 to 50% of that expressed from RRV-IRES-GFP infected U87-MG cells.

### Example 3: RRV-2A-GFPm and RRV-GSG-2A-GFPm Vectors are Stable in U87-MG Cells

To ensure that the reduced GFP expression in RRV-2A-GFPm and RRV-GSG-2A-GFPm infected U87-MG cells is not due to deletion of GFP gene in viral genome, the integrity of 2A-GFPm region was assessed by end-point PCR using primer set which span the 3′env and 3′UTR region of proviral DNA. At maximal infectivity of the U87-MG cells, cells were subsequently cultured to reach confluency in a T75 flask, at which time the media was replaced with fresh media, followed by the collection of virus containing supernatant and 0.45 μM filtration at 18-24 h post media change. The collected cell supernatant was aliquoted and stored at −80° C. until being used for immunoblotting and re-infection experiments. At the same time, the cells were split into two fractions; 1/10^{th} for isolation of genomic DNA and 9/10^{th} for isolation of total cell lysates. The genomic DNA was extracted from the cell pellet by resuspending in 400 μL 1×PBS and isolated using the Promega Maxwell 16 Cell DNA Purification Kit (Promega). One-hundred nanogram of genomic DNA was then use as the template for PCR with a primer set: IRES-F (5′-CTGATCTTACTCTTTGGACCTTG-3′(SEQ ID NO:23)) and IRES-R (5′-CCCCTTTTTCTGGAGACTAAATAA-3′ (SEQ ID NO:24)). The resultant PCR products were analyzed on 1% agarose gel. The data show that the 2A-GFPm and GSG-2A-GFPm region in proviral DNA of RRV-2A-GFPm and RRV-GSG-2A-GFPm vectors are stable in U87-MG cells during the time course of viral replication.

### Example 4: RRV-2A-GFPm and RRV-GSG-2A-GFPm Produced from Maximally Infected U87-MG Cells Remain Infectious in the Subsequently Infection Cycle

As long-term infectivity is one of the many important criteria to sustain therapeutic effect delivered by RRV, infectivity of RRV-2A-GFPm and RRV-GSG-2A-GFPm produced from maximally infected U87-MG cells was evaluated by performing an additional cycle of infection in naïve U87-MG cells. Viral supernatants collected from maximally infected U87-MG cells were first titered as described then re-infected back onto naïve U87-MG cells at an MOI of 0.01. Titers produced from maximally infected U87-MG cells were similar to those obtained from transiently transfected HEK293T cells are comparable among RRV-2A-GFPm, RRV-GSG-2A-GFPm vectors as well as RRV-IRES-GFP vector.

The viral spread of RRV-2A-GFPm and RRV-GSG-2A-GFPm was monitored at each cell passage as described. In contrast to the viral spread rate observed in the first infection cycle using the viral supernatant produced from transiently transfected HEK293T cells, all vectors spread at the rate comparable to RRV-IRES-GFP. However, the GFP expression levels from RRV-2A-GFPm and RRV-GSG-2A-GFPm infected U87-MG cells in this infection cycle remained at 20 to 50% of that expressed by RRV-IRES-GFP cells, as previously observed.

### Example 5: The Viral Envelope and GFPm Proteins of RRV-2A-GFPm and RRV-GSG-2A-GFPm Vectors are Processed at Different Efficiency in Infected U87-MG Cells

To assess the GFPm expression, the separation efficiency of GFPm from the viral envelope protein, and the proper processing of the viral envelope protein, cell lysates were generated from infected U87-MG cells. U87-MG cells at maximal infectivity, confluent cell monolayer was washed once in 1×PBS, disassociated by TrpZean (Sigma), resuspended in complete DMEM, washed again in 1×PBS, followed by cell lysis in 200 μL of RIPA lysis buffer (Thermo Scientific) on ice for 30 minutes. The lysates were clarified of cellular debris by centrifugation at 14,000 rpm for 15 m at 4° C. and the supernatants collected and transferred to a new tube. The cell lysates were then assayed for their protein concentration using BCA precipitation assay (Thermo Scientific) and 20 μg protein was subjected to SDS-PAGE. The proteins were resolved on 4-12% XT-Tris SDS-PAGE gels (BioRad) for 45 minutes at 200 volts. Subsequently the proteins were transferred onto PVDF membranes (Life Technologies) using an iBlot dry blotting system at 20 volts for 7 minutes. The membranes were assayed for the expression of the gp70 subunit of the envelope protein and the GFPm, using anti-gp70 (rat anti-gp70, clone 83A25; 1:500 dilution) and anti-GFP (rabbit anti-GFP; 1:1000 dilution). Protein expression was detected using the corresponding secondary antibody conjugated to horseradish peroxidase. The result show that GFPm protein from RRV-F2A-GFPm, RRV-P2A-GFPm, and RRV-T2A-GFPm, RRV-GSG-F2A-GFPm and RRV-GSG-F2A-GFPm were separated inefficiently from the viral envelope protein, as indicated by the high molecular weight of the env-2A-GFPm fusion protein at ˜120 KDa, using the anti-GFP antibody. In contrast, the separation of GFPm from the viral envelope protein was relative efficient for RRV-E2A-GFPm, RRV-GSG-P2A-GFPm and RRV-GSG-T2A-GFPm vectors compared to that from RRV-IRES-GFP. In parallel, the processing of the viral envelope protein in infected U87-MG was examined using the anti-gp70 antibody. The result show the viral enveloped in either precursor (Pr85) or processed form (gp70) were detected in all RRV-2A-GFPm and RRV-GSG-2A-GFPm vectors, suggesting separation of the viral envelope protein from the GFPm as seen in the anti-GFP immunoblot. In addition, the efficiency of separation observed in the anti-gp70 blot is somewhat consistent with that observed in the anti-GFP immunblot. Although the protein expression of the fusion polyprotein, Env-GFPm, varied among the RRV-2A-GFPm and RRV-GSG-2A-GFPm vectors, RRV-GSG-P2A-GFPm and RRV-T2A-GFPm appear to have most efficient separation as indicated by the lack of detection of the viral envelope-GFPm fusion polyprotein in both anti-GFP and anti-gp70 immunoblots.

### Example 6: The Level of Incorporation of Properly Processed Viral Envelope Protein Correlates with the Efficiency of Separation Between the Viral Envelope and GFPm Proteins

Viral supernatants from RRV-2A-GFPm and RRV-GSG-2A-GFPm maximally infected U87-MG cells were pelleted through a 20% sucrose gradient at 14000 rpm for 30 m at 4° C., and subsequently resuspended in 20 μL of 1× Laemmli Buffer containing 5% 2-mercaptoethanol and subjected to SDS PAGE on 4-20% Tris Glycine gels (BioRad). The electrophoresis and protein transfer were performed as described. Properly processed viron-associated viral envelope protein expression was examined using anti-gp70 (rat raised anti-gp70, clone 83A25; 1:500 dilution) and the anti-p15E (mouse raised anti-TM, clone 372; 1:250 dilution). Protein expression was detected using the corresponding secondary antibody conjugated to horseradish peroxidase. The data indicate that properly processed envelope protein, gp70 and p12E/p15E of RRV-2A-GFPm and RRV-GSG-2A-GFPm, except RRV-P2A-GFPm and RRV-T2A-GFPm vectors, were detected at levels comparable to that of RRV-IRES-GFP in virions. As expected, RRV-GSG-P2A-GFPm and RRV-T2A-GFPm which showed lowest level of virion-associated envelope protein expressed highest level of fusion polyprotein in cell lysates. Consistent with published data, the data support the notation that unprocessed envelope protein precursor protein Pr85 or in this case the viral envelope-GFPm fusion polyprotein does not get incorporated into virion. Furthermore, the cleavage of the R peptide bearing the 2A peptide leading to “fusogenic” p12E also appears to be sufficient during virion maturation to produce infectious viral particles as indicated by the titer produced from maximally infected U87-MG cells. The nature of p15E/p12E ratio and its role in membrane fusion during infection is unclear. All together, the data suggest that the level of viral envelope protein incorporation does not correlate with titer values measured in target cells. The unexpected lack of difference in titer values among vectors, particularly the RRV-GSG-P2A-GFPm and RRV-T2A-GFPm vectors suggests that a range of envelope expression levels can be tolerated on the RRV particles1 without affecting titer on these cells.

### Example 7: RRV-P2A-yCD2 and RRV-T2A-yCD2, RRV-GSG-P2A-yCD2 and RRV-GSG-T2A-yCD2 Vectors Produced from 293T Cells are Infectious and Express yCD2 Protein

HEK293T cells were seeded at 2e6 cells per 10 cm plates, 18 to 20 hours pre transfection. The next day, pAC3-P2A-yCD2, pAC3-T2A-yCD2, pAC3-GSG-P2A-yCD2, and pAC3-GSG-T2A-yCD2 plasmids were used for transient transfection of 20 μg of plasmid DNA at 20 h post-cell seeding using the calcium phosphate method. Eighteen hours post transfection, cells were washed with DMEM complete medium three times and incubated with fresh complete culture medium. Viral supernatant was collected approximately 42 h post-transfection and filtered through a 0.45 μm syringe filter. The viral titers of RRV-P2A-yCD2, RRV-T2A-yCD2, RRV-GSG-P2A-yCD2, and RRV-GSG-T2A-yCD2 from transient transfection of HEK293T cells were determined as described previously (Perez et al., 2012). Briefly, vector preparations titers were determined on PC3 cells by single-cycle infection of the vector. The single-cycle infection was guaranteed by azidothymidine treatment 24 h post-infection, followed by quantitative PCR (qPCR) of target cell genomic DNA specific for viral vector DNA (MLV LTR primer set; 5-MLV-U3-R (5′-AGCCCACAACCCCTCACTC-3′(SEQ ID NO:20)), 3-MLV-Psi (5′-TCTCCCGATCCCGGACGA-3′ (SEQ ID NO:21)) and probe (5′-FAM-CCCCAAATGAAAGACCCCCGCTGACG-BHQ1-3′ (SEQ ID NO:22)) 48 h post-infection, to quantify the number of viral DNA copies per cell genome. Viral titers, reported in transduction units (TU) per milliliter (TU/mL), were determined by calculation of threshold cycle (CT) values derived from a standard curve ranging from 2×10⁷ copies to 2×10¹copies of plasmid DNA and from a known amount of genomic DNA input, the number of cells, and a dilution of the viral stock per reaction mixture. Table F shows that titers of RRV-P2A-yCD2, RRV-T2A-yCD2, RRV-GSG-P2A-yCD2, and RRV-GSG-T2A-yCD2 produced from HEK293T cells were comparable to that of RRV-IRES-yCD2.

TABLE F
Titers of RRV-P2A-yCD2, RRV-T2A-yCD2, RRV-GSG-P2A-yCD2
and RRV-GSG-T2A-yCD2 vectors produced from 293T cells

| | TU/mL | Stdv |
|---|---|---|
| pAC3P2AyCD2 | 3.06E+06 | 4.59E+05 |
| pAC3GSGP2AyCD2 | 1.15E+06 | 2.45E+05 |
| pAC3T2AyCD2 | 2.32E+06 | 3.78E+05 |
| pAC3GSGT2AyCD2 | 1.88E+06 | 4.64E+05 |
| pAC3-yCD2 | 1.76E+06 | 1.84E+05 |

In addition, viral supernatants collected from maximally infected U87-MG cells were titered as described to ensure they remain infectious. The primer set used for titer have similar priming efficiency as the primer set containing the, 5-MLV-U3-R, 3-MLV-Psi primers and probe. The primer set used for tittering the RRV-P2A-yCD2, RRV-T2A-yCD2, RRV-GSG-P2A-yCD2 and RRV-GSG-T2A-yCD2 vectors from infected U87-MG cells are: Env2 For: 5′-ACCCTCAACCTCCCCTACAAGT-3′ (SEQ ID NO:25), Env2 Rev: 5′-GTTAAGCGCCTGATAGGCTC-3′ (SEQ ID NO:26) and probe 5′-FAM-CCCCAAATGAAAGACCCCCGCTGACG-BHQ1-3′ (SEQ ID NO:27). Titers produced from maximally infected U87-MG cells were similar to those obtained from transiently transfected HEK293T cells and comparable among RRV-IRES-yCD2 vector.

### Example 8: The Viral Envelope and yCD2 Proteins of RRV-P2A-yCD2 and RRV-T2A-yCD2, RRV-GSG-P2A-yCD2 and RRV-GSG-T2A-yCD2 Vectors in Infected U87-MG Cells are Processed at Different Efficiency

To assess the yCD2 expression, the separation efficiency of yCD2 protein from the viral envelope protein, and the proper processing of the viral envelope protein, cell lysates were generated from infected U87-MG cells. U87-MG cells at maximal infectivity, confluent cell monolayer was washed once in 1×PBS, dissociated by TrpZean (Sigma), resuspended in complete DMEM, washed again in 1×PBS, followed by cell lysis in 200 μL of RIPA lysis buffer (Thermo Scientific) on ice for 30 minutes. The lysates were clarified of cellular debris by centrifugation at 14,000 rpm for 15 minutes at 4° C. and the supernatants collected and transferred to a new tube. The cell lysates were then assayed for their protein concentration using BCA precipitation assay (Thermo Scientific) and 20 μg protein was subjected to SDS-PAGE. The proteins were resolved on 4-12% XT-Tris SDS-PAGE gels (BioRad) for 45 minutes at 200 volts. Subsequently the proteins were transferred onto PVDF membranes (Life Technologies) using an iBlot dry blotting system at 20 volts for 7 minutes. The membranes were assayed for the expression of the gp70 subunit of the envelope protein and the yCD2, using anti-gp70 (rat anti-gp70, clone 83A25; 1:500 dilution) and anti-yCD2 (mouse anti-yCD2; 1:1000 dilution). Protein expression was detected using the corresponding secondary antibody conjugated to horseradish peroxidase. The result show that yCD2 protein from RRV-P2A-yCD2 and RRV-T2A-yCD2 were separated inefficiently from the viral envelope protein, as indicated by the high molecular weight of the env-2A-yCD2 fusion polyprotein at ˜110 KDa, using the anti-yCD2 antibody. In contrast, the separation of yCD2 protein from the viral envelope protein was relative efficient for RRV-GSG-P2A-yCD2 and RRV-GSG-T2A-yCD2 compared to that from RRV-IRES-yCD2. In parallel, the processing of the viral envelope protein in infected U87-MG was examined using the anti-gp70 antibody. The result showed the viral enveloped in either precursor (Pr85) or processed form (gp70) were readily detectable in RRV-GSG-P2A-yCD2, RRV-GSG-T2A-yCD2 vector, but at much lower level in RRV-P2A-yCD2 and RRV-T2A-yCD2 vectors. In addition, the level of Pr85/gp70 viral envelope protein is somewhat consistent with that observed in the anti-yCD2 immunblot. However, unlike RRV-2A-GFPm or RRV-GSG-2A-GFPm vectors, viral envelope-yCD2 fusion polyprotein could not be detected using the anti-gp70 antibody or anti-2A antibody (Cat #ABS31, EMD Millipore). Among the 4 vectors, RRV-GSG-P2A-yCD2 and RRV-GSG-T2A-yCD2 vectors showed most efficient separation of fusion polyprotein as indicated by the lack of detection of the viral envelope-yCD2 fusion polyprotein in the anti-yCD2 immunoblot. All together the data suggest that GSG-P2A and GSG-T2A configuration give rise to the most efficient polyprotein separation in the context of RRV envelope protein open reading frame.

### Example 9: RRV-G2G-P2A-YCD2 and RRV-GSG-T2A-yCD2 have Long-Term Stability in U87-MG Cells

Serial infection was performed to evaluate long-term vector stability of RRV-GSG-P2A-yCD2 and RRV-GSG-T2A-yCD2 in U87-MG cells. Approximately 10¹ naïve U87-MG cells seeded in 6-well plates were initially infected with the viral vectors at a MOI of 0.1 and cultured for 1 week to complete a single cycle of infection. 100 μL of the 2 ml of viral supernatant from fully infected cells is used to infect 10¹ naïve cells and repeated up to 16 cycles. The genomic DNA was extracted from the small pellet by resuspending in 400 μL 1×PBS and isolated using the Promega Maxwell 16 Cell DNA Purification Kit (Promega). One-hundred nanogram of genomic DNA was then use as the template for PCR with a primer pair that spans the transgene cassette; IRES-F (5′-CTGATCTTACTCTTTGGACCTTG-3′ (SEQ ID NO:23)) and IRES-R (5′-CCCCTTTTTCTGGAGACTAAATAA-3′ (SEQ ID NO:24)). Vector stability of the 2A-yCD2 region is evaluated by PCR amplification of the integrated provirus from the infected cells. The expected PCR product size is approximately 0.73 kb. The appearance of any bands smaller than 0.73 kb indicates deletion in the 2A-yCD2 region. IRES-yCD2 (1.2 Kb) region in RRV-yCD2 is stable up to infection cycle 16 as previously reported (Perez et al., 2012). Similary, 2A-yCD2 region in both RRV-GSG-P2A-yCD2 and RRV-GSG-T2A-yCD2 also remains stable up to infection cycle 16. However, 2A-yCD2 region in RRV-GSG-T2A-yCD2 is slightly less stable than RRV-GSG-P2A-yCD2 as deletion (0.4 kb) deletion emerged from infection cycle 13 but remains stable throughout cycle 16.

### Example 10: Incorporation of Properly Processed Viral Envelope Protein Correlates with the Efficiency of Separation Between the Viral Envelope and yCD2 Proteins in U87-MG Cells Infected with RRV-P2A-yCD2 and RRV-T2A-yCD2, RRV-GSG-P2A-yCD2 and RRV-GSG-T2A-YCD2 Vectors

Viral supernatants produced from RRV-2A-yCD2 and RRV-GSG-2A-yCD2 maximally infected U87-MG cells, were pelleted through a 20% sucrose gradient at 14,000 rpm for 30 minutes at 4° C., and subsequently resuspended in 20 uL of 1× Laemmli Buffer containing 5% 2-mercaptoethanol and subjected to SDS PAGE on 4-20% Tris Glycine gels (BioRad, Hercules Calif.). The electrophoresis and protein transfer were performed as described. Properly processed virion viral envelop protein expression and maturation was assayed for using anti-gp70 (rat raised anti-gp70, clone 83A25; 1:500 dilution) and anti-p15E (mouse raised anti-TM, clone 372; 1:250 dilution). Protein expression was detected using the corresponding secondary antibody conjugated to horseradish peroxidase. The data show that properly processed envelope protein, gp70 of RRV-GSG-P2A-yCD2 and RRV-GSG-T2A-yCD2, but not RRV-P2A-yCD2 and RRV-T2A-yCD2, were detected at levels comparable to that of RRV-IRES-yCD2 in virions.

Importantly, the data suggest that the level of incorporation of properly processed viral envelope protein does not correlate with titer values.

### Example 11: yCD2 Protein Expression Level Varied in RRV-P2A-yCD2 and RRV-T2A-yCD2, RRV-GSG-P2A-yCD2 and RRV-GSG-T2A-yCD2 Infected U87-MG Cells but Exhibited Comparable 5-FC Sensitivity to that of RRV-IRES-yCD2 Infected U87-MG Cells

As the immunoblots of RRV-P2A-yCD2 and RRV-T2A-yCD2, RRV-GSG-P2A-yCD2 and RRV-GSG-T2A-yCD2 showed that the amount of yCD2 protein expressed either as separated protein from the viral envelope protein or as a fusion polyprotein varied in infected U87-MG cells, their 5-FC sensitivity was measured by performing a LD₅₀ experiment. Maximally infected U87-MG cells with RRV-P2A-yCD2 and RRV-T2A-yCD2, RRV-GSG-P2A-yCD2 and RRV-GSG-T2A-yCD2 vectors were used to determine their 5-FC LD₅₀ by MTS assay. For each infected or non-infected U87-MG cell line, 1×10³ cells/well/100 μL culture media were seeded in triplicate in 96-well plates. Cells were treatmented with 5-FC (cat #F7129, Sigma) in a series of 1:10 dilutions ranging from 0.00001 mM-1 mM. No 5-FC treatment was included as a control. 5-FC was added 1 day after plating and then replenished with complete medium plus 5-FC every 2 days. Naïve U87-MG cells were included as a control to determine non-5-FU mediated cytotoxic effect of 5-FC. The cells were monitored over a 7-day incubation time, and cell death was measured every 2 days by using the CellTiter 96 AQueous One Solution Cell Proliferation Assay System (Promega). Following the addition of the MTS, OD value at 490 nm were acquired using the Infinite M200 (Tecan) plate reader at 60-minute post MTS incubation. Averaged OD values from triplicates of each sample were converted to percentage of cell survival relative to untreated, but RRV-infected cells. Subsequently, the percentage values were plotted against 5-FC concentrations in log scale using GraphPad Prim to generate LD50 graphs. LD₅₀ values were calculated by the software using nonlinear four-parameter fit of the data points acquired. The data indicate that although the level of “separated” yCD2 protein were higher in RRV-GSG-P2A-yCD2 and RRV-GSG-T2A-yCD2 infected U87-MG cells than RRV-P2A-yCD2 and RRV-T2A-yCD2 infected U87-MG cells, the viral envelope-yCD2 fusion polyprotein observed in RRV-P2A-yCD2 and RRV-T2A-yCD2 infected U87-MG cells are enzymatically active in converting 5-FC to 5-FU to achieve cytotoxicitic effect at a LC₅₀ concentration similar to that of RRV-IRES-yCD2.

### Example 12: RRV-GSG-P2A-yCD2 and RRV-GSG-T2A-yCD2 Infected Tu2449 Cells Exhibited Comparable 5-FC Sensitivity to that of RRV-IRES-yCD2

Maximally infected Tu2449 cells with RRV-GSG-P2A-GMCSF-T2A-yCD2 was used to determine its 5-FC LD50 by MTS assay as described. RRV-IRES-yCD2 was included as a control. Treatment with 5-FC (cat #F7129, Sigma) in a series of 1:10 dilutions ranging from 0.00001 mM-1 mM was used. No 5-FC treatment was included as a control. 5-FC was added 1 day after plating and then replenished with complete medium plus 5-FC every 2 days. Naïve Tu2449 cells were included as a control to determine non-5-FU mediated cytotoxic effect of 5-FC. The cells were monitored over a 7-day incubation time, and cell death was measured every 2 days by using the CellTiter 96 AQueous One Solution Cell Proliferation Assay System (Promega). Following the addition of the MTS, OD value at 490 nm were acquired using the Infinite M200 (Tecan) plate reader at 60-minute post MTS incubation. Averaged OD values from triplicates of each sample were converted to percentage of cell survival relative to untreated, but RRV-infected cells. The percentage values were plotted against 5-FC concentrations in log scale using GraphPad Prim to generate LD₅₀ graphs. LD₅₀ values were calculated by the software using nonlinear four-parameter fit of the data points acquired. The data indicate that yCD2 protein expressed by RRV-GSG-P2A-yCD2 and RRV-GSG-T2A-yCD2 infected Tu-2449 cells are enzymatically active in converting 5-FC to 5-FU to achieve cytotoxicitic effect at a LC₅₀ concentration similar to that of RRV-IRES-yCD2.

### Example 13: Subcutaneous, Syngeneic Glioma Mice Treated RRV-GSG-T2A-yCD2 Showed Delayed Tumor Growth Comparable to that of RRV-IRES-yCD2

The syngeneic cell line Tu-2449 was used as an orthotopic brain tumor model in B6C3F1 mice (Ostertag et al., 2012). A subline of Tu-2449 cells (Tu-2449SQ) was established for subcutaneous tumor modeling. A mixture of 98z naïve Tu-2449 SQ cells and 2% RRV-GSG-T2A-yCD2 infected Tu-2449SQ cells were prepared in vitrol and resuspended in phosphate-buffered saline (PBS; Hyclone) for subcutaneous tumor implantation. A mixture of 98 naïve Tu-2449SQ cells and 2? RRV-IRES-yCD2 infected Tu-2449SQ cells was incluced as a positive control as well as a comparator. B6C3F1 mice in each group (n=10 per group) undergo subcutaneous implantation of 1×10⁶ tumor cells on day 0. On day 12 post tumor implant (at the time approximately >75? of tumors are infected with RRV), mice are administered with either PBS or 5-FC (500 mg per kg body weight per dose, i.p., b.i.d.) for 45 consecutive days, followed by 2 days without drug to allow vector spread from the remaining infected cells. Cycles of 5-day on, 2-day off drug treatment were repeated two additional times. The tumor volumetric measurement was taken daily. The results indicate that mice bearing tumor carrying RRV-IRES-yCD2 or RRV-GSG-T2A without 5-FC treatment continue to grow. In contrast, mice bearing tumor carrying RRV-GSG-T2A followed by 5-FC treatment delayed tumor growth of pre-established tumor and is comparable to that treated with RRV-IRES-yCD2+5-FC. The data suggest that in subcutaneous, syngeneic glioma mouse model, RRV-GSG-T2A-yCD2 have comparable therapeutic efficacy as RRV-IRES-yCD2.

### Example 14: RRV-GSG-T2A-GMCSF-GSG-P2A-yCD2 and RRV-GSG-T2A-yCD2-GSG-PS2-GMCSF Vectors Produced from HEK293T Cells Express GMCSF and yCD2 Proteins and are Infectious

pAC3-GSG-T2A-GMCSF-GSG-P2A-yCD2 and RRV-GSG-T2A-yCD2-GSG-P2A-GMCSF were generated by cloning of the human GMCSF-GSG-P2A-yCD2 and yCD2-GSG-P2A-GMCSF cassette chemically synthesized (Genewiz) with AscI and NotI restriction site present at the 5′ and 3′ end, respectively, into pAC3-GSG-T2A-yCD2 backbone digested with AscI and NotI restriction enzymes. The resultant GMCSF-GSG-P2A-yCD2 and yCD2-GSG-P2A-GMCSF cassette are in-frame with GSG-T2A at the N-terminus (5′ upstream of the AscI restriction site) of the cassete.

HEK293T cells were seeded at 2e6 cells per 10-cm plates, 18 to 20 hours pre transfection. The next day, 20 μg of pAC3-GSG-T2A-GMCSF-GSG-P2A-yCD2 ro pAC3-GSG-T2A-yCD2-GSG-P2A-GMCSF plasmid was used for transient tranfection at 20 hours post-cell seeding using the calcium phosphate method. Eighteen hours post-transfection, cells were washed with CMEM medium three times and incubated with fresh complete medium. Viral supernatant was collected approximately 42 hours post-transfection and filtered through a 0.45 μm syringe filter. The viral titers of RRV-GSG-T2A-GMCSF-GSG-P2A-yCD2 from transient transfection of HEK293T cells was determined as described. The data show that titers of RRV-GSG-T2A-GMCSF-GSG-P2A-yCD2 and pAC3-GSG-T2A-yCD2-GSG-P2A-GMCSF (˜ 2E6 TU/mL) are comparable to that of RRV-IRES-yCD2.

To assess the yCD2 protein expression, cell lysates were generated from pAC3-GSG-P2A-GMCSF-GSG-T2A-yCD2 or pAC3-GSG-T2A-yCD2-GSG-P2A-GMCSF transiently tranfected 293T cells. In this experiment, pAC3-IRES-yCD2 and pAC3-IRES-GMCSF were also included as controls. For GMCSF expression, supernatants transiently transfected 293T cells were collected for measurement by ELISA (Cat #DGM00, R & D Systems). The whole cell lysates were assayed for yCD2 protein expression as described. The anti-yCD2 result shows that yCD2 protein from pAC3-GSG-P2A-GMCSF-GSG-T2A-yCD2 or pAC3-GSG-T2A-yCD2-GSG-P2A-GMCSF is separated efficiently from the GMCSF, as indicated by the ˜15 KDa band. However, the separation of the yCD2 from GMCSF (pAC3-GSG-P2A-GMCSF-GSG-T2A-yCD2) or from viral envelope protein (pAC3-GSG-T2A-yCD2-GSG-P2A-GMCSF) mediated by the 2A peptide in both configurations are remarkably different, with proper separation of yCD2 protein from GMCSF as indicated by the size of yCD2 in comparison to yCD2 from RRV-IRES-yCD2. In contrast, yCD2 protein separation from the viral env has slightly higher molecular weight and is consistent with that of RRV-GSG-P2A-GFP, RRV-GSG-T2A-GFP, RRV-GSG-P2A-yCD2 and RRV-GSG-T2A-yCD2 constructs. The data suggest that the yCD2 separation from the Env may not occur precisely at the theorectically expected amino acid sequence. But when yCD2 is placed downstream of another secreted protein (i.e. GMCSF), proper separation of yCD2 protein is observed. However, it is important to note that the enzymatic activity of 2A-yCD2 protein expressed from RRV-GSG-P2A-yCD2 and RRV-GSG-T2A-yCD2 appear not to affect the 5-FC sensitivity and cytotoxic effect both in vitro and in vivo.

Although the separation efficiency of GMCSF protein from the viral envelope protein in pAC3-GSG-P2A-GMCSF-GSG-T2A-yCD2 construct or from yCD2 in pAC3-GSG-T2A-yCD2-GSG-P2A-GMCSF construct is undetermined, GMCSF ELISA results indicate that the amount of secreted GMCSF is ˜500 ng/mL for RRV-GSG-P2A-GMCSF-GSG-T2A-yCD2 and ˜760 ng/mL for RRV-GSG-T2A-yCD2-GSG-P2A-GMCSF. In both cases, the amount of GMCSF expressed is about 20- to 30-fold more than that of RRV-IRES-GMCSF (25 ng/mL). In parallel, the processing of the viral envelope protein in infected U87-MG is examined using the anti-gp70 antibody. The result shows that the viral envelope protein in either the precursor (Pr85) or processed form (gp70) is readily detectable. Together the data suggest that both Env-GSG-T2A-GMCSF-GSG-P2A-yCD2 and Env-GSG-T2A-yCD2-GSG-P2A-GMCSF polyprotein configurations can express GMCSF and yCD2 proteins.

In addition, viral supernatants collected from maximally infected U87-MG cells are titered as described to ensure the virus remain infectious. The data show that titers (˜3E6 TU/mL) produced from maximally infected U87-MG cells are similar to those obtained from transiently transfected HEK293T cells and are comparable to RRV-IRES-yCD2.

### Example 15: RRV-GSG-T2A-GMCSF-P2A-yCD2 and RRV-GSG-T2A-yCD2-P2A-GMCSF Vectors Exhibit Comparable 5-FC Sensitivity to that of RRV-IRES-yCD2 Infected U87-MG Cells

Maximally infected U87-MG cells with RRV-GSG-T2A-GMCSF-GSG-P2A-yCD2 or RRV-GSG-T2A-yCD2-GSG-P2A-GMCSF are used to determine its 5-FC LD₅₀ by MTS assay as described. RRV-IRES-yCD2 is included as a control. The data indicate that the amount of “separated” yCD2 protein detected in infected U87-MG cells is able to achieve cytotoxic effect at a LD₅₀ concentration of 0.008 mM, which is similar to that of RRV-IRES-yCD2.

### Example 16: RRV-GSG-T2A-GMCSF-RSV-yCD2 and Vector Produced from HEK293T Cells and Maximally Infected U87-MG Cells is Infectious and Express GMCSF and yCD2 Proteins

pAC3-GSG-T2A-GMCSF-RSV-yCD2 is generated by cloning of the human GMCSF-RSV-yCD2 cassette chemically synthesized (Genewiz) with AscI and NotI restriction site present at the 5′ and 3′ end, respectively, into pAC3-GSG-T2A-yCD2 backbone digested AscI and NotI restriction enzymes. The chemically synthesized GMCSF-RSV-yCD2 cassette contains a stop codon at the 3′ end of GMCSF ORF.

HEK293T cells are seeded at 2e6 cells per 10-cm plates, 18 to 20 hours pre transfection. The next day, 20 μg of pAC3-GSG-T2A-GMCSF-RSV-yCD2 plasmid is used for transient transfection at 20 h post-cell seeding using the calcium phosphate method. Eighteen hours post transfection, cells were washed with DMEM medium three times and incubated with fresh complete culture medium. Viral supernatant was collected approximately 42 h post-transfection and filtered through a 0.45 μm syringe filter. The viral titers of RRV-GSG-T2A-GMCSF-RSV-yCD2 from transient transfection of HEK293T cells is determined as described. The data show that titer of RRV-GSG-T2A-GMCSF-RSV-yCD2 (˜2E6 TU/mL) is comparable to that of RRV-IRES-yCD2.

In addition, viral supernatants collected from maximally infected U87-MG cells is titered to ensure the virus remains infectious. The data show that titer (˜2E6 TU/mL) produced from maximally infected U87-MG cells is similar to those obtained from transiently transfected HEK293T cells and is comparable to RRV-IRES-yCD2.

To assess the GMCSF and yCD2 protein expression, cell lysates are generated from RRV-GSG-T2A-GMCSF-RSV-yCD2 infected U87-MG cells. In this experiment, RRV-IRES-yCD2 and RRV-IRES-GMCSF are included as controls. Supernatant from maximally infected U87-MG cells is collected for measuring the protein expression level of GMCSF by ELISA (R & D Systems). The whole cell lysates are assayed for yCD2 protein expression as described. The anti-yCD2 immunoblot result shows that yCD2 protein from RRV-GSG-T2A-GMCSF-RSV-yCD2 infected U87-MG cells is expressed at the level ˜2-3 times less than that of RRV-IRES-yCD2. In parallel, the processing of the viral envelope protein in infected U87-MG is examined using the anti-gp70 antibody. The result shows that the viral envelope protein in either precursor (Pr85) or processed form (gp70) is readily detectable. As expected, viral envelope-GMCSF fusion polyprotein is also detected in cell lysates using the anti-gp70 antibody. Although the separation of GMCSF protein from the viral envelope protein is undetermined, GMCSF ELISA result indicates that the amount of secreted GMCSF is ˜300 ng/mL and is about 10-fold more than that of RRV-IRES-GMCSF (30 ng/mL). Together the data suggest that viral envelop protein-GSG-T2A-GMCSF-RSV-yCD2 polyprotein configuration can produce infectious virus as well GMCSF and yCD2 protein in the context of RRV.

### Example 17: RRV-GSG-T2A-GMCSF-RSV-yCD2 Vector Exhibits Comparable 5-FC Sensitivity to that of RRV-IRES-yCD2 Infected U87-MG Cells

Maximally infected U87-MG cells with RRV-GSG-T2A-GMCSF-RSV-yCD2 vector is used to determine its 5-FC LD50 by MTS assay as described. In this experiment, RRV-IRES-yCD2 is included as a control. The data indicate that the amount of yCD2 protein expressed in infected U87-MG cells is able to achieve cytotoxicitic effect at a LD₅₀ concentration of 0.010 mM and is comparable to that of RRV-IRES-yCD2.

### Example 18: RRV-GSG-P2A-yCD2-RSV-PDL1miR30shRNA Vector Produced from 293T Cells and Infected U87-MG Cells is Infectious and Express yCD2 Protein

pAC3-GSG-T2A-yCD2-RSV-miRPDL1 is generated by cloning of the human yCD2-RSV-miRPDL1 cassette chemically synthesized (Genewiz) with AscI and NotI restriction site present at the 5′ and 3′ end, respectively, into pAC3-GSG-T2A-yCD2 backbone digested AscI and NotI restriction enzymes. The chemically synthesized yCD2-RSV-miRPDL1 cassette contains a stop codon at the end of yCD2 ORF.

HEK293T cells are seeded at 2e6 cells per 10-cm plates, 18 to 20 hours pre transfection. The next day, 20 μg of pAC3-GSG-T2A-yCD2-RSV-miRPDL1 plasmid is used for transient transfection at 20 h post-cell seeding using the calcium phosphate method. Eighteen hours post transfection, cells were washed with DMEM medium three times and incubated with fresh complete culture medium. Viral supernatant was collected approximately 42 h post-transfection and filtered through a 0.45 μm syringe filter. The viral titers of RRV-GSG-T2A-yCD2-RSV-mrRPDL1 from transient transfection of HEK293T cells is determined as described. The data show that titer of RRV-GSG-T2A-yCD2-RSV-miRPDL1 (˜2E6 TU/mL) is comparable to that of RRV-IRES-yCD2.

In addition, viral supernatants collected from maximally infected U87-MG cells is titered to ensure the virus remains infectious. The data show that titer (˜2E6 TU/mL) produced from maximally infected U87-MG cells is similar to those obtained from transiently transfected HEK293T cells and is comparable to RRV-IRES-yCD2.

To measure the expression of yCD2 protein and PDL1 cell surface expression, maximally infected U87-MG cells are harvested and the whole cell lysates are assayed for yCD2 protein expression as described. The anti-yCD2 immunoblot result shows that yCD2 protein from RRV-GSG-T2A-yCD2-RSV-miRPDL1 infected U87-MG cells is separated efficiently from the viral envelope protein, as indicated by the ˜15 KDa band using the anti-yCD2 antibody. As expected, viral envelope-yCD2 fusion polyprotein is also detected in the cell lysates using both anti-yCD2 and anti-gp70 antibodies. In parallel, the processing of the viral envelope protein in infected U87-MG is examined using the anti-gp70 antibody. The result shows that the viral envelope protein in either precursor (Pr85) or processed form (gp70) is readily detectable. In addition, fusion polyproteins are detected as seen in the anti-yCD2 immmunoblot.

### Example 19: RRV-GSG-T2A-yCD2-RSV-miRPDL1 Infected U87-MG Cells Exhibits Comparable 5-FC Sensitivity to that of RRV-IRES-yCD2 Infected U87-MG Cells

Maximally infected U87-MG cells with RRV-GSG-T2A-yCD2-RSV-miRPDL1 vector is used to determine its 5-FC LD₅₀ by MTS assay as described. In this experiment RRV-IRES-yCD2 is included as a control. The data indicate that the amount of “separated” yCD2 protein detected in infected U87-MG cells is able to achieve cytotoxicitic effect at a LD₅₀ concentration (0.008 mM) comparable to that of RRV-IRES-yCD2.

### Example 20: RRV-GSG-P2A-yCD2-RSV-miRPDL1 Infected MDA-MB231 Cells Exhibits Potent PD-L1 Knockdown on the Cell Surface

To assess PDL1 knockdown activity of RRV-GSG-T2A-yCD2-RSV-miRPDL1, a MOI of 0.1 is used to infect MDA-MB231 cells which have been shown to express marked level of PDL1. In this experiment, RRV-RSV-miRPDL1 is included as a positive control for assessing PDL1 knockdown activity. Approximately at day 14 post infection, cells are harvested and cell surface staining is performed to measure the level of PDL1 protein by FACS. The data shows that the cell surface expression of PDL1 in MDA-MB231 cells infected with RRV-GSG-T2A-yCD2-RSV-miRPDL1 is decreased by approximately 75% and is comparable to that of RRV-RSV-miRPDL1. Together the data suggest that viral envelope protein-GSG-T2A-yCD2-RSV-miRPDL1 configuration can produce infectious virus, yCD2 protein and miRPDL1 in the context of RRV.

### Example 21: RRV-P2A-TKO RRV-GSG-P2A-TKO, RRV-T2A-TKO and RRV-GSG-T2A-TKO Vectors Produced from HEK293T Cells and Maximally Infected U87-MG Cells are Infectious and Express TKO Protein

pAC3-P2A-TKO, pAC3-GSG-P2A-TKO, pAC3-T2A-TKO and pAC3-GSG-T2A-TKO were generated by cloning of a Sr39-tk (Black et al., Cancer Res., 61:3022-3026, 2001; Kokoris et al., Protein Science 11:2267-2272, 2002) with human codon optimization (TKO), (see, International Application Publ. No. WO2014/066700, incorporated herein by reference) cassette into pAC3-2A backbone. Sequence of TKO was chemically synthesized (Genewiz) with AscI and NotI restriction site present at the 5′ and 3′ end, respectively, into pAC3-GSG-P2A-yCD2 or pAC3-GSG-T2A-yCD2 backbone digested with AscI and NotI restriction enzymes.

HEK293T cells were seeded at 2e6 cells per 10-cm plates, 18 to 20 hours pre transfection. The next day, 20 μg of pAC3-GSG-P2A-TKO or pAC3-GSG-T2A-TKO plasmid was used for transient transfection at 20 h post-cell seeding using the calcium phosphate method. Eighteen hours post transfection, cells were washed with DMEM medium three times and incubated with fresh complete medium. Viral supernatant was collected approximately 42 h post-transfection and filtered through a 0.45 μm syringe filter. The viral titers of RRV-P2A-TKO, RRV-GSG-P2A-TKO, RRV-T2A-TKO and RRV-GSG-T2A-TKO from transient transfection of HEK293T cells was determined as described. The data show that titers are comparable to that of RRV-IRES-yCD2 (Table G).

TABLE G
Titer of RRV-P2A-TKO RRV-GSG-P2A-TKO, RRV-T2A-TKO and
RRV-GSG-T2A-TKO vectors produced from HER293T cells

| | | | Titer of qPCR replicates (TU/mL) | | | Mean of dilution reps | | |
|---|---|---|---|---|---|---|---|---|
| | Sample Titered | dilution | Well 1 | Well 2 | Well 3 | Trans rep | Std Dev | CV (%) |
| 5 | RRV-RSV-GFP | 1 | 7.90E+05 | 6.97E+05 | 8.71E+05 | 8.05E+05 | 1.03E+05 | 12.80% |
| 6 | RRV-RSV-GFP | 1 | 8.42E+05 | 6.81E+05 | 9.47E+05 | | | |
| 7 | RRV-RSV-TKO | 1 | 4. [symbol] 5E+05 | 5.63E+05 | 4.91E+05 | 4.97E+05 | 4.29E+04 | 8.63% |
| 8 | RRV-RSV-TKO | 1 | 5.13E+05 | 4.31E+05 | 4. [symbol] E+05 | | | |
| 9 | RRV-P2A-TKO | 1 | 1.14E+06 | 1.26E+06 | 1.28E+06 | 1.12E+06 | 1.59E+05 | 14.23% |
| 10 | RRV-P2A-TKO | 1 | 1.16E+06 | 8.69E+05 | 1.00E+06 | | | |
| 11 | RRV-GSG-P2A-TKO | 1 | 1.03E+06 | 9.75E+05 | 9.84E+05 | 1.07E+06 | 8.40E+04 | 7.85% |
| 12 | RRV-GSG-P2A-TKO | 1 | 1.18E+06 | 1.14E+06 | 1.12E+06 | | | |
| 13 | RRV-T2A-TKO | 1 | 9. [symbol] 1E+05 | 1.09E+06 | 1.07E+06 | 1.15E+06 | 1. [symbol] 4E+05 | 11. [symbol] % |
| 14 | RRV-T2A-TKO | 1 | 1.28E+06 | 1.21E+06 | 1.29E+06 | | | |
| 15 | RRV-GSG-T2A-TKO | 1 | 1.17E+06 | [symbol] E+06 | 1.3 [symbol] E+06 | 1.53E+06 | 2.42E+05 | 15.78% |
| 16 | RRV-GSG-T2A-TKO | 1 | 1.62E+06 | 1.88E+06 | 1. [symbol] 0E+06 | | | |
| 17 | RRV-GSG-T2A-GFP | 1 | [symbol] .16E+06 | 1. [symbol] 0E+06 | 1.4 [symbol] E+06 | 1.65E+06 | 3.09E+05 | 18.70% |
| 18 | RRV-GSG-T2A-GFP | 1 | 1.73E+06 | 1.38E+06 | 1.4 [symbol] E+06 | | | |
| 19 | RRV-IRES-GFP | 1 | 8.12E+05 | 9.68E+05 | 7.31E+05 | 7.73E+05 | 1.18E+05 | 15.25% |
| 20 | RRV-IRES-GFP | 1 | 7.73E+05 | 7.45E+05 | [symbol] .07E+05 | | | |
| 21 | Mock 293T Sup | 1 | #VALUE! | #VALUE! | #VALUE! | #VALUE! | #VALUE! | #VALUE! |
| 22 | Mock 293T Sup | 1 | 5.17E+06 | #VALUE! | #VALUE! | | | |
| 23 | [symbol] TGOT0 [symbol] 7 (Exp 273) | 200 | 2.38E+08 | 1.66E+08 | 1.64E+08 | 1.93E+08 | 4.11E+07 | 21.32% |
| 24 | [symbol] TGOT0 [symbol] 7 (Exp 273) | 200 | 2.53E+08 | 1.70E+08 | 1. [symbol] E+08 | | | |

[symbol]

indicates data missing or illegible when filed

In addition, viral supernatants collected from maximally infected U87-MG cells is titered as described to ensure the virus remain infectious. The data show that titers produced from maximally infected U87-MG cells are comparable to those obtained from transiently transfected HEK293T cells.

To assess the TKO protein expression, cell lysates were generated from RRV-P2A-TKO RRV-GSG-P2A-TKO, RRV-T2A-TKO and RRV-GSG-T2A-TKO infected U87-MG cells. The whole cell lysates were assayed for TKO protein expression using anti-HSV-tk antibody (Cat #sc28037, Santa Cruz Biotech Inc) at 1:200. The result shows that TKO protein from RRV-P2A-TKO and RRV-T2A-TKO infected U87-MG cells is separated less efficiently than RRV-GSG-P2A-TKO and RRV-GSG-T2A-TKO as seen previously with GFP and yCD2 transgenes.

### Example 22: RRV-P2A-TKO RRV-GSG-P2A-TKO, RRV-T2A-TKO and RRV-GSG-T2A-TKO Vectors are Stable in U87-MG Cells

To evaluate the vector stability in maximally infected U87-MG cells, genomic DNA was extracted from cells using the Promega Maxwell 16 Cell DNA Purification Kit (Promega). One-hundred nanogram of genomic DNA was then use as the template for PCR with a primer pair that spans the transgene cassette; IRES-F (5′-CTGATCTTACTCTTTGGACCTTG-3′ (SEQ ID NO:23)) and IRES-R (5′-CCCCTTTTTCTGGAGACTAAATAA-3′ (SEQ ID NO:24)) as previously described. The expected PCR product for all RRV-2A-TKO constructs is 1.4 kb. The data show that the 2A-TKO and GSG-2A-TKO region in proviral DNA RRV-P2A-TKO RRV-GSG-P2A-TKO, RRV-T2A-TKO and RRV-GSG-T2A-TKO vectors are stable in U87-MG cells during the time course of viral replication.

### Example 23: RRV-P2A-TKO, RRV-GSG-P2A-TKO, RRV-T2A-TKO and RRV-GSG-T2A-TKO Infected U87-MG Cells Exhibited Superior GCV Sensitivity to that of RRV-S1-TKO

Maximally infected U87-MG cells with RRV-P2A-TKO, RRV-GSG-P2A-TKO, RRV-T2A-TKO and RRV-GSG-T2A-TKO were used to determine its GCV LD₅₀ by MTS assay. RRV-S1-TKO of which the TKO expression driven by a synthetic minimal promoter (see, International Pat. Publ. No. WO2014/066700, incorporated herein by reference) was included as a control. Treatment with GCV (cat #345700-50 MG, EMD Millipore) was performed in a series of 1:2 dilutions ranging from 0.0001 μM-0.5 μM. No GCV treatment was included as a control. GCV was added 1 day after plating and then replenished with complete medium plus GCV every 2 days. Naïve U87-MG cells were included as a control to determine cytotoxic effect of GCV. The cells were monitored over a 7-day incubation time, and cell death was measured every 2 days by using the CellTiter 96 AQueous One Solution Cell Proliferation Assay System (Promega). Following the addition of the MTS, OD value at 490 nm were acquired using the Infinite M200 (Tecan) plate reader at 60-minute post MTS incubation. Averaged OD values from triplicates of each sample were converted to percentage of cell survival relative to untreated, but RRV-infected cells. The percentage values were plotted against GCV concentrations in log scale using GraphPad Prim to generate LD₅₀ graphs. LD₅₀ values were calculated by the software using nonlinear four-parameter fit of the data points acquired. The data indicate that the TKO protein expressed by RRV-P2A-TKO, RRV-GSG-P2A-TKO, RRV-T2A-TKO and RRV-GSG-T2A-TKO is enzymatically active in converting GCV to cytotoxic GCV at tenth of millimolar range to achieve cytotoxicitic effect. In comparison to RRV-S1-TKO, RRV-P2A-TKO, RRV-GSG-P2A-TKO, RRV-T2A-TKO and RRV-GSG-T2A-TKO show 12.5-20-fold higher GCV sensitivity. In addition, there was no significant difference in GCV LD50 between RRV-P2A-TKO vs RRV-GSG-P2A-TKO or RRV-T2A-TKO vs RRV-GSG-T2A-TKO despite the difference in TKO separation from the Env-TKO fusion polyprotein. Similar to 2A-yCD2, the data suggest that the amount of TKO protein expressed in the cells is sufficient to convert GCV to cytotoxic GCV.

### Example 24: Subcutaneous, Syngeneic Glioma Mice Treated RRV-GSG-P2A-TKO and RRV-GSG-T2A-TKO Show Delayed Tumor Growth Comparable to that of RRV-IRES-yCD2

The syngeneic cell line Tu-2449 was used as an orthotopic brain tumor model in B6C3F1 mice (Ostertag et al., 2012). A subline of Tu-2449 cells (Tu-2449SQ) was established at Tocagen for subcutaneous tumor model. A mixture of 98V naïve Tu-2449SQ cells and 2% RRV-GSG-P2A-TKO, RRV-GSG-T2A-TKO or RRV-S1-TKO infected Tu-2449SQ cells were prepared in vitro and resuspended in phosphate-buffered saline (PBS; Hyclone) for subcutaneous tumor implantation. A mixture of 98% naïve Tu-2449SQ cells and 2% RRV-IRES-yCD2 infected Tu-2449SQ cells was included as a positive control as well as a comparator. B6C3F1 mice in each group (n=10 per group) undergo subcutaneous implantation of 1×10⁶ tumor cells on day 0. On day 12 post tumor implant (at the time approximately >75% of tumors are infected with RRV), mice are administered with either PBS, 5-FC (500 mg per kg body weight per dose, i.p., b.i.d.) or GCV (50 mg per kg body weight per dose, i.p., b.i.d.) for 5 consecutive days, followed by 2 days without drug to allow vector spread from the remaining infected cells. Cycles of 5-day on, 2-day off drug treatment were repeated two additional times. The tumor volumetric measurement was taken daily. The results indicate that mice bearing tumor carrying RRV-GSG-P2A-TKO, RRV-GSG-T2A-TKO or RRV-S1-TKO without GCV or RRV-IRES-yCD2 without 5-FC treatment continue to grow. In contrast, mice bearing tumor treated RRV-GSG-P2A-TKO, RRV-GSG-T2A-TKO+GCV delay tumor growth of pre-established tumor. Furthermore, mice breaing tumor treated with RRV-S1-TKO+GCV also shows delay in tumor growth although at lesser extent and longer time than tumor treated RRV-GSG-P2A-TKO, RRV-GSG-T2A-TKO+GCV, possibly due reduced TKO expression. Together, the data indicate that the delay in tumor growth of RRV-GSG-P2A-TKO+GCV and RRV-GSG-T2A-TKO+GCV is comparable to that treated with RRV-IRES-yCD2+5-FC. The data suggest that in subcutaneous syngeneic glioma mouse model, RRV-GSG-P2A-TKO and RRV-GSG-T2A-TKO have comparable therapeutic efficacy as RRV-IRES-yCD2.

### Example 25: RRV-GSG-T2A-PDL1scFv and RRV-GSG-T2A-PDL1scFvFc Vectors Produced from HEK293T Cells and Maximally Infected U87-MG Cells are Infectious and Express scFv and scFvFc Protein

pAC3-T2A-PDL1scFv, pAC3-T2A-PDL1scFv-Tag, pAC3-T2A-PDL1scFvFc and pAC3-T2A-PDL1scFvFc-Tag were generated to function as a blocking single chain variable fragment (scFv) against human and mouse PDL1. The PDL1scFv cassettes are designed with or without the fragment crystallizable (Fc) region of human IgG₁. In addition, the matching cassettes with HA and Flag epitope tags incorporated at the C-terminus of the scFv or ScFvFc were also generated for detection of scFv or scFvFc protein expression. Sequence of each cassettes (PDL1scFv,PDL1scFv-Tag, PDL1scFvFc and PDL1scFvFC-Tag) was chemically synthesized (Genewiz) with AscI and NotI restriction site present at the 5′ and 3′ end, respectively, and cloned into pAC3-GSG-T2A-yCD2 backbone digested with AscI and NotI restriction enzymes.

HEK293T cells were seeded at 2e6 cells per 10-cm plates, 18 to 20 hours pre transfection. The next day, 20 μg of pAC3-T2A-PDL1scFv, pAC3-T2A-PDL1scFv-Tag, pAC3-T2A-PDL1scFvFc and pAC3-T2A-PDL1scFvFc-Tag plasmid were used for transient transfection at 20 h post-cell seeding using the calcium phosphate method. Eighteen hours post transfection, cells were washed with DMEM medium three times and incubated with fresh complete medium. Viral supernatant was collected approximately 42 h post-transfection and filtered through a 0.45 μm syringe filter. The viral titers of RRV-GSG-T2A-GMCSF-GSG-P2A-yCD2 from transient transfection of HEK293T cells was determined as described. The data show that titer values of RRV-GSG-T2A-PDL1scFv, RRV-GSG-T2A-PDL1scFvFc, RRV-GSG-T2A-PDL1scFv-Tag, RRV-GSG-T2A-PDL1scFvFc-Tag are comparable to that of RRV-IRES-yCD2 (Table H).

TABLE H
Titer values of RRV-GSG-T2A-PDL1scFv, RRV-
GSG-T2A-PDL1scFvFc, RRV-GSG-T2A-PDL1scFv-
Tag, RRV-GSG-T2A-PDL1scFvFc-Tag from transiently
transfected HEK293T cells

| | TU/mL | Std Dev |
|---|---|---|
| RRV-PDL 1scFv | 2.09E+06 | 4.80E+05 |
| RRV-PDL 1scFv Fc | 1.98E+06 | 4.38E+05 |
| RRV-PDL 1scFv-Tag | 2.08E+06 | 6.73E+05 |
| RRV-PDL 1scFv Fc-Tag | 1.29E+06 | 1.87E+05 |

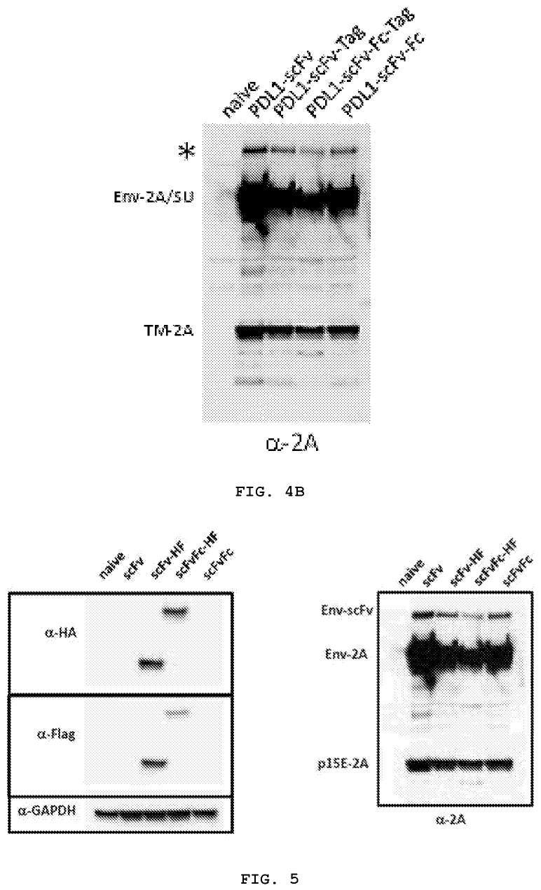

To evaluate the scFv protein expression, cell lysates were generated from RRV-GSG-T2A-PDL1scFv and RRV-GSG-T2A-PDL1scFvFc transfected HEK293T cells. The whole cell lysates were assayed for scFv protein expression using anti-Flag and anti-HA antibody (Cat #1804 and Cat #H3663, Sigma Aldrich) at 1:1,000. The result shows that PDL1scFv-Tag and PDL1scFvFc-Tag protein expression from RRV-GSG-T2A-PDL1scFv-Tag, RRV-GSG-T2A-PDL1scFvFc-Tag transiently transfected HEK293T cells are separated from the Env-scFv polyprotein (FIG. 4A) as seen previously with GFP and yCD2 and TKO transgenes.

In parallel, the processing of the viral envelope protein in HEK293T cells was examined using the anti-2A antibody. The result show the viral enveloped in either precursor (Pr85) or processed form (p15E) containing the 2A peptide sequence were detected in all 4 vectors (FIG. 4B), suggesting separation of the viral envelope protein from the scFv and scFvFc protein as seen in the anti-Flag and anti-HA immunoblots. Although fusion polyprotein, Env-scFv or Env-scFvFc, expression are detected in the cell lysates, significant amount of PDL1scFv and PDL1scFvFc proteinare separated from the fusion polyprotein as indicated by immunoblots from cell lysates and supernatant.

Similarly, abundant scFv-Tag and scFvFc-Tag protein expression are also detected in supernatant from transiently transfected HEK293T cells by immunoprecipitation with anti-Flag antibody followed by detection with anti-HA and vice versa. Furthermore, scFv-Tag and scFvFc-Tag protein expression cell lysates as well as supernatant are also detected from maxilly infected MDA-MB231 (human breast cancer cell line) and CT-26 (murine colorectal cancer cell line) cells at the levels approximately 2-3 times less than that from transiently transfected HEK293T cells.

### Example 26: RRV-GSG-T2A-PDL1scFv and RRV-GSG-T2A-PDL1scFvFc Restore PHA-Stimulated T-Cell Activation and Shows Equivalence of PDL1 Blocking Antibody In Vitro

To determine if PDL1 blocking on tumor cells by RRV-GSG-T2A-PDL1scFv or RRV-GSG-T2A-PDL1scFvFc could alleviate PDL1-mediated T-cell suppression, we perform a PDL1-mediated trans-suppression co-culture experiment. Here, we evaluate if modulation of PDL1 expression on various tumor cell lines could alter PHA-stimulated activation of healthy donor PBMC as measured by intracellular expression of IFNγ or release of IFNγ into the supernatant. To eliminate the potential pleiotropic effects of IFNγ pre-treatment in the trans-suppression co-culture assay, we set up a co-culture system using the human breast cancer cell line MDA-MB-231, which has a high PDL1 basal cell surface expression level. To confirm the necessity of PDL1 engagement in this assay, anti-PDL1 blocking antibody is also included. PDL1⁺ tumor cells MDA-MB-231 cells in the presence of anti-PDL1 blocking antibody is unable to suppress CD8⁺ T-cell activation as indicated by the increased frequency of IFNγ+/CD8+ T cells. Similarly, MDA-MB-231 cells infected with RRV-GSG-T2A-scFv or RRV-GSG-T2A-scFvFc equally restored CD8⁺ T-cell activation. The data indicate that disruption of the PDL1:PD1 axis on tumor cells and lymphocytes by PDL1 blocking scFv show comparable activity as anti-PDL1 blocking antibody and provides evidence for a substantial immunological benefit from RRV-GSG-T2A-PDL1scFv and RRV-GSG-T2A-PDL1scFvFc.

### Example 27: RRV, TOCA-511, Mutation Profiling

Various tumor types are variably able to support rapid RRV replication, and this variability can alter the susceptibility of different tumors to RRV based therapeutic treatment such as for the RRV Toca 511 (aka T5.0002) and prodrug Toca FC treatment for high grade glioma (T. F. Cloughsey et al., Sci Transl Med., 8(341):341ra75, Jun. 1, 2016, doi: 10.1126/scitranslmed.aad9784.) This variability is attributable to various factors but one that appears relevant, from our sequencing data of RRV encoding a modified yeast cytosine deaminase that have been recovered from patients' blood or tumor, is modification by the APOBEC function, particularly APOBEC3B and APOBEC3G (B. P. Doehle et al., J. Virol. 79: 8201-8207, 2005). Modification of expression is deduced from the frequency with which inactivating or attenuating mutations accumulate in the replicating retroviral vector as it progressively replicates in tumor tissue. Investigation shows that one of the most frequent events is G to A mutations, which corresponds to the C to T transition characteristic of APOBEC mediated mutations on the negative strand single stranded DNA from the first replicative step in the reverse transcription step. These mutations can cause changes in amino acid composition of the RRV proteins, for instance a devastating change from TGG (Tryptophan) to stop codons (TAG, TGA or TAA). It has been shown that some tumors (in particular bladder, cervix, lung (adenocarcinoma and squamous cell carcinoma), head and neck, and breast cancers, APOBEC3B activity is upregulated, and this upregulation correlates with increased mutational load with changes that are consistent with APOBEC3B activity (M B. Burns et al., Nature Genetics 45: 977-83, 2013; doi: 10.1038/ng.2701). The driver behind this upregulation is proposed to be that the higher mutational rate favors tumor evolution and selection for a tumor advantageous genotype and phenotype. In one embodiment, the inactivating change in the virus is avoided by substitution of codons for other amino acids with similar chemical or structural properties such as phenylalanine or tyrosine that will not be converted by APOBEC. Toca 511 is an MLV derived RRV that encodes a thermostable codon optimized yeast cytosine deaminase linked to an IRES, which catalyzes conversion of prodrug 5-FC to cytotoxic 5-FU. In the course of Toca 511 treatment, Toca 511 is susceptible to mutations, due to errors in reverse transcription and cellular anti-viral defense mechanisms such as APOBEC-mediated cytidine deaminase. APOBEC proteins target single stranded DNA, primarily during reverse transcription of Toca 511 RNA genome, manifesting as G to A point.

Toca 511 sequence mutation spectrum was profiled by high throughput sequencing of Toca 511 from clinical samples isolated from tumor and blood. G to A point mutation is the most common mutation type in Toca 511, consistent with APOBEC activity. This is the first characterization of gamma-retroviral gene therapy mutation spectrum from human samples via high throughput sequencing. An analysis of the G to A mutations shows that these usually lead to nonsynonymous changes in coding sequences. Within the gene encoding the cytosine deaminase polypeptide there were two positions with recurrent G to A mutations in samples from multiple patients (Table I). These mutations convert codon TGG encoding tryptophan to TGA, TAG or TAA stop codons and thus terminate CD translation after only nine amino acids. These results highlight that tryptophan codons are a potential source of inactivation of retroviral gene therapies.

TABLE I
Summary of point mutations in recombinant cytosine deaminase (SEQ
ID NO: 28-29) of Toca 511. Position is the amino acid position within
the CD protein. Samples indicated the number of clinical samples
from blood or tumor that showed mutation. Codon and change show
the original codon sequence and the subsequent change. AA is the
original amino acid encoded by the original codon and change shows
what the amino acid is changed to after the codon mutation.

| nucleotide | position | samples | codon | change | AA | change |
|---|---|---|---|---|---|---|
| 29 | 10 | 17 | TGG | TAG | W | STOP |
| 30 | 10 | 5 | TGG | TGA | W | STOP |
| 31 | 11 | 1 | GAT | AAT | D | N |
| 40 | 14 | 1 | GGC | AGC | G | S |
| 45 | 15 | 1 | ATG | ATA | M | I |
| 105 | 35 | 2 | GGC | GAC | G | D |
| 144 | 48 | 1 | AGG | AAG | R | K |
| 159 | 53 | 1 | AGG | AAG | R | K |
| 168 | 56 | 6 | AAG | AAA | R | K |
| 216 | 72 | 1 | GGC | GAC | G | S |
| 357 | 119 | 1 | GAG | AAG | E | K |
| 456 | 152 | 4 | TGG | TAG | Q | STOP |

Accordingly, changing tryptophan codons to alternative codons that encode amino acids compatible with protein function can mitigate APOBEC mediated inactivation of retroviral gene therapies.

To test the effects of mutations on stability, Toca 511 genome sequence (see, e.g., U.S. Pat. No. 8,722,867, SEQ ID Nos: 19, 20 and 22 of the '867 patent, which are incorporated herein by reference) is engineered to change the codons that that show ApoBec hyperumuation to codons that encode an alternative amino acid that preserves stability and function (e.g., changing codons for tryptophan to some other permissible amino acid). The Toca 511 polypeptide having cytosine deaminase activity (see, SEQ ID NO:29) is closely related to naturally occurring fungal cytosine deaminase proteins and high resolution structures of such cytosine deaminases are available. Thus it is possible to utilize the combination of structural and multiple sequence alignments from phylogenetically diverse fungal CD proteins to identify potential amino acid substitutions that will not have adverse effects on biological function, for instance using ROSETTA, Provean, PSIpred or similar programs. A set of putative amino acid substitutions are then tested, by altering Toca 511 genome and measuring enzyme and biological activity, solubility, thermostability in solution as well as the ability to function in cell culture assays and mouse tumors models such as conversion of 5-FC to 5-FU, initiate cell death, and activate the immune response against tumors to achieve durable responses. A similar analysis can be used for GAG, POL and ENV sequence to modify such sequences to remove codon susceptible to ApoBec hypermuations.

### Example 28: APOBEC-Resistant yCD Viral Vectors are Therapeutic in an Intracranial Human Xenograft (T98G) in Nude Mice

An intracranial xenograft model using the T98G human glioma cell line that highly expresses APOBEC is established to test RRV vector spread and biodistribution as well as therapeutic efficacy of APOBEC-resistant RCR-vector mediated cytosine deaminase suicide gene therapy in a nude mouse host under high APOBEC activity conditions.

Following acclimation, mice are randomly assigned to one of 9 Treatment groups (see group description below). Eight groups undergo intracranial administration into the right striatum of 1×10⁵ T98G cells administered/mouse on Day 0. Group 9 mice are not implanted with tumor. At Day 5, mice are injected with Formulation Buffer only, T5.0002 (APOBEC-sensitive RRV expressing yCD; group 3) at 9×10⁵ TU/5 μl or an APOBEC-resistant RCR vector (T5.002A) at 9×10⁵ TU/5 μl, 9×10⁴ TU/5 μl, or 9×10³ TU/5 μl. Randomized 5-FC dosing is performed at 500 mg/kg/day, administered as a single IP injection, beginning on Day 19, or some group are given no 5-FC (Groups, 1, 4, 8). Mice receiving vector at mid-dose all receive 5-FC (i.e., No separate control group for this dose). 5-FC administration continues daily for 7 consecutive days followed by 15 days of no treatment. Cycles of drug plus rest are repeated up to 4 cycles. 10 mice from each group except group 8 are randomly assigned to the survival analysis category. The remaining mice are sacrificed according to a predetermined schedule.

Group Assignments and Dose Levels

| | | | | | | N per Analysis Category | |
|---|---|---|---|---|---|---|---|
| | Test | | | Drug | | (A) Survival | (B) Scheduled |
| Group | article | Volume | | TX | N | analysis | Sacrifice |
| 1 | Form | 5 | μl | none | 4 | | 4 before first |
| | buffer | | | | | | drug cycle |
| 2 | Form | 5 | μl | 5-FC | 10 | 10 | |
| | buffer | | | | | | |
| 3 | T5.0002 | 9e5/5 | μl | 5FC | 25 | 10 | 3 before start of |
| | | | | | | | each cycle, 15 |
| | | | | | | | total |
| 4 | T5.0002A | 9e5/5 | μl | PBS | 10 | 10 | |
| 5 | T5.0002A | 9e5/5 | μl | 5FC | 25 | 10 | 3 before start of |
| | | | | | | | each cycle, 15 |
| | | | | | | | total |
| 6 | T5.0002A | 9e4/5 | μl | 5FC | 10 | 10 | |
| 7 | T5.0002A | 9e3/5 | μl | 5FC | 25 | 10 | 3 before start of |
| | | | | | | | each cycle, 15 |
| | | | | | | | total |
| 8 | T5.0002A | 9e3/5 | μl | PBS | 10 | 10 | |

| | | | | |
|---|---|---|---|---|
| 9 NO | none | 5FC | 15 | 3 before start of |
| TUMOR | | | | each cycle, 15 |
| | | | | total |

| | | | |
|---|---|---|---|
| Total Number of Animals | 134 | 70 | 64 |

Intravenous dosing is performed via injection into the tail vein. Intraperitoneal dosing is performed via injection into the abdomen with care taken to avoid the bladder. For intracranial injection mice are anesthetized with isoflurane and positioned in a stereotaxic device with blunt ear bars. The skin is shaved and betadine is used to treat the scalp to prepare the surgical site. The animal is placed on a heating pad and a scalpel is used under sterile conditions to make a midline incision through the skin. Retraction of the skin and reflection of the fascia at the incision site will allow for visualization of the skull. A guide cannula with a 3 mm projection, fitted with a cap with a 3.5 mm projection, is inserted through a small burr hole in the skull and attached with dental cement and three small screws to the skull. After hardening of the cement, the skin is closed with sutures. The projected stereotaxic coordinates are AP=0.5-1.0 mm, ML=1.8-2.0 mm, DV=3.0 mm. Exact stereotaxic coordinates for the cohort of animals is determined in a pilot experiment (2-3 animals) by injecting dye and determining its location. The animals are monitored during anesthesia recovery. Analgesics, buprenorphine, is administered subcutaneously (SC) before the end of the procedure then buprenorphine is administered approximately every 12 hrs for up to 3 days. Animals are monitored on a daily basis. Cells or vector are intracranially infused through an injection cannula with a 3.5 mm projection inserted through the guide cannula. The rate is controlled with a syringe pump fitted with a Hamilton syringe and flexible tubing. For cell injection, 1 microliter of cells is delivered at a flow rate of 0.2 microliters per minute (5 minutes total). For vector injection, 5 microliters of vector is delivered at a flow rate Of 0.33 microliters per minute (15 minutes total).

APOBEC-resistant Vector is delivered and calculated as transforming units (TU) per gram of brain weight to the mice. Using such calculation the translation of dose can be calculated for other mammals including humans. APOBEC-resistant Vector shows an effective dose-response while vectors sensitive to APOBEC activity show a diminished effective response. The same experiment is conducted in U87 cell lines transfected with an expression vector for human APOBEC3G or APOBEC3B that express these proteins at least 3 fold above the U87 natural levels that are implanted in a xenograft model. These experiments show that the modified codon virus designed to be APOBEC-resistant has a replication and/or therapeutic response advantage in the U87 lines with increased APOBEC levels over the original RRV that is without codon modification for APOBEC resistance.

### Example 29: APOBEC-Resistant yCD Viral Vector is Therapeutic in a Syngeneic Mouse Model of Brain Cancer

Additional experiments to demonstrate the methods and compositions of the disclosure in a syngeneic animal model are performed.

An intracranial implant model using the CT26 colorectal cancer cell line stably transfected to produce murine APOBEC3 in syngeneic BALB/c mice is established to test APOBEC-resistant RRV vector spread and biodistribution as well as therapeutic efficacy of RRV-vector mediated cytosine deaminase suicide gene therapy and its immunological impact.

This study includes 129 animals, 0 Male, 119 Female and 10 contingency animals (10 Female). Following acclimation, mice are randomly assigned to one of 9 Treatment groups (see group description below). Eight groups undergo intracranial administration into the right striatum of 1×10⁴ APOBEC-expressing CT26 cells administered/mouse on Day 0. Group 9 mice are not implanted with tumor. At Day 4, mice are injected with Formulation Buffer only, control vector that is still sensitive to APOBEC (T5.0002) at 9×10⁵ TU/5 μl, or APOBEC-resistant vector (T5.0002A) at 9×10⁵ TU/5 μl, 9×10⁴ TU/5 μl, or 9×10³ TU/5 μl. Mice receiving no vector, or vector at 9×10⁵ TU/5 μl or 9×10³ TU/5 μl are randomized to receive 5-FC (500 mg/kg/BID), administered by IP injection, beginning on Day 13, or no 5-FC as indicated (PBS). Mice receiving vector at mid dose receive 5-FC (i.e., No separate control group for this dose). 5-FC administration continues daily for 7 consecutive days followed by 10 days of no treatment. Cycles of drug plus rest are repeated up to 4 cycles. 10 mice from each group except group 9 are randomly assigned to the survival analysis category. The remaining mice are sacrificed according to a predetermined schedule.

Naïve sentinel mice are co-housed with the scheduled sacrifice animals and taken down at the same time points to assess vector transmittal through shedding.

Group Assignments and Dose Levels

| | | | | | | N per Analysis Category | | |
|---|---|---|---|---|---|---|---|---|
| | Test | | | Drug | | (A) Survival | (B) Scheduled | (C) |
| Group | article | Volume | | TX | N | analysis | Sacrifice | Sentinels |
| 1 | Form | 5 | μl | PBS | 4 | | 4 before | |
| | buffer | | | | | | first drug | |
| | | | | | | | cycle | |
| 2 | Form | 5 | μl | 5FC | 10 | 10 | | |
| | buffer | | | | | | | |
| 3 | T5.0002A | 9E5/5 | μl | PBS | 10 | 10 | | |
| 4 | T5.0002 | 9E5/5 | μl | 5FC | 10 | 10 | 3 before | 1 before |
| | | | | | | | start of | start of |
| | | | | | | | each cycle, | each cycle, |
| | | | | | | | 15 total | 5 total |
| 5 | T5.0002A | 9E5/5 | μl | 5FC | 25 | 10 | 3 before | 1 before |
| | | | | | | | start of | start of |
| | | | | | | | each cycle, | each cycle, |
| | | | | | | | 15 total | 5 total |
| 6 | T5.0002A | 9E4/5 | μl | 5FC | 10 | 10 | | |
| 7 | T5.0002A | 9E3/5 | μl | 5FC | 25 | 10 | 3 before | 1 before |
| | | | | | | | start of | start of |
| | | | | | | | each cycle, | each cycle, |
| | | | | | | | 15 total | 5 total |
| 8 | T5.0002A | 9E3/5 | μl | PBS | 10 | 10 | | |

| | | | | |
|---|---|---|---|---|
| 9 NO | none | 5FC | 15 | 3 before |
| TUMOR | | | | start of |
| | | | | each cycle, |
| | | | | 15 total |

| | | | | |
|---|---|---|---|---|
| Total Number of Animals | 119 | 70 | 64 | 15 |

Intravenous dosing is performed via injection into the tail vein. Intraperitoneal dosing is performed via injection into the abdomen with care taken to avoid the bladder. For intracranial administration, mice with a guide cannula with a 3.2 mm projection implanted into the right striatum, and fitted with a cap with a 3.7 mm projection are used. The projected stereotaxic coordinates are AP=0.5-1.0 mm, ML=1.8-2.0 mm, DV=3.2 mm (from bregma). Cells or vector are intracranially infused through an injection cannula with a 3.7 mm projection inserted through the guide cannula. The rate is controlled with a syringe pump fitted with a Hamilton syringe and flexible tubing.

For cell injection, 1 microliter of cells is delivered at a flow rate of 0.2 microliter per minute (5 minutes total). For vector injection, 5 microliter of vector is delivered at a flow rate of 0.33 microliter per minute (15 minutes total).

Vector is delivered and calculated as transforming units (TU) per gram of brain weight to the mice. Using such calculation the translation of dose can be calculated for other mammals including humans. Results from this study will show that APOBEC-resistant virus spreads throughout tumor, maintains yCD integrity and is more effective at treating the tumor in combination with 5FC when compared to APOBEC-sensitive RRV. APOBEC-resistant RRV also does not horizontally spread to naïve cage mates.

As described above, an RRV contains a “2A cassette”. For example, SEQ ID NOs:2, 43-53 and 54 provide a general construct containing a 2A cassette. The cassette can be replaced with a number of different cassettes. For example, the following cassettes can be prepare and cloned into any one of SEQ ID NO:2, 43-53 or 54 vector backbone replacing the cassette in those particular constructs.

Using the methods and sequences provided herein a number of vectors were designed as follows:

(SEQ ID NO: 43)
pAC3-T2A-GFPm
(SEQ ID NO: 44)
pAC3-GSG-T2A-GFPm
(SEQ ID NO: 45)
pAC3-P2A-GFPm
(SEQ ID NO: 46)
pAC3-GSG-P2A-GFPm
(SEQ ID NO: 47)
pAC3-E2A-GFP
(SEQ ID NO: 48)
pAC3-GSG-E2A-GFPm
(SEQ ID NO: 49)
pAC3-F2A-GFPm
(SEQ ID NO: 50)
pAC3-GSG-F2A-GFPm
(SEQ ID NO: 51)
pAC3-T2A-yCD2
(SEQ ID NO: 52)
pAC3-GSG-T2A-yCD2
(SEQ ID NO: 53)
pAC3-P2A-yCD2
(SEQ ID NO: 54)
pAC3-GSG-P2A-yCD2

### Example 30: Secretion of scFv-L1 that Lack a Signal Peptide Sequence can be Achieved by Insertion of a Heterologous Signal Peptide at the N-Terminus

Construction of RRV-scFv-PDL1 plasmid DNAs. Two pairs of two different configurations of single-chain variable fragment (scFv) against PD-L1 were designed. One pair consists of scFv with and without the Fc from human IgG1, designated scFv-PDL1 and scFvFc-PDL1, respectively. Another pair consists of scFv-PDL1 and ScFvFc-PDL1 with HA and Flag epitope incorporated at the C-terminus, designated scFv-HF-PDL1 and scFvFc-HF-PDL1. The coding sequence of each configuration contains the 3′ coding sequence of the viral envelope gene followed by the gT2A peptide sequence and was synthesized with Asc I and Not I restriction sites for subcloning into pAC3-gT2A-yCD2 at the corresponding sites to replace the g2A-yCD2 transgene cassette resulting in pAC3-scFv-PDL1, pAC3-scFvFc-PDL1, pAC3-scFv-HF-PDL1, pAC3-scFvFc-HF-PDL1. For all scFv-PDL1 variants, a signal peptide from human IL-2 was incorporated at the N-terminus to allow secretion of scFv PD-L1.

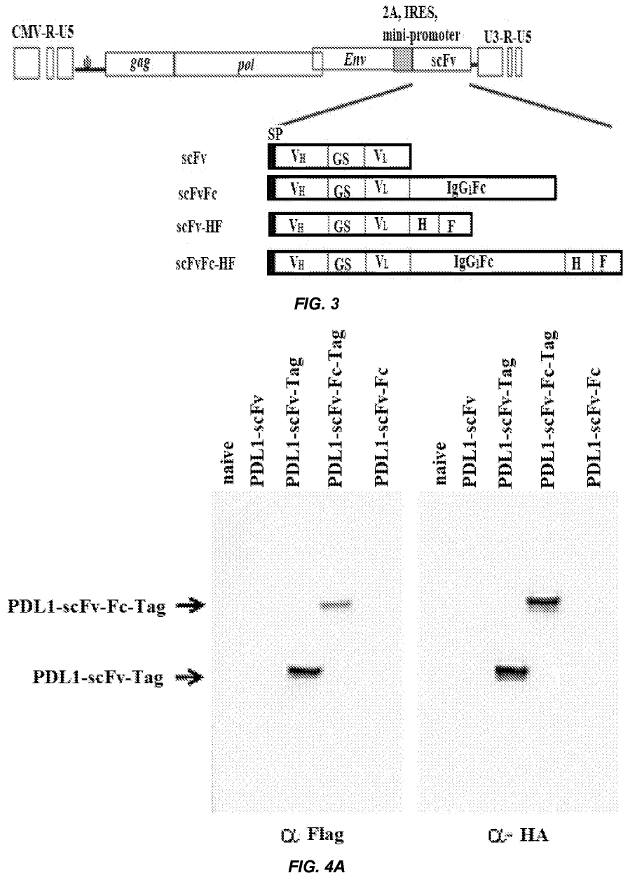

scFv PD-L1 encoded in the RRV-2A configuration is expressed and properly processed. As indicated in FIG. 3 it is possible to express scFv PD-L1 with a heterologous signal peptide by means other than the 2A sequence, such as using an IRES sequence or a minipromoter and obtain a vector that expresses a secretable form of scFV PD-L1. However here we describe the RRV configuration utilizing the viral-derived “self-cleavage” 2A peptide for transgene expression demonstrated that RRV-2A configuration can tolerate transgene insertion up to 1.2 kb. In the current study, we designed two different configurations of a single-chain variable fragment (scFv) against PD-L1. One consists of scFv alone and another with the Fc from human IgG1, designated pAC3-scFv-PDL1 and pAC3-scFvFc-PDL1, respectively. Due to the absence of antibody against scFv PD-L1 protein, we generated a matching pair of the constructs with an HA and Flag epitope incorporated at the C-terminus of the transgene, designated pAC3-scFv-HF-PDL1 and pAC3-scFvFc-HF-PDL1 (FIG. 3).

Transgenes targeted for different cellular compartments encoded in-frame with the viral envelope (Env) protein in the RRV-2A configuration are efficiently separated from Env-transgene polyprotein (Hofacre et al., 2018). Because both the epitope tagged and untagged scFv PD-L1 and scFvFc PD-L1 proteins are designed to be separated from the viral Env protein and secreted from the cells, we used transient transfection system to highly overexpress the transgene proteins to aid the detection of epitope tagged scFv PD-L1 and scFvFc proteins. Cell lysates from transiently transfected 293T cells were resolved on SDS-PAGE and detected with anti-HA and anti-Flag antibody to confirm the presence of scFv PD-L1 and its separation efficiency mediated by the 2A peptide, respectively. In addition, an anti-2A antibody was also included to confirm the proper processing of the viral Env protein from the polyprotein. FIG. 5 shows that both scFv-HF PD-L1 and scFvFc-HF PD-L1 are detected and separated from the polyprotein as expected, and that the viral Env protein is properly processed to its subunits as indicated by the detection of 15E-2A. The residual unseparated polyprotein detected is also expected as the cell lysates are from transiently transfected system in which the protein is highly overexpressed, and it was previously shown that such unseparated polyprotein is not incorporated into viral particles. Further, the detection of intracellular epitope tagged scFv PD-L1 by Western suggests that the protein may not have reached maximal secretion.

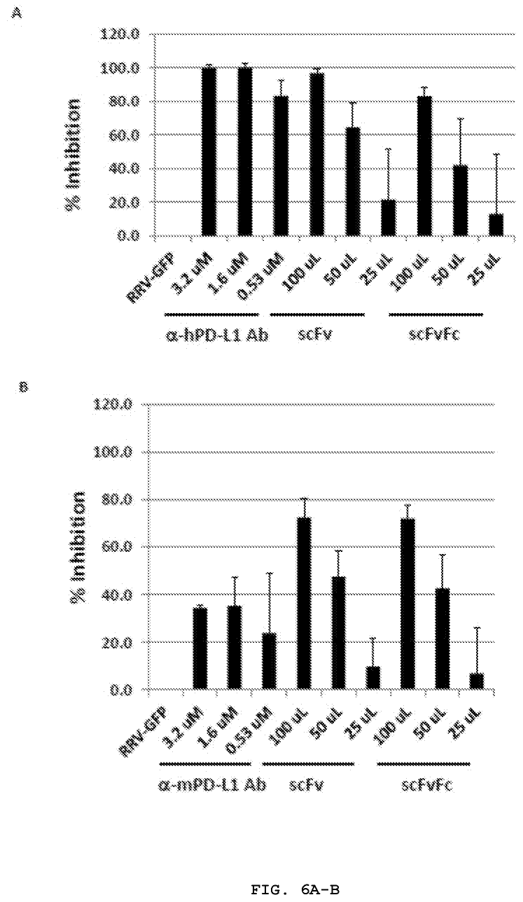

scFv PD-L1 and scFvFc PD-L1 secreted from RRV-scFv-PDL1 and RRV-scFvFc-PDL1 infected cells competes with PD-1 for PD-L1 binding. Having confirmed the transgene protein expression and viral function of RRV-scFv-PDL1 and RRV-scFvFc-PDL1, we evaluated the binding characteristics of scFv PD-L1 and scFvFc PD-L1. The potency of scFv PD-L1 and scFvFc PD-L1 protein to block PD-1/PD-L1 interaction was evaluated using an ELISA-based competition assay to quantify the amount of His-tagged PD-1 that remained bound to PD-L1 after co-incubation of PD-1 with scFv PD-L1 or scFvFc PD-L1. Although the concentration of the scFv PD-L1 and scFvFc PD-L1 in the supernatant is undefined, they specifically bound to human PD-L1 and mouse PD-L1 in a dose-dependent manner. The level of inhibition using 100 μL of the supernatant was comparable to that of the blocking antibody control with no significant difference between scFv PD-L1 and scFvFc PD-L1 (FIG. 6A). The potency of scFv PD-L1 and scFvFc PD-L1 in blocking mouse PD-1/PD-L1 interaction appears to be effective though slightly less potent than with the human counterpart but more effective than the anti-mouse PD-L1 antibody control (FIG. 6B). We further evaluated the binding kinetics of scFv PD-L1 to human and mouse PD-L1 using the surface plasmon resonance system. The scFv PD-L1 cDNA was cloned into a CMV-driven expression vector for transient transfection followed by purification to obtain >85% purity. The equilibrium dissociation constant (K_{D}) of scFv PD-L1 for recombinant human PD-L1 and mouse PD-L1 were determined to be 0.426 nM and 4.78 nM, respectively, Table J. The approximately 10-fold higher binding affinity to human PD-L1 as a result of slower K, could explain the higher potency of scFv PD-L1 in blocking human PD-1/PD-L1 interaction observed in the competitive ELISA, despite the fact that the human and mouse PD-L1 share nearly 80% homology in their amino acid sequences.

TABLE J

| | Temp | kₒₙ × 10⁵ | k_{off} × 10⁻⁴ | K_{D} | T_{1/2} |
|---|---|---|---|---|---|
| Antigen | ° C. | M⁻¹ s⁻¹ | s⁻¹ | nM | Minutes |
| H_PDL1 | 25 | 3.58 ± 0.811 | 1.51 ± 0.352 | 0.426 ± 0.065 | 77 |
| M_PDL1 | 25 | 2.93 ± 0.343 | 13.9 ± 0.45 | 4.78 ± 0.065 | 8.3 |
| H_PDL1 | 37 | 5.28 | 6.27 | 1.21 | 18.4 |
| M_PDL1 | 37 | 5.18 | 65.3 | 12.6 | 1.8 |

### Example 31: scFv PD-L1 Secreted from RRV-scFv-PDL1 Infected Cells Exhibits Bystander Trans-Binding Activity to PD-L1 on the Cell Surface

As infection of 100% of patient tumor cells in situ is not currently feasible by any viral-based therapeutic approach including RRV, we designed a secreted transgene product with the capacity to bind PD-L1 on neighboring, uninfected cells. Here, we employed a cell-based assay to confirm antigen-specific binding of scFv PD-L1 or scFvFc PD-L1 by flow cytometry. In this experiment, due to the lack of antibody to detect the presence of bound scFv PD-L1 and scFvFc PD-L1 on the cell surface, we used the epitope tagged scFv PD-L1 and scFvFc PD-L1 (scFv-HF PD-L1 and scFvFc-HF PD-L1) followed by anti-HA antibody for detection. These data show that scFv-HF PD-L1 and scFvFc-HF PD-L1 bind to PD-L1 expressed on cell surfaces in human and mouse cell lines as indicated by a marked shift in mean fluorescent intensity (MFI) with an anti-HA antibody. A higher shift in MFI observed with scFvFc-HF PD-L1 in both the human and mouse cell lines tested is likely due to bivalent dimer of scFvFc-HF PD-L1 by the dimer formation through the disulfide bond formation between the Fc region, and hence simply a reflection of more anti-HA antibody bound to scFvFc-HF PD-L1 on the cell surface, rather than increased binding affinity, as the scFvFc PD-L1 did not compete more effectively than scFv PD-L1 in the ELISA (FIG. 5). Furthermore, the antigen binding specificity was demonstrated by blocking the accessibility of an anti-PD-L1 blocking antibody to PD-L1 on cell surface when co-incubated with the anti-HA antibody, resulting in a marked decrease in the MFI with the anti-PD-L1 antibody. Consistent with the data observed in the competitive ELISA, scFv-HF PD-L1 and scFvFc-HF PD-L1 bind specifically to PD-L1 on the cell surface and block anti-PD-L1 antibody binding to PD-L1 suggesting the epitope for scFv-HF PD-L1 and scFvFc-HF PD-L1 overlaps or is in proximity to that of the anti-PD-L1 antibody. In addition, the marked decrease in the MFI with anti-PD-L1 antibody also suggests full receptor (PD-L1) occupancy on the cell surface.

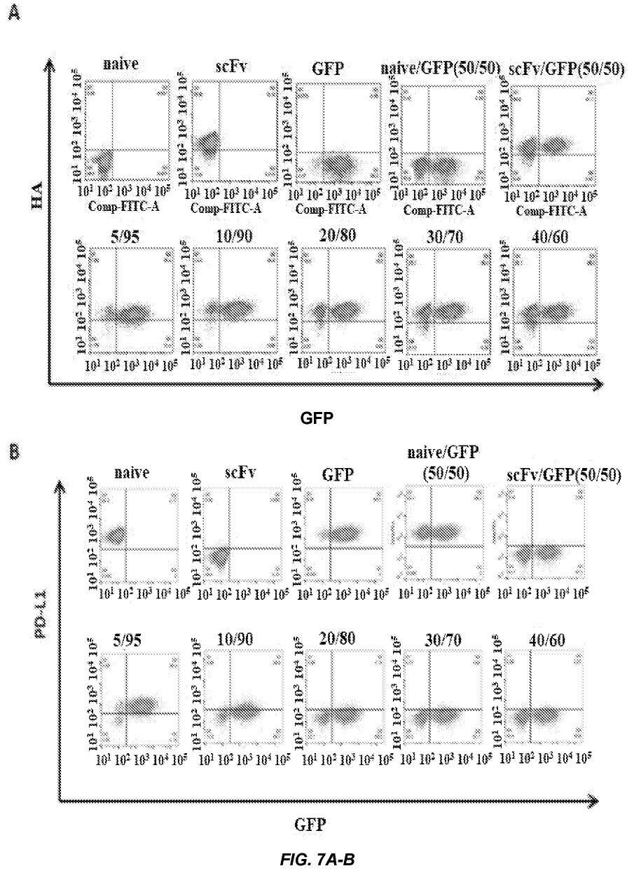

To evaluate the bystander effect of RRV-scFv-PD-L1 in vitro, we tested the minimal transduction level required to achieve full receptor occupancy on tumor cells. In this experiment, EMT6 mouse breast cancer cells maximally infected with RRV-scFv-HF-PD-L1, mixed with EMT6 cells maximally infected with RRV-GFP at various ratios were co-cultured to measure bound scFv-HF PD-L1 and unbound PD-L1 on the cell surface using the anti-HA and anti-PD-L1 antibody. Our data show that bound scFv-HF PD-L1 was detected on all cell surfaces when only 5% of the cells express scFv-HF PD-L1 FIG. 7A. The full occupancy of PD-L1 inversely correlates with the decrease in PD-L1 signal on the cell surface in a dose dependent manner (FIG. 7B), suggesting that scFv PD-L1 can achieve 100% bystander effect with a minimal level of transduction.

### Example 32: scFv PD-L1 and scFvFc PD-L1 Treatment Lead to Tumor Growth Inhibition in a Dose Dependent Manner and Elicit Immune Memory Response in Syngeneic Tumor Models

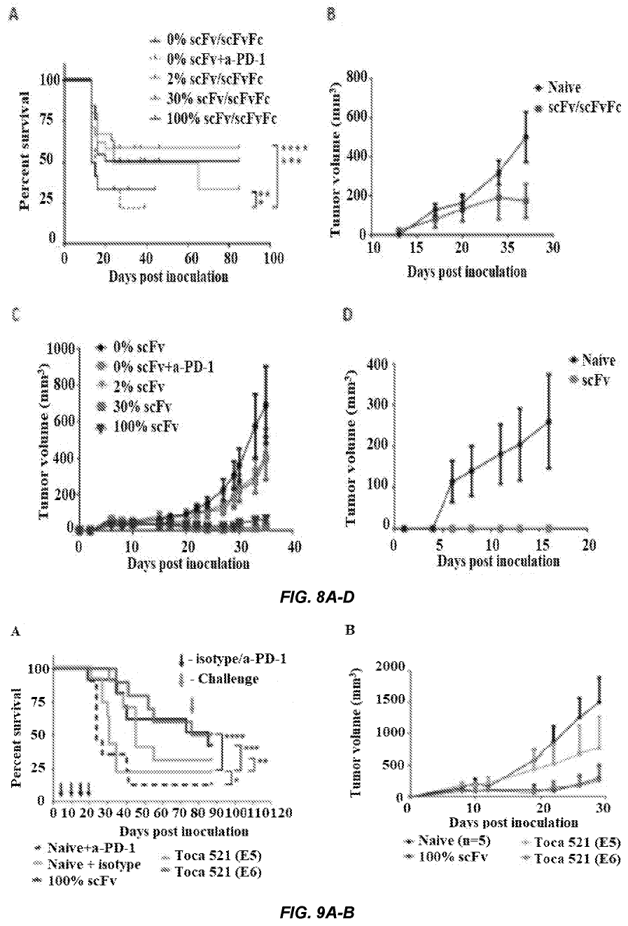

We have shown that in vitro scFv PD-L1 secreted from as low as 5% pre-transduced cells exhibited bystander trans-binding activity, leading to a full PD-L1 occupancy on the cell surface of non-scFv PD-L1 expressing cells. We next evaluated dose response of the anti-tumor activity of scFv PD-L1 in a syngeneic orthotopic EMT6 breast cancer model which has been reported to be responsive to checkpoint inhibitors. To evaluate the anti-tumor activity of scFv PD-L1 and scFvFc PD-L1 in a more clinically relevant scenario, we sought to determine the minimal transduction level required for scFv PD-L1 to achieve anti-tumor activity, using different ratios of EMT6 cells maximally pre-transduced with RRV-scFv-PDL1, RRV-scFvFc-PDL1 or RRV-GFP vectors. These cells are resistant to further RRV infection mediated via the amphotropic envelope protein due to receptor down regulation. In this experiment, mixtures of EMT6 tumor cells pre-transduced with RRV-scFv-PDL1 or RRV-GFP at indicated ratios were implanted in the mammary fat pad in BALB/c mice. Survival was monitored for 90 days and Kaplan-Meier survival analysis was performed to evaluate the anti-tumor activity of scFv PD-L1. As per animal use protocol, mice bearing necrotic tumors were euthanized and censored from analysis (indicated as ticks in FIG. 6A; these mice were not scored as death and were not excluded from the graph). Mice bearing tumors expressing the same ratios of scFv PD-L1 or scFvFc PD-L1 were grouped together for survival analysis. These data show that mice bearing tumors with 2%, 30% and 100% scFv PD-L1 or scFvFc PD-L1 expressing tumor cells trend toward a survival benefit compared to untreated animals, albeit not statistically significant (FIG. 8A) (p=0.2529 for 0% scFv/scFvFc vs anti-PD-1; p=0.2529 for 0% vs 2%; p=0.0919 for 0% vs 30%; p=0.1674 for 0% vs 100%). We further sought to investigate whether mice survived from the primary tumor have established an anti-tumor immune memory response by re-challenging them with naïve EMT6 tumor cells on the flank. FIG. 8B shows that mice that cleared tumor with scFv/scFvFc treatment in the primary setting exhibited a moderate delayed tumor growth in a re-challenge setting suggesting that an anti-tumor immune response was established in these mice. Together the data indicate that tumor cells expressing scFv PD-L1 or scFvFc PD-L1 can lead to anti-tumor activity that appears to be superior to treatment with a commercial antibody.

A Tu-2449SC tumor model was tested in B6C3F1 mice to determine the minimal transduction level required for scFv PD-L1 to exert anti-tumor activity. FIG. 8C shows that in the Tu-2449SC tumor model, mice bearing tumor with as low as 2% Tu-2449SC cells expressing scFv PD-L1 led to a delay in tumor progression that is comparable to anti-PD-1 antibody treatment, and shows a strong trend towards an advantage when compared to control mice (FIG. 8C). With 30% pre-transduced cells, tumor progression was completely inhibited as also seen in mice bearing tumors with the 100% pre-transduced cells.

### Example 33: Intracranial Injection of RRV-scFv-PDL1 Prolongs Survival in Syngeneic Orthotopic Glioma Model

scFv PD-L1 anti-tumor activity was investigated in an orthotopic syngeneic glioma model previously reported to respond to Toca 511 and Toca FC treatment. An intra-tumoral RRV delivery approach previously established (Ostertag et al., 2012) was employed. RRV-scFv-PDL1 viral functions and genome stability in maximally infected Tu-2449 cells were confirmed in vitro. In this experiment, two different doses of RRV-scFv-PDL1 (1E5 and 1E6 TU) were delivered by a single intra-tumoral injection 4 days after tumor implant. The data show that a single administration of 1E6 TU of RRV-scFv-PDL1 is equally effective as Tu-2449 cells maximally pre-transduced with RRV-scFv-PDL1, which were included as a control and as a comparator (FIG. 9A). Consistent with observation made in the previous experiments, subcutaneous re-challenge of Tu-2449SC tumor cells at a remote site from the primary tumor showed a systemic anti-tumor immune response leading to significant delay in tumor growth compared to naïve mice (FIG. 9B). Together, these findings indicate that scFv PD-L1 has anti-tumor activity in a glioma tumor model and represents a second glioma mouse model that responds to checkpoint inhibitors as a monotherapy.

### Example 34: Replacement of the IL-2 Signal Peptide in scFvPD-L1 Encoded in RRV-scFv-PDL1 with the Signal Peptide from Cystatin S and an Artificial Signal Peptide AP1 Increases scFv PD-L1 Protein Secretion In Vitro and Enhances Bystander Effect and Tumor Activity in Multiple Murine Tumor Models

In order to further increase the bystander effect of scFv PD-L1 which may lead to enhanced anti-tumor efficacy, the IL-2 signal peptide was replaced with the one from cystatin S and with an artificial signal peptide (ASP1 from Table B) which is predicted to have high level of secretion. The in vitro bystander experiment reveals that infected cells expressing the epitope tagged scFv PD-L1 carrying the signal peptide from cystatin S (RRV-CSscFv-PDL1) and ASP1 (RRV-AP1scFv-PDL1) exhibit a higher trans-binding activity to PD-L1 on neighboring bystander cells. Whereas 5-10% of RRV-scFv-PDL1 cells were required to saturate all the cell surface PD-L1 on the bystander cells, only 2-4% of RRV-CSscFv-PDL1 infected or RRV-AP1scFv-PDL1 infected cells is required to reach full PD-L1 receptor occupancy on the bystander cells.

A Tu2449SC tumor model with 2% pre-transduced tumor is used to compare the anti-tumor activity among tumors infected with RRV-scFv-PD-L1, RRV-CSscFv-PD-L1 and RRV-AP1scFv-PD-L1. As the 2% transduction level has previously shown to be less efficacious than 30% pre-transduced tumor infected with RRV-scFv-PD-L1, we expect the greater bystander effect observed with RRV-CSscFv-PD-L1 and RRV-AP1scFv-PD-L1 in vitro will show greater anti-tumor activity in the 2% pre-transduced setting. Our data reveal that the anti-tumor effect of scFv PD-L1 produced from RRV-CSscFv-PD-L1 and RRV-AP1scFv-PD-L1 infected tumor is significantly higher than scFv PD-L1 produced from RRV-scFv-PDL1. Our data support the notation that choices of signal peptide can also modulate the level of protein secretion leading to enhanced anti-tumor activity.

### Example 35: Incorporation of a Potent Signal Peptide at the N-Terminus of an Antigen-Specific Binder (ASB) Derived from Scaffold Protein can Also be Expressed by RRV

Construction of RRV-ASB-PDL1 plasmid DNAs. One pair of same configurations of ASB against PD-L1 are designed. One consists of ASB and another with HA and Flag epitope incorporated at the C-terminus, designated ASB-HF-PDL1 and ASB-HF-PDL1. The coding sequence of each configuration contains the 3′ coding sequence of the viral envelope gene followed by the gT2A peptide sequence and is synthesized with Asc I and Not I restriction sites for subcloning into pAC3-gT2A-yCD2 at the corresponding sites to replace the g2A-yCD2 transgene cassette resulting in pAC3-ASB-PDL1 and pAC3-ASB-HF-PDL1. For all ASB-PDL1 variants, a signal peptide from human IL-2 is incorporated at the N-terminus to allow secretion of ASB PD-L1 or ASB-HF PD-L1.

In vitro bystander experiment shows that infected cells expressing the epitope tagged ASB PD-L1 exhibit trans-binding activity to PD-L1 on neighboring bystander cells comparable to scFv PD-L1, where 5% RRV-scFv-PDL1 infected cells or 5% RRV-ABS-PdL1 infected cells are required to saturate all the cell surface PD-L1 on the bystander cells.

Subsequently, dose response of the anti-tumor activity of ASB PD-L1 is evaluated in parallel to scFv PD-L1 in a syngeneic Tu2449SC subcutaneous model. The in vivo data show that ASB PD-L1 has anti-tumor activity. Mice bearing tumor with as low as 2% Tu-2449SC cells expressing ASB PD-L1 lead to a delay in tumor progression that is comparable to 2% Tu-2449SC cells expressing scFv PD-L1 or anti-PD-1 antibody treatment, but not statistically significant when compare to control mice. With 30% pre-transduced cells, tumor progression is completely inhibited as also seen in mice bearing tumors with the 100% pre-transduced cells.

### Example 36: Intracranial Injection of RRV-scFv-PDL1-yCD2 Prolongs Survival in Syngeneic Orthotopic Glioma Model

scFv PD-L1 anti-tumor activity is investigated in combination with yCD2 and 5-FC to evaluate their synergistic effect in an orthotopic syngeneic glioma model. A dual vector is designed with a cassette consists of the the human IL-2 signal peptide, scFv-PDL1 linked to gP2A-yCD2. The fragment is synthesized and cloned into RRV-gT2A backbone at the AscI and NotI sites. The resulting vector is designated pAC3-scFv-PDL1-yCD2. In vitro characterization data show that scFv PDL1 and yCD2 proteins are expressed from RRV-scFv-PDL1-yCD2 infected cells and retain their biological functions (i.e. scFv PD-L1 binds to PD-L1 and yCD2 converts 5-FC to 5-FU). Purified RRV-scFv-PDL1 and RRV-scFv-PDL1-yCD2 vectors are produced for in vivo studies. In this experiment, dose of 1E5 TU of RRV-scFv-PDL1 which shows suboptimal anti-tumor activity as a monotherapy (FIG. 9A), and 1E5 TU of RRV-scFv-PDL1-yCD2 are delivered by a single intra-tumoral injection 4 days after tumor implant with. Following 10 days to allow viral spread and anti-tumor activity of scFv PD-L1, miced are then treated IP once daily for 7 day on and 7 day off with either PBS or 5-FC (500 mg/kg). Our data show that a single administration of 1E5 TU of RRV-scFv-PDL-yCD2 treated with 5-FC is superior to RRV-scFv-PDL1 and RRV-scFv-PDL-yCD2 treated with PBS. Consistent with observation made in the previous experiments, subcutaneous re-challenge of Tu-2449SC tumor cells at a remote site from the primary tumor shows a systemic anti-tumor immune response leading to significant delay in tumor growth compared to naïve mice. Some rechallenged mice are tumor free for up to 90 days. These data indicate that combination therapy of scFv PD-L1 and yCD2/5FC has superior anti-tumor activity than scFv PD-L1 monotherapy in a glioma tumor model.

### Example 37: RRV-g T2A-Affimer-SQT Produced from 293T Cells is Infectious and Expresses a Secretable Form of the Affimer-SQT Protein

The coding region of the SQT variant of Affimer was obtained from Stadler et al. (Protein Engineering, Design and Selection, 24(9) 751-763, 2011). For detection of Affimer-SQT protein expression, HA, AU1 and Myc etitope were inserted at the N-terminus (preceeding the signal peptide), L1 and L2 of the Affimer-SQT, respectively. A signal peptide derived from human IL-2 was placed at the N-terminus of the Affimer-SQT coding region. The DNA fragment was synthesized and cloned into AscI and Not I sites in the RRV gT2A backbone. The resulting construct is designated pAC3-gT2A-Affimer-SQT.

HEK293T cells were seeded at 2e6 cells per 10 cm plates the day before transfection. The next day, calcium phosphate transfection was performed using 20 μg of plasmid DNA. Eighteen hours post-transfection, cells were washed with DMEM twice and replaced with complete culture medium. Viral supernatant was collected approximately 24 hours post medium replacement and filtered through a 0.45 μm syringe filter. The viral titer of RRV-g T2A-Affimer-SQT was determined as described previously (Perez et al., 2012). Table K shows that titer of RRV-g T2A-Affimer-SQT produced from HEK293T cells were comparable to that of RRV-GFP.

The Affimer-SQT protein encoded in pAC3-gT2A-Affimer-SQT is designed to be secreted into the supernatant. Due to the uncertainty of the Affimer-SQT protein amount present in the supernatant, detection Affimer-SQT protein in the supernatant was performed by both direct immunblotting of 15 μL of the supernatant using an anti-HA antibody (Sigma Cat #H6908, 1:1000) or immunoprecipitation by incubating 1 mL of the supernatant with 10 μg anti-myc antibody (Abcam Cat #ab206486) for 16 18 hours at 4° C. followed by immunoblotting with an anti-HA antibody and a HPR-conjugated secondary antibody. FIG. 10 shows that Affimer SQT is expressed abundantly in the supernatant with expected molecular weight of ˜15 kDa.

TABLE K
Titer of RRV-gT2A-Affimer-SQT produced from
transiently transfected 293T cells.

| | TU/mL |
|---|---|
| RRV-GFP | 3.36E+6 |
| RRV-gT2A-Affimer-SQT | 3.70E+6 |

### Example 38: RRV-gT2A-Hck and RRV-IRES-Hck Produced from 293T Cells is Infectious and Expresses the Hck Protein

The coding region of the Hck was obtained from Patent WO2017009533A1. For detection of Hck protein expression, Flag and His epitope tags were inserted at the C-terminus of Hck, and a signal peptide derived from human IL-2 was placed at the N-terminus of the Hck coding region. The DNA fragment with AscI and Not I sites was synthesized and cloned into AscI and Not I sites in the RRV-gT2A backbone and the DNA fragment with PsiI and Not I sites was synthesized and cloned into PsiI and Not I sites in the RRV-IRES backbone resulting constructs designated pAC3-gT2A-Hck and pAC3-IRES-Hck, respectively.

RRV viral supernatant and Hck protein were produced in HEK293T cells as described. Table L shows that titer of RRV-gT2A-Hck produced from HEK293T cells were comparable to that of RRV-GFP.

TABLE L
Titer of RRV-gT2A-Hck and RRV-IRES-Hck produced
from transiently transfected 293T cells.

| | TU/mL |
|---|---|
| RRV-GFP | 3.36E+6 |
| RRV-gT2A-Hck | 6.07E+6 |
| RRV-IRES-Hck | 2.00E+6 |

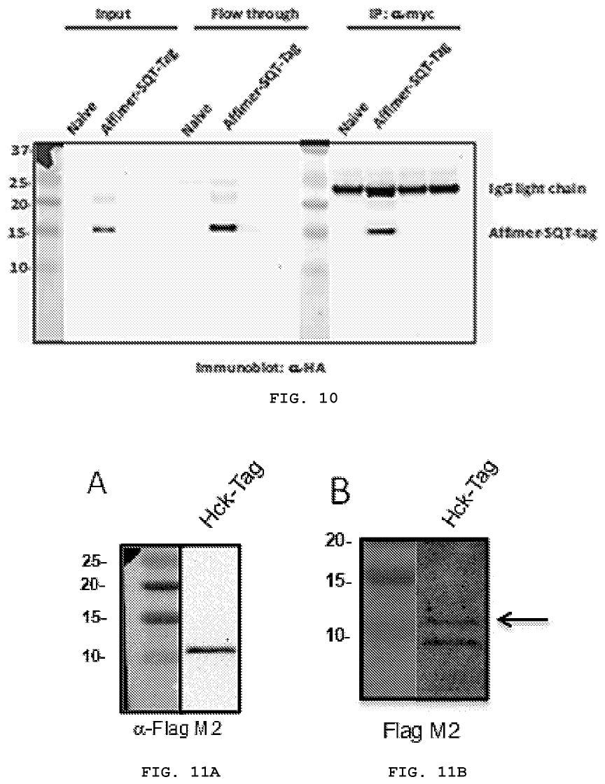

The Hck protein encoded in pAC3-gT2A-Hck is designed to be secreted into the supernatant. Detection the Hck protein in the supernatant was performed by direct immunoblotting of 15 μL of the supernatant using an anti-Flag M2 antibody (Sigma Cat #F1804, 1:1000) and a HPR-conjugated secondary antibody. FIG. 11 shows that Hck protein is expressed abundantly in the supernatant with expected molecular weight of ˜7 kDa.

### Example 39: RRV-gT2A-Anticalin Produced from 293T Cells is Infectious and Expresses the Anticalin Protein

The coding region of the Anticalin-Lcn2 is obtained from Gebauer et al., 2013 (JMB 425(4) 780-802). For detection of the Anticalin-Lcn2 protein expression, Flag and His epitope tags are inserted at the C-terminus of Anticalin-Lcn2, and a signal peptide derived from human IL-2 is placed at the N-terminus of the Anticalin-Lcn2 coding region. The DNA fragment is synthesized and cloned into AscI and Not I sites in the RRV-gT2A backbone. The resulting construct is designated pAC3-gT2A-Anticalin-Lcn2.

The Anticalin-Lcn2 protein encoded in pAC3-gT2A-Anticalin-Lcn2 is designed to be secreted into the supernatant. Detection the Anticalin-Lcn2 protein in the supernatant is performed by direct immunoblotting of 15 μL of the supernatant using an anti-Flag M2 antibody (Sigma Cat #F1804, 1:1000) and a HPR-conjugated secondary antibody. The data shows that Anticalin-Lcn2 protein is expressed abundantly in the supernatant with expected molecular weight of ˜20 kDa.

### Example 40: Backbone Framework Amino Acid Residues and Surface-Exposed Amino Acid Residues Involved in Antigen-Binding as Well as Amino Acids Residues in the Oligomerization Domains can be Optimized to Become Apobec-Resistant

One important aspect of scaffold proteins is to maintain the overall integrity or the structure of the scaffold. To avoid Apobec3-mediated mutation which could result in coding a non-sense/STOP codon (nucleic acid TGA TAA and TAG) during viral infection, introducing nucleic acid substitutions that renders the therapeutic transgene coding sequence Apobec3-resistant is employed by substituting selective or all tryptophan residues present in the scaffold backbone framework and/or surface-exposed amino acids involved in antigen binding with other 19 amino acids to avoid a non-sense/STOP codon hypermutation mediated by Apobec3.

Anticalin derived from Lcn2 (Gebauer et al., 2012 J Mol Biol 425(4):780-802) contains two tryptophan residues: one presents in the beta-strand A and another in the beta-strand D. In addition, an ED-B binder Anticalin, N7A, contains 3 additional tryptophan residues in the beta-strand D and Loop 3/beta-strand F. Computation algorithms (Parthiban et al., BMC Sturctural Biology 2007 7:54; Bywate, PLoS 2016 11(3):e150769) are employed and a combinatorial mutagenesis library of the 19 amino acids for the selected tryptophan residues YAfiez et al. (Nucleic Acids Reseasrch 32(20)e158, 2004) is generated to evaluate and test for their expression, antigen-binding affinity. Our data show that tryptophan residues involved in structural integrity present in the backbone framework and in the antigen-binding loops of Anticalin N7A can be replaced by conservative amino acid residues such as tyrosine and phenylalanine. The Apobec-resistant N7A variants when encoded in RRV-gT2A backbone show comparable protein expression level with that of the parental N7A protein. Most importantly, the purified Apobec-resistant N7A protein expressed from pcDNA3.1 vector in 293F cells shows comparable secondary structure when analyzed by far-UV circular dichroism spectroscopy and similar binding affinity to EB-D by SPR-based biosensor analysis.

The tolerability of replacing tryptophan with tyrosine or phenylalanine in a scaffold framework is also demonstrated in the Hck protein in which two consecutive tryptophan residues present adjacent to the src-loop can be replaced with two phenylalanines (FF), two tyrosine (YY), tyrosine-phenylalanine (YF) or phenylalanine-tyrosine (FY) without compromising its expression. In addition, we also show that the tryptophan residue in the Type I deiodinase dimerization motif can be substituted with phenylalnine and tyrosine without compromising its dimerization function.

### Example 41: Epitope Tagged Affimer-SQT can be Expressed in a Homodimeric Form in RRV-gT2A Backbone Using an Fc Region of Human IgG

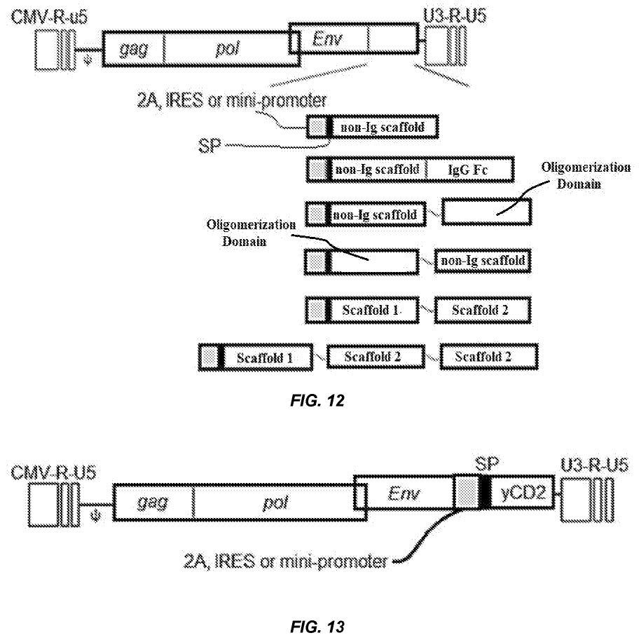

To express a homodimer of the Affimer-SQT, in addition to the incorporation of the human IL-2 signal peptide at the N-terminus, the coding sequence of the Affimer-SQT is linked with a (G4S)3 glycine-serine linker followed by IgG4 Fc region. The design of vectors encoding this type of non-IG binding protein is shown in FIG. 12, along with other types of modifications that compress genes encoding binding proteins that allow the formation of multimers, or multiple binding specificities to form a bispecic antibody or antibody-like bi- or tri specific molecules. The synthesized fragments are cloned into AscI and Not I sites in the RRV gT2A backbone. One resulting construct is designated pAC3-gT2A-Affimer-SQT-Fc. Data shows that under non-reducing condition, a dimeric form Affimer-SQT is detected using an anti-human IgG4 Fc antibody with an expected molecular size of ˜50 kDa.

### Example 42: Epitope Tagged Affimer-SQT can be Expressed in a Homodimeric Form Using a Dimerization Domain

To express a homodimer of the Affimer-SQT, in addition to the incorporation of the human IL-2 signal peptide at the N-terminus of the Affimer-SQT, and epitope tags at the C-terminus of the Affimer-SQT, the dimerization domain (Table 6) of Type I deiodinase linked with a GGGG glycine-linker on both N- and C-terminus is placed downstream of the signal peptide followed by the Affimre-SQT. In another configuration, the human IL-2 signal peptide and the epitope tags are placed at the N-terminus of the Affimer-SQT and the dimerization domain linked with a GGGG glycine-linker on both N- and C-terminus is placed at the C-terminus of the Affimer-SQT. The synthesized fragments are cloned into AscI and Not I sites in the RRV gT2A backbone. The resulting constructs are designated pAC3-gT2A-2Affimer-SQT and pAC3-gT2A-Affimer-SQT2, respectively.

Protein expression data show that under non-reducing condition, more than 85% of 2Affimer SQT and Affimer SQT2 protein are detected in a dimeric form with an expected molecular size of ˜32 kDa.

### Example 43: Epitope Tagged Affimer-SQT can be Expressed in a Homotrimeric Form Using a Trimerization Domain

To express a homotrimer of Affimer-SQT, in addition to the incorporation of the human IL-2 signal peptide at the N-terminus of the Affimer-SQT, and epitope tags at the C-terminus of the Affimer-SQT, the trimerization domain (Table 6) of Coronin 1a with a GGGG glycine-linker on both N- and C-terminus is placed downstream of the signal peptide followed by the Affimre-SQT. In another configuration, the human IL-2 signal peptide and the epitope tags are placed at the N-terminus of the Affimer-SQT and the trimerization domain linked with a GGGG glycine-linker on both N- and C-terminus is placed at the C-terminus of the Affimer-SQT. The synthesized fragments are cloned into AscI and Not I sites in the RRV gT2A backbone. The resulting constructs are designated pAC3-gT2A-3Affimer-SQT and pAC3-gT2A-Affimer-SQT3, respectively.

Protein expression data show that under non-reducing condition, more than 85% of 3Affimer SQT and Affimer SQT3 protein are detected in a trimeric form with an expected molecular size of ˜56-kDa.

### Example 44: Epitope Tagged Affimer-SQT can be Expressed in a Homotetrameric Form Using a Tetrameric Domain

To express a homotetramer form of Affimer-SQT, in addition to the incorporation of the human IL-2 signal peptide at the N-terminus of the Affimer-SQT, and epitope tags at the C-terminus of the Affimer-SQT, cartilage matrix protein (CMP) CMP(R27Q) tetrameric domain (Table 6) linked with a GGGG glycine-linker on both N- and C-terminus is placed downstream of the signal peptide followed by the Affimre-SQT. In another configuration, the human IL-2 signal peptide and the epitope tags are placed at the N-terminus of the Affimer-SQT and the tetramerization domain linked with a GGGG glycine-linker on both N- and C-terminus is placed at the C-terminus of the Affimer-SQT. The synthesized fragments are cloned into AscI and Not I sites in the RRV gT2A backbone. The resulting constructs are designated pAC3-gT2A-4Affimer-SQT and pAC3-gT2A-Affimer-SQT4, respectively.

Protein expression data show that under non-reducing condition, more than 85% of 4Affimer SQT and Affimer SQT4 protein are detected in a tetrameric form with an expected molecular size of ˜56 kDa.

### Example 45: Epitope Tagged Affimer-SQT can be Expressed in Homopentameric Form Using a Pentamerization Domain

To express a homopentamer of Affimer-SQT, in addition to the incorporation of the human IL-2 signal peptide at the N-terminus of the Affimer-SQT, and epitope tags at the C-terminus of the Affimer-SQT, the cartilage oligomeric matrix protein (COM P) pentameric domain (Table 6) linked with a GGGG glycine-linker on both N- and C-terminus is placed downstream of the signal peptide followed by the Affimre-SQT. In another configuration, the human IL-2 signal peptide and the epitope tags are placed at the N-terminus of the Affimer-SQT and the pentamerization domain linked with a GGGG glycine-linker on both N- and C-terminus is placed at the C-terminus of the Affimer-SQT. The synthesized fragments are cloned into AscI and Not I sites in the RRV gT2A backbone. The resulting constructs are designated pAC3-gT2A-5Affimer-SQT and pAC3-gT2A-Affimer-SQT5, respectively.

Protein expression data show that under non-reducing condition, more than 85% of 5Affimer SQT and Affimer SQT5 proteins are detected in a tetrameric form with an expected molecular size of ˜100 kDa.

### Example 46: Epitope Tagged Affimer-SQT can be Expressed in Homohexameric Form Using the Hexamerization Domain Derived from IgM

To express a homohexamer of Affimer-SQT, in addition to the incorporation of the human IL-2 signal peptide at the N-terminus of the Affimer-SQT, and epitope tags at the C-terminus of the Affimer-SQT, the human IgM Cμ4tp hexamerization domain (Table 4) linked with a GGGG glycine-linker on both N- and C-terminus is placed downstream of the signal peptide followed by the Affimre-SQT. In another configuration, the human IL-2 signal peptide and the epitope tags are placed at the N-terminus of the Affimer-SQT and the hexamerization domain linked with a GGGG glycine-linker on both N- and C-terminus is placed at the C-terminus of the Affimer-SQT. The synthesized fragments are cloned into AscI and Not I sites in the RRV gT2A backbone. The resulting constructs are designated pAC3-gT2A-6Affimer-SQT and pAC3-gT2A-Affimer-SQT6, respectively.

Protein expression data show that under non-reducing condition, more than 95% of 6Affimer SQT and Affimer SQT6 proteins are detected in a tetrameric form with an expected molecular size of ˜175 kDa.

### Example 47: Epitope Tagged Affimer-SQT and Hck can be Expressed in Hetero-Dimeric Form in RRV gT2A Backbone Using the (G4S)3 Glycine-Serine Linker

To express a heterodimeric of Affimer-SQT and Hck, the coding sequences of the Affimer-SQT and Hck are linked with a (GGGGS)3 glycine-serine linker in two possible configurations (Affimer-SQT-g-Hck and Hck-g-Affimer-SQT) with incorporation of the human IL-2 signal peptide at the N-terminus and epitope tags at the C-terminus of the “fusion” protein. The synthesized fragments are cloned into AscI and Not I sites in the RRV gT2A backbone. The resulting constructs are designated pAC3-gT2A-Affimer-SQT-g-Hck and pAC3-gT2A-Hck-g-Affimer-SQT, respectively.

Protein expression data show that a heterodimeric form of Affimer-SQT-g-Hck and Hck-g-Affimer-SQT are detected with an expected molecular size of ˜23 kDa.

### Example 48: Epitope Tagged Affimer-SQT and Anticalin can be Expressed in Hetero-Dimeric Form in RRV gT2A Backbone Using the (G4S)3 Glycine-Serine Linker

To express a heterodimeric of the Affimer-SQT and Anticalin, the coding sequences of the Affimer-SQT and Anticalin are linked with a (GGGGS)3 glycine-serine linker in two possible configurations (Affimer-SQT-g-Anticalin and Anticalin-g-Affimer-SQT) with incorporation of the human IL-2 signal peptide at the N-terminus and epitope tags at the C-terminus of the “fusion” protein. The synthesized fragments are cloned into AscI and Not I sites in the RRV gT2A backbone. The resulting constructs are designated pAC3-gT2A-Affimer-SQT-g-Anticalin and pAC3-gT2A-Anticalin-g-Affimer-SQT, respectively.

Protein expression data show that a heterodimeric form of Affimer-SQT-g-Anticalin and Anticalin-g-Affimer-SQT are detected with an expected molecular size of ˜36 kDa.

### Example 49: Epitope Tagged Anticalin and Hck can be Expressed in Hetero-Dimeric Form in RRV gT2A Backbone Using the (G4S)3 Glycine-Serine Linker

To express a heterodimeric of the Hck and Anticalin, the coding sequences of the Hck and Anticalin are linked with a (GGGGS)3 glycine-serine linker in two possible configurations (Hck-g-Anticalin and Anticalin-g-Hck) with incorporation of the human IL-2 signal peptide at the N-terminus and epitope tags at the C-terminus of the “fusion” protein. The synthesized fragments are cloned into AscI and Not I sites in the RRV gT2A backbone. The resulting constructs are designated pAC3-gT2A-Hck-g-Anticalin and pAC3-gT2A-Anticalin-g-Hck, respectively.

Protein expression data show that a heterodimeric form of Hck-g-Anticalin and Anticalin-g-Hck are detected with an expected molecular size of ˜28 kDa.

### Example 50: Epitope Tagged Affimer-SQT, Hck and Anticalin can be Expressed in Hetero-Trimeric Form in RRV gT2A Backbone Using the (G4S)3 Glycine-Serine Linker

To express a heterotrimeric of theAFfimer-SQT, Hck and Anticalin, the coding sequences of the Affimer-SQT, Hck and Anticalin are linked with a (GGGGS)3 glycine-serine linker with incorporation of the human IL-2 signal peptide at the N-terminus and epitope tags at the C-terminus of the “fusion” protein. The fragments with six possible combinations (Hck-g-Affimer-SQT-g-Anticalin, Hck-g-Anticalin-g-Affimer-SQT, Affimer-SQT-g-Hck-g-Anticalin, Affimer-SQT-g-Anticalin-g-Hck, Anticalin-g-Hck-g-Affimer-SQT, and Anticalin-g-Affimer-SQT-g-Hck) are synthesized and cloned into AscI and Not I sites in the RRV gT2A backbone. The resulting constructs are designated pAC3-gT2A-Hck-g-Affimer-SQT-g-Anticalin and pAC3-gT2A-Hck-g-Anticalin-g-Affimer-SQT, pAC3-gT2A-Affimer-SQT-g-Hck-g-Anticalin,pAC3-gT2A-Affimer-SQT-g-Anticalin-g-Hck, pAC3-gT2A-Anticalin-g-Hck-g-Affimer-SQT, and pAC3-gT2A-Anticalin-g-Affimer-SQT-g-Hck, respectively.

Protein expression data show that a heterotrimeric form of Hck-g-Affimer-SQT-g-Anticalin, Hck-g-Anticalin-g-Affimer-SQT, Affimer-SQT-g-Hck-g-Anticalin, Affimer-SQT-g-Anticalin-g-Hck, Anticalin-g-Hck-g-Affimer-SQT, and Anticalin-g-Affimer-SQT-g-Hck are detected with an expected molecular size of ˜43 kDa.

### Example 51: RRV-S1-Anticalin Produced from 293T Cells is Infectious and Express the Anticalin Protein Mediated by a Core Promoter

The coding region of the Anticalin-Lcn2 is obtained from Gebauer et al., 2013 (JMB 425(4) 780-802). For detection of the Anticalin-Lcn2 protein expression, Flag and His epitope tags are inserted at the C-terminus of Anticalin-Lcn2, and a signal peptide derived from human IL-2 is placed at the N-terminus of the Anticalin-Lcn2 coding region and downstream of a core promoter. These core promoters are, but not limited to, based on the adenovirus major late (AdML) and cytomegalovirus (CMV) major immediate early genes, and the synthetic “super core promoter” SCP1 (see, also, U.S. Pat. Publ. No. 2015/0273029A1, the disclosure of which is incorporated herein by reference in its entirety). The DNA fragments containing the core promoter AdML-Anticalin-Lcn2, CMV-Anticalin-Lcn2 and SCP1-Anticalin-Lcn2 are synthesized and cloned in the pAC3-derived RRV backbone, resulting constructs designated pAC3-A1-Anticalin-Lcn2, pAC3-C1-Anticalin-Lcn2, and pAC3-S1-Anticalin-Lcn2, respectively.

The Anticalin-Lcn2 protein encoded in pAC3-A1-Anticalin-Lcn2, pAC3-C1-Anticalin-Lcn2, and pAC3-S1-Anticalin-Lcn2 is designed to be secreted into the supernatant. Detection the Anticalin-Lcn2 protein in the supernatant is performed by direct immunoblotting of 15 μL of the supernatant using an anti-Flag M2 antibody (Sigma Cat #F1804, 1:1000) and a HPR-conjugated secondary antibody. Our data shows that Anticalin-Lcn2 protein is expressed abundantly in the supernatant with expected molecular weight of ˜20 kDa.

### Example 52: Tu2449-MG Cells Infected RRV-GSG-T2A-syCD2 (Secreted Modified Yeast Cytioi Duminais) Show Delayed 5 FU Cytotoxicity but Greater Bystander Effect Compared to that of RRV-GSG-T2A-yCD2

pAC3-IRES-syCD2 and pAC3-GSG-T2A-syCD2 are generated to express secreted yCD2 (syCD2). Previously secreted cytosine deamase from bacteria in a non-replicative adenoviral vector has been investigated (Rehemtulla et al. antixcan Res., 23:1393-1400 2004) because it was feard that, with the non-secretd form, the transduced cells were killed by local production of 5-FU before much bystander killing occurred. There are several significant differences between Rehemtulla and the investigation described here. These include: 1) Rehemtulla was investigating bacterial cytosine deaminase (bCD) which has a 20 fold lower affinity for 5-FC compared to wild type yeast cytosine (Kievet et al. Can Res. 59: 1417-1421 1999); the animal model data shows inefficient tumor inhibition in both secreted and cytoplasmic bCD, compared to yeast-derived yCD2 (Rhemtulla et al.; Ostertag et al NeuroOnc 2012); the vector used by rehemtula was non-replicative unlike the RRV encoded yCD2, which spreads from cell to cell. Therefore the effect on cell killing and the bystander effect is more complex and not predictable for yCD from Rehemtulla's bCD data.

The IRES-scyCD2 and GSG-T2A-syCD2 cassettes are designed so that a SSP derived from human IL-2 is placed in-frame at the N-terminus of the yCD2 for pAC3-IRES-syCD2 or between the GSG-T2A and yCD2 for pAC3-GSG-T2A-syCD2. The cassettes are chemically synthesized (Genewiz) with PsiI and Not I sites for pAC3-IRES-syCD2 and AscI and NotI sites for pAC3-GSG-T2A-syCD2 and cloned into pAC3 as pAC3-IRES-syCD2 and pAC3-GSG-T2A-yCD2 backbone, respectively to replace yCD2. The syCD2 protein expression is evaluated from both cell lysates and supernatant collected from transfected HEK293T cells using the anti-yCD2 antibody. In contrast to intracellular expression of yCD2 derived from IRES-yCD2 and GSG-T2A-yCD2, the result demonstrates that inclusion of the human IL 2 SSP in IRES-syCD2 and GSG-T2A-syCD2 results in detection of robust expression of syCD2 in the supernatant and lower or undetectable in cell lysates. Secretion of syCD2 in both constructs is efficient as indicated by a minimal input of 10 μL supernatant in the immunoblotting assay. Furthermore, the extracellular form of syCD2 is similar in size compare to their parental constructs (pAC3-IRES-yCD2 and pAC3-GSG-T2A-yCD2). In addition, viral supernatant of RRV-IRES-syCD2 and RRV-GSG-T2A-syCD2 collected from transiently transfected HEK293T cells show titer values of 0.5-5E6 TU/mL and is comparable to that of RRV-IRES-syCD2 (1.5E6 TU/mL) and RRV-GSG-T2A-yCD2(2E6 TU/mL, respectively.

The extracellular 5-FU concentrations in Tu2449 cells maximally infected with Tu2449/RRV-IRES-syCD2 and Tu2449/RRV-GSG-T2A-syCD2 were compared to Tu24449/RRV-IRES-yCD2 and Tu2449/RRV-GSG-T2A-yCD2, respectively. The data indicate the concentrations of the 5-FU present after 5-FC addition in supernatant from Tu2449/RRV-IRES-syCD2 and Tu2449/RRV-GSG-T2A-syCD2 cells, after 1 hr reaction with excess 5-FC increases over cell growth time in the culture media and reach maximum levels by days 2 to 6 from initial cell seeding. The 5-FU concentrations present in supernatant of Tu2449/RRV-IRES-syCD2 and Tu2449/RRV-GSG-T2A-syCD2 are up to 4-log of a magnitude higher than that of Tu2449/RRV-IRES-yCD2 and Tu2449/RRV-GSG-T2A-yCD2. Subsequently, the effectiveness of 5 EU bystander effect was evaluated in tissue culture by generating matching pairs of RRV-transduced Tu2449 cells infected with RRV-IRES-yCD2/RRV-IRES-GFP and RRV-IRES-syCD2/RRV-IRES-GFP, RRV-GSG-T2A-yCD2/RRV-GSG-T2A-GFP and RRV-GSG-T2A-syCD2/RRV-GSG-T2A-GFP at ratios of 3/97, 15/85, and 30/70 and treating the cultures with 5-FC. In these experiments the GFP vector infected cells are blocked from further infection so no further viral spread of CD encoding vector occurs The in vitro cell killing data at the ratio of 3/97 and 15/85 setting indicate that both RRV-IRES-syCD2 and RRV-GSG-T2A-syCD2 have more bystander-mediated cytotoxic effect than RRV-IRES-yCD2 and RRV-GSG-T2A-yCD2. IRES-syCD2 and RRV-GSG-T2A-syCD2 show more efficient cell killing compared to that with RRV-IRES-yCD2 as well as in RRV-GSG-T2A-yCD2, respectively.

### Example 53: Subcutaneous, Syngeneic Glioma Tumors in Mice Treated with RRV-GSG-T2A-syCD2 or RRV-IRES-syCD2 Showed Delayed Tumor Growth Comparable to that of RRV-GSG-T2A-yCD2 or RRV-GSG-T2A-yCD2, Respectively

To test if secretion of syCD2 from infected tumor cells results in an improved antitumor response in vivo, Tu2449 cells are used to establish a syngeneic orthotopic glioma model in B6C3F1 mice. As described previously, matching pairs of RRV-transduced Tu2449 cells infected with RRV-IRES-yCD2/RRV-IRES-GFP and RRV-IRES-syCD2/RRV-IRES-GFP, RRV-GSG-T2A-yCD2/RRV-GSG-T2A-GFP and RRV-GSG-T2A-syCD2/RRV-GSG-T2A-GFP at ratios of 3/97, 15/85, and 30/70 are generated. A dose-dependent survival benefit compared to animals without 5-FC treatment, is observed within each subgroup of RRV-IRES-yCD2/RRV-IRES-GFP, RRV-IRES-syCD2/RRV-IRES-GFP, RRV-GSG-T2A-yCD2/RRV-GSG-T2A-GFP and RRV-GSG-T2A-syCD2/RRV-GSG-T2A-GFP. However, when the survival data over a 90-day period is compared between the RRV-IRES-yCD2 and RRV-IRES-syCD2 groups and between the RRV-GSG-T2A-yCD2 and RRV_GSG-T2A-syCD2 groups at the 3/97 and 15/85, and 30/70 ratios, the data indicate that mice bearing tumors transduced with the syCD2 variants in both cases have a significant higher survival benefit than mice bearing the tumor transduced with the yCD2 version. This seen more clearly at the lower ratios of syCD infected cells. Our data indicate that expression of a secreted prodrug activating enzyme is advantageous. This could be due several factors including: avoidance of immediate high concentration of intracellular 5-FC leading to early depletion of virus-producing cells, thus impeding further viral spread; and/or the further diffusion of CD protein and hence further diffusion of lethal concentrations of 5-FU.

1. A recombinant replication competent retrovirus comprising:
a retroviral GAG protein;
a retroviral POL protein;
a retroviral envelope;
a retroviral polynucleotide comprising Long-Terminal Repeat (LTR) sequences at the 3′ end of the retroviral polynucleotide sequence, a promoter sequence at the 5′ end of the retroviral polynucleotide, said promoter being suitable for expression in a mammalian cell, a gag nucleic acid domain, a pol nucleic acid domain and an env nucleic acid domain;
a cassette comprising a 2A peptide or 2A peptide-like coding sequence followed by a secretory signal peptide coding sequence operably linked to a heterologous polynucleotide, wherein the cassette is positioned 5′ to the 3′ LTR and is operably linked and 3′ to the env nucleic acid domain encoding the retroviral envelope; and
cis-acting sequences necessary for reverse transcription, packaging and integration in a target cell.

2. The recombinant replication competent retrovirus of claim 1, wherein the envelope is chosen from one of amphotropic, polytropic, xenotropic, 10A1, GALV, Baboon endogenous virus, RD114, rhabdovirus, alphavirus, measles or influenza virus envelopes.

3. The retrovirus of claim 1, wherein the retroviral polynucleotide sequence is engineered from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus(REV), or Gibbon ape leukemia virus (GALV).

4. The retrovirus of claim 1, wherein the retrovirus is a gammaretrovirus.

5. The retrovirus of claim 1, wherein the target cell is a mammalian cell.

6. The retrovirus of claim 1, wherein the 2A peptide or 2A peptide like coding sequence encodes a peptide containing the sequence of SEQ ID NO:1.

7. The retrovirus of claim 1, wherein the 2A peptide or 2A peptide-like coding sequence encodes a peptide of any one of SEQ ID Nos: 55-125.

8. The retrovirus of claim 1, wherein the 2A peptide or 2A peptide-like coding sequence comprises a sequence as set forth in any one of SEQ ID Nos: 8-19.

9. The retrovirus of claim 1, wherein the heterologous polynucleotide is >500 bp.

10. The retrovirus of claim 1, wherein the heterologous polynucleotide comprises at least 2 coding sequences.

11. The retrovirus of claim 1, further comprising a second cassette comprising a 2A peptide or 2A peptide-like coding sequence downstream of the cassette.

12. The retrovirus of claim 1, wherein the secretory signal peptide coding sequence encodes a peptide comprising a sequence selected from the group consisting of SEQ ID NO:289-301 and 302.

13. The retrovirus of claim 1, wherein the heterologous polynucleotide encodes an antibody, antibody fragment, scFv, antigen binding domain or peptide cognate to a biological molecule.

14. The retrovirus of claim 1, wherein the heterologous polynucleotides comprises a sequence that encodes a secretory signal peptide operably linked to a heterologous protein or polypeptide, wherein the heterologous protein or polypeptide is selected from the group consisting of a prodrug activating enzyme, a cytokine, a receptor ligand, an immunoglobulin derived binding polypeptide, a non-immunoglobulin binding polypeptide, and any combination thereof wherein multiple heterologous proteins or polypeptide are separated by a 2A or 2A-like peptide.

15. The retrovirus of claim 1, wherein the retrovirus further comprises a second cassette comprising an internal promoter or gene expression element operably linked to a different heterologous polynucleotide downstream of the cassette.

16. (canceled)

17. The retrovirus of claim 1, wherein the gag nucleic acid domain is derived from a gammaretrovirus.

18. (canceled)

19. The retrovirus of claim 1, wherein the pol nucleic acid domain is derived from a gammaretrovirus.

20. (canceled)

21. The retrovirus of claim 1, wherein the env nucleic acid domain comprises a sequence from about nucleotide number 6359 to about nucleotide 8323 of SEQ ID NO:2, wherein T can be U or a sequence having at least 95%, 98%, 99% or 99.8% identity thereto.

22. The retrovirus of claim 1, wherein the LTR sequence at the 3′ end is derived from a gammaretrovirus.

23. (canceled)

24. The retrovirus of claim 1, wherein the heterologous nucleic acid sequence encodes a biological response modifier or an immunopotentiating cytokine.

25. (canceled)

26. The retrovirus according to claim 1, wherein the heterologous nucleic acid encodes a polypeptide that converts a nontoxic prodrug in to a toxic drug.

27. (canceled)

28. The retrovirus according to claim 1, wherein the heterologous nucleic acid sequence encodes a receptor domain, an antibody, an antibody fragment, or a non-immunoglobulin binding domain.

29. A recombinant polynucleotide for producing a retrovirus of claim 1.

30-35. (canceled)

36. The retrovirus of claim 1, wherein the retrovirus and/or the polynucleotide have been engineered to remove tryptophan codons susceptible to human APOBEC hypermutations.

37-38. (canceled)
